(19) United States
(12) Patent Application Publication
(10) Pub. No.: US 2019/0275206 A1
(43) Pub. Date: Sep. 12, 2019

(54) TISSUE IMPLANTS AND USES THEREOF

(71) Applicant: Biologica Technologies, Carlsbad, CA (US)

(72) Inventors: Amit Prakash Govil, Carlsbad, CA (US); Bryan Choi, Carlsbad, CA (US); Sahil Jalota, Carlsbad, CA (US); Hanson Lee, Carlsbad, CA (US); Justin Provencher, Carlsbad, CA (US)

(21) Appl. No.: 16/349,848

(22) PCT Filed: Nov. 15, 2017
(86) PCT No.: PCT/US2017/061830

§ 371 Date: May 14, 2019

Related U.S. Application Data
(60) Provisional application No. 62/422,463, filed on Nov. 15, 2016.

(51) Int. Cl. A61L 27/38; A61K 35/28; A61K 9/00; A61L 27/54; A61K 38/18

(52) U.S. Cl. CPC A61L 27/3834; A61K 35/28; A61K 35/35; A61L 27/54; A61K 38/18; A61K 9/0024

(57) ABSTRACT

Provided herein are tissue implants and uses thereof. In certain aspects, tissue implants are described that can be used to help repair, rejuvenate, and/or revitalize the scalp. Also provided herein are methods of making, use, and administration thereof. The tissue implants can be prepared by harvesting cells or tissue from a donor and selectively lysing the cells or tissue to obtain the intracellular content. Also provided herein are delivery devices for delivering the tissue implants described herein and kits that include the tissue implants described herein.

### CROSS-REFERENCE TO RELATED APPLICATIONS

This application claims the benefit of U.S. provisional patent application Ser. No. 62/422,463 filed on Nov. 15, 2016, having the title “TISSUE IMPLANTS AND USES THEREOF,” the disclosure of which is incorporated herein in its entirety.

### BACKGROUND

Hair loss and/or slowing of hair growth can occur as a result of the natural aging process as well as gradual phenotypic expression of adverse genetic factors in addition to traumatic events, such as surgery, disease, or other conditions. Hair loss and/or stunted hair growth can lead to undesirable effects in an individual experiencing such. For example, hair loss and/or stunted hair growth can alter the appearance of an individual, negatively affecting the inward and outward perception of said individual. Besides impacting perception, negative effects of hair loss and/or stunted hair growth can have additional consequences such as the development of mood disorders such as depression. In individuals experiencing hair loss as a result of treatment for an illness, such as radiation and chemotherapy for cancer, mood disorders developed in part from hair loss can further impair the recovery of such individuals.

In such instances, tissue implants are desirable to address some of the deleterious consequences of hair loss. Research into hair biology and hair loss is a relatively small field, and many off the shelf therapeutics for improving hair growth are unproven and unsuccessful. As such, there exists a need for improved tissue implants, as well as methods of making tissue implants in addition to methods for delivery of tissue implants.

### SUMMARY

Described herein are tissue implants and uses thereof. Described herein is a method of improving hair growth or hair quality in a subject in need thereof. Methods as described herein can comprise delivering a tissue implant to a subject in need thereof by a delivery method in an amount effective to improve hair growth or hair quality. The tissue implant can comprise cell lysate comprising a bioactive intracellular component.

Tissue implants as described herein can be derived from an autologous donor, an allogeneic donor, a xenogeneic donor, a syngeneic donor, and combinations thereof. Tissue implants as described herein can be derived from a physiological solution comprising blood cells, bone marrow, bone marrow cells, amniotic fluid, amniotic fluid cells, amnion, amnion ECM, placenta, placental ECM, muscle, muscle ECM, interstitial fluid, stromal vascular fraction, or synovial fluid, individually or in combination. Cell lysate of tissue implants as described herein can be derived from tissue containing one or more adipose cells, tissue containing one or more bone marrow cells, tissue containing one or more amnion cells, tissue containing one or more blood cells, tissue containing one or more dermal cells, or combinations thereof. The cell lysate can be derived from mesenchymal stem cells. The cell lysate can be derived from adipose derived stem cells.

Tissue implants as described herein can further comprise one or more of: a delivery enhancer, amino acid, peptide, flow enhancer, preservative, storage agent, protease inhibitor, or a stabilizer, individually or in combination.

The delivery method of tissue implants to subjects in need thereof can be surgical implantation, subdermal injection, topical application, microneedling, transdermal application, or combinations thereof.

Tissue implants can be terminally sterilized, cross-linked, or both using irradiation or chemical means. The irradiation is gamma irradiation, x-ray irradiation, uv irradiation, or ebeam irradiation.

Tissue implants as described herein can further comprise a carrier substrate. The carrier substrate can be selected from the group consisting of: a complete extracellular matrix, a decellularized extracellular matrix, extracellular matrix components, a hydrogel, an amino acid, a polymer solid, a polymer semi-solid, a carbohydrate, self-assembling peptides, carbon nanotubes, chitosan, alginate, bone powder, cartilage powder, a protein, a sugars, a plastic, a metal, a collagen, and combinations thereof.

Tissue implants as described herein can comprise a bioactive intracellular component. The bioactive intracellular component can be contained in a slurry, and wherein the slurry ratio of slurry to carrier substrate is about 100:1 (v/v) to about 1:100 (v/v). The bioactive intracellular component can be present in the tissue implant at a concentration of at least at least 1 pg/g or at least pg/mL. The bioactive intracellular component can be present in the tissue implant at a concentration of about 0.01 pg/g to about 100 mg/g or about 0.01 pg/mL to about 100 mg/mL. The bioactive intracellular component can be present in the tissue implant at a concentration of at least about 0.01 pg/mL to about 22,000,000 mg/g.

As described herein, the amount effective to improve hair or hair quality can be a concentration of the bioactive intracellular component of at least about 0.01 pg/mL to about 50,000,000 pg/mL. The amount effective to improve hair or hair quality can be a concentration of the bioactive intracellular component of at least about 0.01 pg/mL to about 50,000,000 pg/mL and can be delivered in a volume of about 0.01 cc to about 100 cc.

The bioactive intracellular component can be a platelet-derived growth factor, a hepatocyte growth factor, an insulin growth factor, an angiopoietin, a fibronectin, a transforming growth factor, a nerve growth factor, a fibronectin, an integrin, a bone morphogenetic protein, an epidermal growth factor, an insulin-like growth factor, a fibroblast growth factor, vascular endothelial growth factor, osteoprotegerin, and osteopontin, and combinations thereof. The bioactive intracellular component can be insulin like growth factor-1 which, in certain aspects, can be present at a concentration of at least 1 pg/g or 1 pg/mL. The bioactive intracellular component can be β-fibroblast growth factor which, in certain aspects, can be present at a concentration of at least 1 pg/g or 1 pg/mL. The bioactive intracellular component can be vascular endothelial growth factor which, in certain aspects, can be present at a concentration of at least 1 pg/g or 1 pg/mL. The bioactive intracellular component can be acidic fibroblast growth factor and is present at a concentration of at least 1 pg/g or 1 pg/mL. The bioactive intracellular component can be basic fibroblast growth factor and is present at a concentration of at least 1 pg/g.

Methods as described herein can further comprise adding a compound from the group consisting of: preservatives, antibiotics, antivirals, antifungals, pH stabilizers, osmostablizers, anti-inflammants, anti-neoplastics, growth factors, angiogenic compounds, vasculogenic compounds, chemotherapeutics, immunomodulators, chemoattractants, and combinations thereof to the bioactive intracellular component, the carrier substrate, or the combined bioactive intracellular component-carrier substrate.

The delivery of tissue implants to the subject in need thereof according to methods herein can be a daily delivery, a weekly delivery, a bi-weekly delivery, a monthly delivery, a quarterly delivery, a semi-annual delivery, an annual delivery, or combinations thereof.

The delivery can extend radially, tangentially, or in another direction from a focal point within a region of interest in the subject in need thereof. Multiple deliveries can be spaced at intervals, which can be regular or irregular intervals.

Tissue implants as described herein can be a cellular implant, an acellular implant, or both and can further comprise a nutrient, a vitamin, or both.

Tissue implants as described herein can further comprise buflomedyl, vitamin B1, vitamin B6, vitamin H, vitamin C, vitamin E, coenzyme G10, amino acids, antioxidants, or antibiotics, individually or in combination.

Tissue implants as described herein can be administered according to methods as described herein in an amount effective to improve hair growth or hair quality. The amount effective to improve hair growth or hair quality can be an amount effective to increase a total protein content of the hair in the skin of the subject in need thereof. The amount effective to improve hair growth or hair quality can be the amount effective to increase a follicle density in the skin of the subject in need thereof from a first density to a second density. The amount effective to improve hair growth or hair quality can be the amount effective to increase an average hair shaft diameter in the skin of the subject in need thereof from a first diameter to a second diameter. The amount effective to improve hair growth or hair quality can be the amount effective to increase cumulative hair thickness in the skin of the subject in need thereof from a first thickness to a second thickness. The amount effective to improve hair growth or hair quality can be the amount effective to improve a coloration in the hair in the skin of the subject in need thereof by increasing luminance of a color from a first level to a second level. The amount effective to improve hair growth or hair quality can be the amount effective to improve a volume in the hair in the skin of the subject in need thereof by increasing volume of hair from a first level to a second level. The amount effective to improve hair growth or hair quality is the amount effective to improve an average length in the hair in the skin of the subject in need thereof by increasing length of hair from a first length to a second length. The amount effective to improve hair growth or hair quality can be the amount effective to improve a strength of the hair in the skin of the subject in need thereof by increasing hair strength from a first level to a second level.

Also described herein are kits for increasing hair growth or improving hair quality. Kits as described herein can comprise one or more dosages of tissue implants as described herein, wherein each of the one or more dosages contains an effective amount of tissue implants as described herein. In certain aspects, kits can also comprise delivery devices according to methods of the present disclosure.

### BRIEF DESCRIPTION OF THE DRAWINGS

Further aspects of the present disclosure will be readily appreciated upon review of the detailed description of its various embodiments, described below, when taken in conjunction with the accompanying drawings.

FIG. 1 is a flow diagram illustrating embodiments of a method for harvesting soft tissue cells and retaining endogenous intracellular components.

FIG. 2 is a flow diagram illustrating embodiments of a method of incorporating the stored or un-stored slurry of FIG. 1 into a carrier substrate.

FIG. 3 is a flow diagram illustrating embodiments of a method of incorporating the stored or un-stored slurry of FIG. 1 into a soft tissue graft.

FIG. 4 shows one embodiment of a delivery device containing a slurry as produced according to the methods described herein.

FIG. 5 shows another embodiment of a delivery device containing a slurry as produced according to the methods described herein.

FIG. 6 demonstrates increased growth factor content in a carrier substrate combined with adipose-derived intracellular compounds (LipoAmp) as compared to control.

FIG. 7 shows in vivo implantation volume of a carrier substrate combined with adipose-derived intracellular compounds (LipoAmp) over time as compared to donor matched control implants.

FIGS. 8A and 8B show control staining (FIG. 8A) and hematoxylin and eosin staining demonstrating ectopic adipogenesis at the site of implantation of a carrier substrate containing adipose-derived intracellular compounds (LipoAmp).

FIG. 9 is a flow diagram showing one embodiment of a method to produce soluble soft tissue protein compositions.

FIG. 10 is a flow diagram showing another embodiment of a method to produce soluble soft tissue protein compositions.

FIG. 11 is a flow diagram showing another embodiment of a method to produce soluble soft tissue protein compositions.

FIG. 12 is a flow diagram showing another embodiment of a method to produce soluble soft tissue protein compositions.

FIG. 13 is a flow diagram showing another embodiment of a method to produce soluble soft tissue protein compositions.

FIG. 14 is a flow diagram showing another embodiment of a method to produce soluble soft tissue protein compositions.

FIG. 15 is a flow diagram showing another embodiment of a method to produce soluble soft tissue protein compositions.

FIG. 16 is a flow diagram showing another embodiment of a method to produce soluble soft tissue protein compositions.

FIG. 17 is a flow diagram showing another embodiment of a method to produce soluble soft tissue protein compositions.

FIG. 18 is a flow diagram showing another embodiment of a method to produce soluble soft tissue protein compositions.

FIG. 19 is a flow diagram showing another embodiment of a method to produce soluble soft tissue protein compositions.

FIG. 20 is a flow diagram showing one embodiment of a method to produce soluble bone marrow derived proteins.

FIG. 21 is a flow diagram showing another embodiment of a method to produce soluble bone marrow derived proteins.

FIG. 22 is a flow diagram showing another embodiment of a method to produce soluble bone marrow derived proteins.

FIG. 23 is a flow diagram showing another embodiment of a method to produce soluble bone marrow derived proteins.

FIG. 24 is a flow diagram showing another embodiment of a method to produce soluble bone marrow derived proteins.

FIG. 25 is a flow diagram showing another embodiment of a method to produce soluble bone marrow derived proteins.

FIG. 26 is a flow diagram showing another embodiment of a method to produce soluble bone marrow derived proteins.

FIG. 27 is a flow diagram showing another embodiment of a method to produce soluble bone marrow derived proteins.

FIG. 28 is a flow diagram showing another embodiment of a method to produce soluble bone marrow derived proteins.

FIG. 29 is a flow diagram showing another embodiment of a method to produce soluble bone marrow derived proteins.

FIG. 30 is a flow diagram showing another embodiment of a method to produce soluble bone marrow derived proteins.

FIG. 31 demonstrates total protein concentration obtained by a method described herein.

FIG. 32 demonstrates the concentration of BMP-2 protein in a soluble bone marrow compositions described herein derived from various bone marrow donors.

FIG. 33 demonstrates the concentration of various proteins present in a soluble bone marrow composition from various donors.

FIG. 34 demonstrates the concentration of BMP-2 ug/g of a soluble bone marrow protein composition (ProteiOS) from various donors.

FIG. 35 demonstrates the concentrations of various bioactive factors (ng/g) of a soluble bone marrow protein composition (ProteiOS).

FIG. 36 shows a graph demonstrating BMP-2 content in a soluble bone marrow protein composition per cc of starting bone material obtained under different embodiments of a process to obtain the soluble bone marrow protein composition.

FIG. 37 shows a graph comparing BMP-2 content in a soluble bone marrow protein composition per cc of starting bone material under different processing conditions that include, inter alia, a different number of washing (or rinsing) steps.

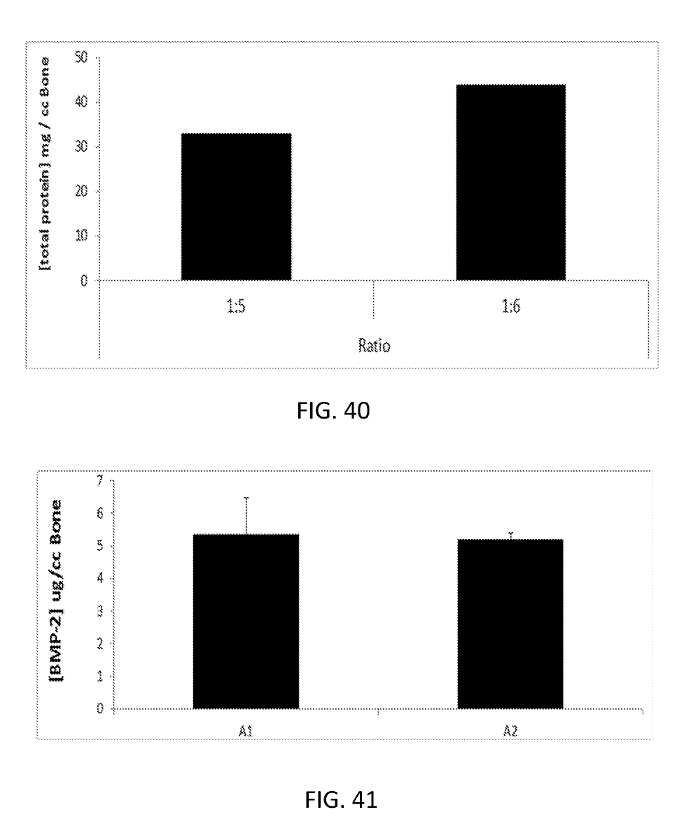

FIG. 38 shows a graph comparing total protein content in a soluble bone marrow protein composition per cc of starting bone material under different processing conditions that include, inter alia, a different number of washing (or rinsing) steps.

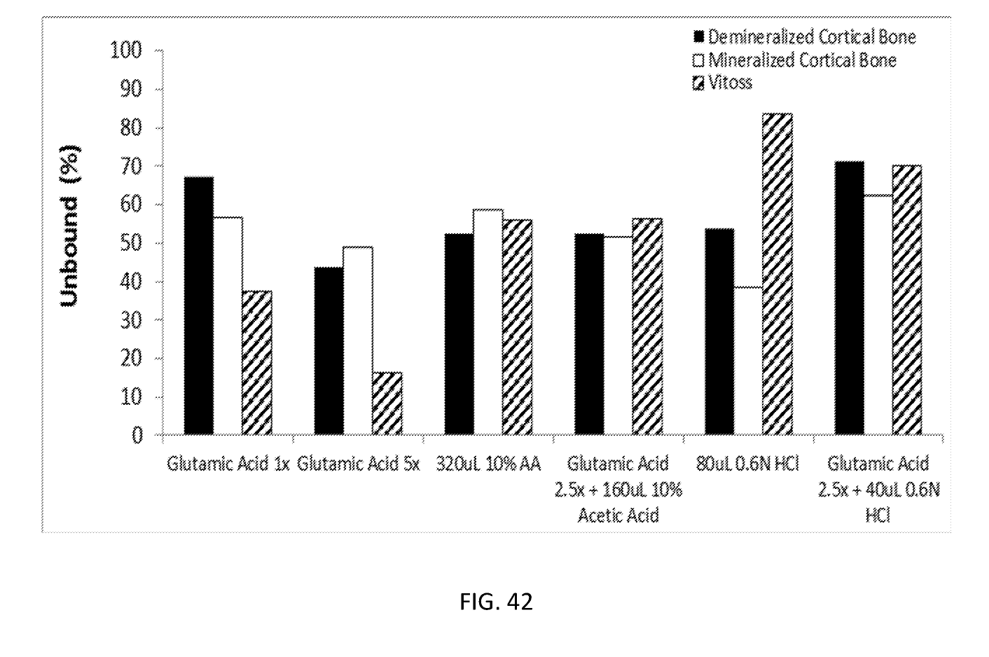

FIG. 39 shows a graph comparing BMP-2 protein content in a soluble bone marrow protein composition processed at different ratios of starting bone material to initial processing solution.

FIG. 40 shows a graph comparing total protein content in a soluble bone marrow protein composition processed at different ratios of starting bone material to initial processing solution.

FIG. 41 shows a graph demonstrating BMP-2 content in duplicate preparations of a soluble bone marrow protein composition prepared using a using a high volume of processing solution (about 1000 mL).

FIG. 42 shows a graph demonstrating the effect of a stabilizer component on binding to various graft scaffolds.

FIG. 43 is a graph showing a sampling of proteins in Example 15 identified with mass spectrometry.

FIG. 44 illustrates the relative quantification of some of the proteins listed in Example 16.

FIG. 45 is a flow diagram illustrating one embodiment in accordance with the present disclosure.

FIG. 46 is a flow diagram illustrating one embodiment in accordance with the present disclosure.

FIG. 47 is a flow diagram illustrating one embodiment in accordance with the present disclosure.

FIG. 48 is a flow diagram illustrating one embodiment in accordance with the present disclosure.

FIG. 49 is a flow diagram illustrating one embodiment in accordance with the present disclosure.

FIG. 50 is a flow diagram illustrating one embodiment in accordance with the present disclosure.

FIG. 51 is a flow diagram illustrating one embodiment in accordance with the present disclosure.

FIGS. 52-53 are flow diagrams illustrating methods to produce various embodiments of chitosan/mineral putty in accordance with the present disclosure.

FIGS. 54-56 are flow diagrams illustrating methods to produce various embodiments of chitosan/mineral scaffold sponge in accordance with the present disclosure.

FIG. 57 is a flow diagram illustrating methods to produce various embodiments of a chitosan/bone scaffold sponge containing cells in accordance with the present disclosure.

FIG. 58 is a table illustrating examples of material properties in accordance with various embodiments of the present disclosure.

FIGS. 59-60 are graphs illustrating examples of scaffold expansion in accordance with various embodiments of the present disclosure.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of molecular biology, physiology, modern surgical techniques, microbiology, nanotechnology, organic chemistry, biochemistry, botany and the like, which are within the skill of the art. Such techniques are explained fully in the literature.

### Definitions

In describing the disclosed subject matter, the following terminology will be used in accordance with the definitions set forth below.

As used herein, “about,” “approximately,” and the like, when used in connection with a numerical variable, generally refers to the value of the variable and to all values of the variable that are within the experimental error (e.g., within the 95% confidence interval for the mean) or within .+−0.10% of the indicated value, whichever is greater.

As used herein, ““effective amount” is an amount sufficient to effect beneficial or desired results. An effective amount can be administered in one or more administrations, applications, or dosages.

As used herein, “therapeutic” refers to treating or curing a disease or condition.

As used herein, “preventative” refers to hindering or stopping a disease or condition before it occurs or while the disease or condition is still in the sub-clinical phase.

As used herein, “concentrated” used in reference to an amount of a molecule, compound, or composition, including, but not limited to, a chemical compound, polynucleotide, peptide, polypeptide, protein, antibody, or fragments thereof, that indicates that the sample is distinguishable from its naturally occurring counterpart in that the concentration or number of molecules per volume is greater than that of its naturally occurring counterpart.

As used herein, “isolated” means separated from constituents, cellular and otherwise, with which the polynucleotide, peptide, polypeptide, protein, antibody, or fragments thereof, are normally associated in nature. A non-naturally occurring polynucleotide, peptide, polypeptide, protein, antibody, or fragments thereof, does not require “isolation” to distinguish it from its naturally occurring counterpart.

As used herein, “diluted” used in reference to an amount of a molecule, compound, or composition including but not limited to, a chemical compound, polynucleotide, peptide, polypeptide, protein, antibody, or fragments thereof, that indicates that the sample is distinguishable from its naturally occurring counterpart in that the concentration or number of molecules per volume is less than that of its naturally occurring counterpart.

As used interchangeably herein, “subject,” “individual,” or “patient,” refers to a vertebrate, preferably a mammal, more preferably a human. Mammals include, but are not limited to, murines, simians, humans, farm animals, sport animals, and pets. The term “pet” includes a dog, cat, guinea pig, mouse, rat, rabbit, ferret, and the like. The term farm animal includes a horse, sheep, goat, chicken, pig, cow, donkey, llama, alpaca, turkey, and the like.

As used herein, “biocompatible” or “biocompatibility” refers to the ability of a material to be used by a patient without eliciting an adverse or otherwise inappropriate host response in the patient to the material or a derivative thereof, such as a metabolite, as compared to the host response in a normal or control patient.

As used herein, “cell,” “cell line,” and “cell culture” include progeny. It is also understood that all progeny may not be precisely identical in DNA content, due to deliberate or inadvertent mutations. Variant progeny that have the same function or biological property, as screened for in the originally transformed cell, are included.

As used herein, “specific binding” refers to binding which occurs between such paired species as enzyme/substrate, receptor/agonist, antibody/antigen, and lectin/carbohydrate which may be mediated by covalent or non-covalent interactions or a combination of covalent and non-covalent interactions. When the interaction of the two species produces a non-covalently bound complex, the binding which occurs is typically electrostatic, hydrogen-bonding, or the result of lipophilic interactions. Accordingly, “specific binding” occurs between a paired species where there is interaction between the two which produces a bound complex having the characteristics of an antibody/antigen or enzyme/substrate interaction. In particular, the specific binding is characterized by the binding of one member of a pair to a particular species and to no other species within the family of compounds to which the corresponding member of the binding member belongs. Thus, for example, an antibody preferably binds to a single epitope and to no other epitope within the family of proteins.

As used herein, “control” is an alternative subject or sample used in an experiment for comparison purposes and included to minimize or distinguish the effect of variables other than an independent variable.

As used herein, “positive control” refers to a “control” that is designed to produce the desired result, provided that all reagents are functioning properly and that the experiment is properly conducted.

As used herein, “negative control” refers to a “control” that is designed to produce no effect or result, provided that all reagents are functioning properly and that the experiment is properly conducted. Other terms that are interchangeable with “negative control” include “sham,” “placebo,” and “mock.”

As used herein, “culturing” refers to maintaining cells under conditions in which they can proliferate and avoid senescence as a group of cells. “Culturing” can also include conditions in which the cells also or alternatively differentiate.

As used herein, “synergistic effect,” “synergism,” or “synergy” refers to an effect arising between two or more molecules, compounds, substances, factors, or compositions that is greater than or different from the sum of their individual effects.

As used herein, “additive effect” refers to an effect arising between two or more molecules, compounds, substances, factors, or compositions that is equal to or the same as the sum of their individual effects.

As used herein, “autologous” refers to being derived from the same subject that is the recipient.

As used herein, “allograft” refers to a graft that is derived from one member of a species and grafted in a genetically dissimilar member of the same species.

As used herein “xenograft” or “xenogeneic” refers to a substance or graft that is derived from one member of a species and grafted or used in a member of a different species.

As used herein, “autograft” refers to a graft that is derived from a subject and grafted into the same subject from which the graft was derived.

As used herein, “allogeneic” refers to involving, derived from, or being individuals of the same species that are sufficiently genetically different so as to interact with one another antigenicaly.

As used herein, “syngeneic” refers to subjects or donors that are genetically similar enough so as to be immunologically compatible to allow for transplantation, grafting, or implantation.

As used herein, “implant” or “graft,” as used interchangeably herein, refers to cells, tissues, or other compounds, including metals and plastics, that are inserted into the body of a subject.

As used herein, “filler” refers to a substance used to fill a cavity or depression. The filler can fill the depression such that it is level with the surrounding area or that the cavity is filled, such that the depth of the depression or volume of the cavity is decreased, or such that the area that was the depression is now raised relative to the areas immediately surrounding the depression.

As use herein, “immunogenic” or “immunogenicity” refers to the ability of a substance, compound, molecule, and the like (referred to as an “antigen”) to provoke an immune response in a subject.

As used herein, “exogenous” refers to a compound, substance, or molecule coming from outside a subject or donor, including their cells and tissues.

As used herein, “endogenous” refers to a compound, substance, or molecule originating from within a subject or donor, including their cells or tissues.

As used herein, “bioactive” refers to the ability or characteristic of a material, compound, molecule, or other particle that interacts with or causes an effect on any cell, tissue and/or other biological pathway in a subject.

As used herein, “bioactive factor” refers to a compound, molecule, or other particle that interacts with or causes an effect on any cell, tissue, and/or other biological pathway in a subject.

As used herein, “physiological solution” refers to a solution that is about isotonic with tissue fluids, blood, or cells.

As used herein, “donor” refers to a subject from which cells or tissues are derived.

As used herein, “slurry” refers to the resultant product from any of the methods described herein. Accordingly, the slurry can be in any form resulting from the processing described herein, including but not limited to, dehydrated slurry or tissue, paste, powder, solution, gel, putty, particulate and the like.

As used herein, “extra cellular matrix” refers to the non-cellular component surrounding cells that provides support functions to the cell including structural, biochemical, and biophysical support, including but not limited to, providing nutrients, scaffolding for structural support, and sending or responding to biological cues for cellular processes such as growth, differentiation, and homeostasis.

As used herein, “complete extracellular matrix” refers to extracellular matrix that has all components (proteins, peptides, proteoglycans, and the like) present and may or may not include other cells that are embedded in the extra cellular matrix.

As used herein, “decellularized extracellular matrix” refers to complete extracellular matrix that has been processed to remove any cells embedded within the extracellular matrix.

As used herein, “extracellular matrix component” refers to a particular component. By way of a non-limiting example, an extracellular matrix comportment can be a a specific class of comments (e.g. proteoglycans) or individual component (e.g. collagen I) that is separated or isolated from the other extracellular components. These components can be made synthetically.

As used herein “hydrogel” refers to a network of hydrophilic polymer chains that are dispersed in water. “Hydrogel” also includes a network of hydrophilic polymer chains dispersed in water that are found as a colloidal gel.

As used herein “self-assembling peptides” refer to peptides which undergo spontaneous assembly into ordered nanostructures. “Self-assembling peptides” include di-peptides, lego peptides, surfactant peptides, molecular paint or carpet peptides, and cyclic peptides.

As used herein, “adipocyte” refers to a cell type also known as a lipocyte or fat cell. Adipocytes are the cells that primarily compose adipose tissue, specialized in storing energy as fat.

As used herein, “administering” refers to an administration that is oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation or via an implanted reservoir. The term “parenteral” includes subcutaneous, intravenous, intramuscular, intra-articular, intra-synovial, intrasternal, intrathecal, intrahepatic, intralesional, and intracranial injections or infusion techniques.

As used herein, “effective amount” refers to an effective amount of tissue implants as described herein to increase follicle density, shaft diameter, and/or the rate of hair growth according to methods as described herein, or combinations thereof. An effective amount can be an amount that increases protein expression and/or protein content in the hair of a subject in need thereof. An effective amount can be an amount to increase the luminance of the hair of a subject in need thereof, the volume of the hair of a subject in need thereof, or both. An effective amount can be an amount to decrease the brittleness, improve the strength, or both of the hair of a subject in need thereof. An effective amount can be an amount to improve the cumulative density of hair on one or more desired areas of a subject in need thereof, which can be one or more regions of the scalp. An effective amount can be an amount to improve the length of one or more hairs.

### DISCUSSION

Tissue Implants and Uses Thereof

Described herein are tissue implants that can be used to help repair, rejuvenate, and/or revitalize the scalp and uses thereof. In certain aspects, tissue implants as described herein can improve hair growth. In certain aspects, tissue implants as described herein can improve hair quality (coloration, density, etc). Tissue implants as described herein can improve follicle density in the scalp of a subject. Tissue implants as described herein can provide improve vascularity and also grow/thicken hair.

Tissue implants as described herein can be made from autograft, allogeneic, or xenograft sources and may contain collagen, and growth factors/cytokines such as (but not limited to) PDGF, FGF, bFGF, aFGF, VEGF, HGF, IGF, ANG, ANG-2, fibronectin, TGFb1, etc. Components of implants as described herein can be mixed together or layered as an injectable or structured implant.

Tissue implants described herein can be implanted surgically, injected, applied topically, microneedled, and/or delivered transdermally.

Tissue implants described herein can be derived from follicular, dermis, fascia, amnion, amniotic fluid, placenta, umbilical cord, muscle, blood, bone marrow, or adipose tissue, their ECM, soluble proteins, or interacellular proteins.

In certain aspects, tissue implants as described herein can be derived from tissue that is >1% adipose; >5% adipose; >10% adipose; >20% adipose; >30% adipose; >40% adipose; >50% adipose; >60% adipose; >70% adipose; >80% adipose; or about >90% adipose.

Tissue implants as described herein can be particulated, gelatinized, solubilized, tissue pieces, or portions extracted. The implants described herein can be combined with a delivery enhancer, flow enhancer, preservative, storage agent, protease inhibitor, stabilizer, amino acids, radioprotectant, lyoprotectant, cryoprotectant, and/or the like.

Tissue implants as described herein can be derived from a physiological solution containing cells such as blood, bone marrow, interstitial fluid, stromal vascular fraction, synovial fluid, amniotic fluid, and the like.

Tissue implants as described herein can be further purified using centrifugation, fluorescence, selective lysis, chromatography, filtration, separation, and the like.

Tissue implants as described herein can be cellular (such as cellular dermis or adipose tissue) or acellular (such as acellular dermis or adipose tissue).

Tissue implants as described herein can be also contain nutrients and/or vitamins such as, but not limited to, buflomedyl, vitamin B1, B6, H, C, E, coenzyme Q10, amino acids, antioxidants, and the like.

Additionally, tissue implants as described herein can be refrigerated, frozen, or stored at ambient temperature. Tissue implants as described herein can be dehydrated via lyophilization or supplied hydrated. Tissue implants as described herein can be supplied in a syringe Or a jar/bottle/vial.

Tissue implants as described herein can be sterile filtered, tested per USP71, or terminally sterilized via irradiation (gamma, ebeam, uv, and the like). Tissue implants as described herein may be cross linked using chemical crosslinkers, heat, or irradiation (gamma, UV, ebeam, etc) to decrease degradation rate and improve volume retention.

Tissue implants as described herein can be cleaned and disinfected using detergents, peroxides, antibiotics, water, and saline.

Tissue implants as described herein can be cut into strips, sheets, or pieces. Tissue implants can be ground or blended into fine particulate. Temperature control on cutting/grinding/blending may be used to help preserve growth factor content and prevent damage or denature proteins or other components.

Tissue implant material (source tissue, final tissue implants, or anything related to thereof or in between) may be screened/seived/filtered using syringes, needles, screens, seives, or filters. Tissue implant density may be controlled by filtration, dehydration, or centrifugation speeds (100-32000 rpm/g's).

Tissue Implants as described herein may have additives such as stabilizers (radioprotectants, lyoprotectants, or cryoprotectants, such as propylene glycol, glycerol, trehlose, sucrose, amino Acids, 1-arginine, 1-lysine, polysorbate, ascorbic acid, etc. Additionally, tissue implants can be mixed prior to injection/implantation/application to improve flowability, decrease heterogenocity, and decrease particle size.

In certain embodiments, tissue implant as described herein can comprise a backbone of one or more collagens.

Also described herein are uses of tissue implants described herein. In certain aspects, uses of tissue implants as described herein relate to methods of repairing, rejuvenating, and/or revitalizing the scalp. In certain aspects, tissue implants and uses thereof are directed at the skin.

Methods as described herein can utilize tissue implants as described herein to stimulate hair growth, improve hair quality, or both in the skin and/or scalp of a subject. In certain aspects, methods as described herein can stimulate hair growth. In certain aspects, methods as described herein can improve hair quality (density, hair shaft diameter, coloration, and the like). In certain aspects, methods as described herein can stimulate one or more follicles in the skin or scalp of a subject. In certain aspects, methods as described herein can stimulate hair growth and/or improve hair quality by stimulating one or more follicles in the scalp or skin. In certain aspect, methods as described herein can stimulate angiogenesis in the skin or scalp and around follicles. In certain aspects, methods as described herein can improve angiogenesis in a subject. In certain aspects, methods as described herein may increase proliferation of cells in the scalp of a subject. In certain aspects, methods as described herein can improve angiogenesis in the scalp of an individual. In certain aspects, methods as described herein will induce no, or minimal, immune response that could adversely affect hair growth of hair quality. In certain aspects, methods as described herein can be anti-inflammatory and reduce the expression of pro-inflammatory markers in the scalp of a subject. Methods as describe herein can increase follicle density in a subject. Methods as described herein can induce cumulative thickness of the hair of an individual. Methods as described herein can increase follicle density and cumulative thickness of the hair of an individual.

Methods as described herein can deliver tissue implants as described herein to a subject in need thereof. Tissue implants can be delivered to the skin of a subject in need thereof. Tissue implants can be delivered to the scalp of an individual in need thereof. In certain aspects, without intending to be limiting, a subject in need thereof can be a male or female human. A subject in need thereof can be a subject with hair loss due to effluviums (telogen or anagen). A subject in need thereof can be a subject with alopecia (androgenic or areata). A subject in need thereof can be a subject with symptoms of hypotrichosis. A subject in need thereof can be a subject with brittle hair. A subject in need thereof can be a subject with the desire to increase follicle density, shaft diameter, and/or the rate of hair growth. A subject in need thereof can be a subject wishing to improve the quality of their hair. A subject in need thereof can be a subject wishing to improve the coloration of their hair. A subject in need thereof can be a subject wishing to decrease the brittleness, improve the strength, or both of their hair.

Tissue implants that can be delivered by methods as described herein are described in great detail below. Tissue implants employed in methods as described herein can be compositions comprising growth factors. In certain aspects, growth factor compositions may also contain cells (such as stem cells, keratinocytes, adipocytes, adipose derived stem cells, bone marrow derived stem cells, perivascular cells, stromal vascular fraction, and the like). In addition, growth factor compositions as described herein can contain ascorbic acid, hemoglobin, oxygenation molecules, vasodialators, amino acids (such as arginine, lysine, methionine, cysteine, or the remaining 16 amino acids). In certain aspects, tissue implants as described herein may contain adipose-derived stem cells and/or adipocytes. Tissue implants as described herein can be delivered to soft tissue, which in certain embodiments can be any tissue except for bone or cancellous bone. In certain embodiments, viable cells can be added to the tissue implants after the tissue implants are prepared.

Methods as described herein can administer tissue implants as described herein to the scalp of a subject in need thereof by injection, microneedling, or topical application. Tissue implants can be administered topically with or without the help of a delivery enhancer. In certain aspects, a delivery enhancer can aid in penetration of the topical application through the stratum corneum.

In an embodiment of methods as described herein, tissue implants can be injected into the scalp of a subject in need thereof with an injection device. In an embodiment, an injection device can be a syringe coupled with a hypodermic needle (of a size ranging from 0 gauge to 33 gauge on the Stubs scale).

In certain embodiments, methods as described herein can utilize a single injection of tissue implants to an area of the skin or scalp in a subject. In certain embodiments, methods as described herein can utilize multiple injections in the scalp of a subject so that tissue implants are not cleared away from the scalp by the body of the subject. In certain embodiments, injections can be spaced at intervals across a region in which a subject desires hair growth or the improvement of hair quality. In certain embodiments, methods as described herein can utilize injections at intervals of time, for example monthly, quarterly, semi-annually, or annually. The time intervals at which tissue implants are injected into a subject can be determined by a practitioner on a case-by-case basis.

The amount of tissue implants which is administered to a subject can vary and can be determined by the practitioner on an individual basis according to the subject and desired outcome. Factors which can determine the amount of tissue implants administered to a subject can include how much hair growth a subject desires, the degree to which a subject desires improvement in hair quality, and so forth. Some subjects who desire improvement in hair growth or hair quality across the whole scalp will require more tissue implants than those subjects who desire improved hair growth or improved hair quality only in a region of the scalp (due to factors such as surgical incision, for example).

As described herein, methods as described herein can deliver tissue implants to a subject in need thereof in an amount effective to increase hair growth, density, or the quality of hair. In certain aspects, without intending to be limiting, a subject in need thereof can be a male or female human according to methods as described herein. A subject in need thereof can be a subject with hair loss due to effluviums (telogen or anagen) according to methods as described herein. A subject in need thereof can be a subject with alopecia (androgenic or areata) according to methods as described herein. A subject in need thereof can be a subject with symptoms of hypotrichosis according to methods as described herein. A subject in need thereof can be a subject with brittle hair according to methods as described herein. A subject in need thereof can be a subject with the desire to increase follicle density, shaft diameter, and/or the rate of hair growth according to methods as described herein, or combinations thereof. A subject in need thereof can be a subject wishing to improve the quality of their hair. A subject in need thereof can be a subject wishing to improve the coloration of their hair. A subject in need thereof can be a subject wishing to decrease the brittleness, improve the strength, or both of their hair. A subject in need thereof can be a subject wishing to improve the cumulative density of hair on one or more desired areas.

Methods as described herein can deliver tissue implants to the skin or scalp of subjects in need thereof in an effective amount to increase follicle density, shaft diameter, and/or the rate of hair growth according to methods as described herein, or combinations thereof. An effective amount can be an amount that increases protein expression and/or protein content in the hair of a subject in need thereof. An effective amount can be an amount to increase the luminance of the hair of a subject in need thereof, the volume of the hair of a subject in need thereof, or both. An effective amount can be an amount to decrease the brittleness, improve the strength, or both of the hair of a subject in need thereof. An effective amount can be an amount to improve the cumulative density of hair on one or more desired areas of a subject in need thereof, which can be one or more regions of the scalp. An effective amount can be an amount to improve the length of one or more hairs.

As described herein, tissue implants can comprise a bioactive intracellular component. A bioactive intracellular component can be a platelet-derived growth factor, a hepatocyte growth factor, an insulin growth factor, an angiopoietin, a fibronectin, a transforming growth factor, a nerve growth factor, a fibronectin, an integrin, a bone morphogenetic protein, an epidermal growth factor, an insulin-like growth factor, a fibroblast growth factor, vascular endothelial growth factor, osteoprotegerin, and osteopontin, and various combinations thereof.

As described, an effective amount of a tissue implant can be an amount of tissue implant that contains a bioactive intracellular component at a concentration of at least at least 1 pg/g. As described, an effective amount of a tissue implant can be an amount of tissue implant that contains a bioactive intracellular component at a concentration of about 0 pg/g to about 100 mg/g. An effective amount of a tissue implant can be an amount of tissue implant comprising α-fibroblast growth factor is present at a concentration of at least 1 pg/g. An effective amount of a tissue implant can be an amount of tissue implant comprising β-fibroblast growth factor is present at a concentration of at least 1 pg/g. An effective amount of a tissue implant can be an amount of tissue implant comprising vascular endothelial growth factor is present at a concentration of at least 1 pg/g. An effective amount of a tissue implant can be an amount of tissue implant comprising acidic fibroblast growth factor and is present at a concentration of at least 1 pg/g.

An effective amount of tissue implants as described herein administered to a subject in need thereof to improve hair growth and/or hair quality can be an amount of tissue implant that contains a bioactive intracellular component at a concentration of at least at least 1 pg/mL. An effective amount of tissue implants as described herein administered to a subject in need thereof to improve hair growth and/or hair quality can be an amount of tissue implant that contains a bioactive intracellular component at a concentration of at least at least 10 pg/mL. An effective amount of tissue implants as described herein administered to a subject in need thereof to improve hair growth and/or hair quality can be an amount of tissue implant that contains a bioactive intracellular component at a concentration of at least at least 100 pg/mL. An effective amount of tissue implants as described herein administered to a subject in need thereof to improve hair growth and/or hair quality can be an amount of tissue implant that contains a bioactive intracellular component at a concentration of at least at least 1000 pg/mL. An effective amount of tissue implants as described herein administered to a subject in need thereof to improve hair growth and/or hair quality can be an amount of tissue implant that contains a bioactive intracellular component at a concentration of at least at least 10000 pg/mL. An effective amount of tissue implants as described herein administered to a subject in need thereof to improve hair growth and/or hair quality can be an amount of tissue implant that contains a bioactive intracellular component at a concentration of at least at least 100000 pg/mL.

An effective amount of tissue implants as described herein administered to a subject in need thereof to improve hair growth and/or hair quality can be an amount of tissue implant that comprises one or more of: αFGF in an amount of at least 100,000 pg/mL; βFGF in an amount of at least 100,000 pg/mL; acidic fibroblast growth factor (αFGF) in an amount of at least 100,000 pg/mL; basic fibroblast growth factor (bFGF) in an amount of at least 100,000 pg/mL; epidermal growth factor (EGF) in an amount of at least 10,000 pg/mL; hepatocyte growth factor activator (HGFa) in an amount of at least 100,000 pg/mL; hepatocyte growth factor b (HGFb) in an amount of at least 100,000 pg/mL; insulin-like growth factor 1 (IGF-1) in an amount of at least 10,000 pg/mL; platelet derived growth factor BB in an amount of at least 10,000 pg/mL; transforming growth factor β1 (TGF-β1) in an amount of at least 10,000 pg/mL; and vascular endothelial growth factor (VEGF) in an amount of at least 5,000 pg/mL. In an embodiment, an amount effective comprises VEGF in an amount of about 5,000 pg/mL to about 1,000,000 pg/mL. In an embodiment, an amount effective comprises VEGF in an amount of about 66,000 pg/mL. Effective amounts of tissue implants as described herein can be delivered to a subject in need thereof in a volume of about 0.01 cc to about 100 cc. Effective amounts of tissue implants as described herein can be delivered to a subject in need thereof in a volume of about 0.01 cc to about 1 cc. Effective amounts of tissue implants as described herein can be delivered to a subject in need thereof in a volume of about 1 cc to about 10 cc. Effective amounts of tissue implants as described herein can be delivered to a subject in need thereof in a volume of about 10 cc to about 100 cc. Effective amounts of tissue implants as described herein can be delivered to a subject in need thereof in a volume of about 10 cc. Effective amounts of tissue implants as described herein can be delivered to a subject in need thereof in a volume of about 2 cc to about 9 cc. Effective amounts of tissue implants as described herein can be delivered to a subject in need thereof in a volume of about 3 cc to about 8 cc. Effective amounts of tissue implants as described herein can be delivered to a subject in need thereof in a volume of about 4 cc to about 7 cc. Effective amounts of tissue implants as described herein can be delivered to a subject in need thereof in a volume of about 5 cc to about 6 cc. Effective amounts of tissue implants as described herein can be delivered to a subject in need thereof in a volume of about 1 cc to about 20 cc. Effective amounts of tissue implants as described herein can be delivered to a subject in need thereof in a volume of about 2 cc to about 19 cc. Effective amounts of tissue implants as described herein can be delivered to a subject in need thereof in a volume of about 5 cc to about 15 cc.

Also described herein are kits for increasing hair growth or improving hair quality. Kits as described herein can comprise one or more dosages of tissue implants as described herein, wherein each of the one or more dosages contains an effective amount of tissue implants as described herein. In certain aspects, kits can also comprise delivery devices (such as syringes and/or needles) according to methods of the present disclosure.

Tissue Implants and Methods of Preparation

As will be apparent to one of skill in the art, methods as described herein can utilize a variety of tissue implants. So that tissue implants methods according to the present disclosure can be fully realized, without intending to be limiting, embodiments of tissue implants which can be employed according to the present disclosure and their methods of preparation are described below. Tissue implants may be provided frozen, refrigerated, ambient temperature, or freeze-dried.

As described below, a variety of source tissue[s] can be utilized for tissue implants as described herein. In certain aspects soft tissue can be a source material for tissue implants as described herein. In certain embodiments, soft tissue can be adipose or bone marrow.

As described below, there are a variety of methods that can be used to prepare tissue implants according to the present disclosure as illustrated at least by the embodiments discussed below.

Soft Tissue Implants

While soft tissue implants and grafts have many applications, current methods used to harvest and prepare the soft tissues for implantation are relatively crude and harsh and, importantly, result in the loss of key proteins and other molecules. In a typical allograft harvesting and processing procedure, a donor is prepped according to standard surgical procedures and the various tissues desired are recovered by surgical staff. Recovered tissues, which are the tissue grafts, are typically cultured prior to further processing to determine the level of bacterial contamination. Some tissues can be maintained in culture to retain the tissue's viability.

Examples of these soft tissues include bone marrow, blood, adipose, skin, muscle, vasculature, cartilage, ligament, tendon, fascia, pericardium, nerve, and hair. These tissues may also include organs such as the pancreas, heart, kidney, liver, intestine, and stomach. The cells may be concentrated prior to processing as described by the current disclosure. In certain aspects, as used herein soft tissue can be any tissue containing cells may be a source of physiological fluid, such as, for example, mesodermal, endodermal, and ectodermal tissues. Examples of these tissues include bone marrow, blood, adipose, skin, muscle, vasculature, cartilage, ligament, tendon, fascia, pericardium, nerve, and hair. In certain aspects, bone, cancellous bone especially, is not a soft tissue and a tissue harvested for use with osmolarity agents intended to produce osmotic shock.

If, after culture, the soft tissue implant/graft is positive for a virulent organism, including but not limited to, Clostridia species, enterococci, or fungi, the tissue graft is discarded. However, this culture method is not completely reliable in determining bacterial contamination. Other tests on the donor, such as blood tests for HIV, hepatitis B and C, and syphilis are performed to determine the safety of the harvested allograft(s). Even these methods are not completely reliable.

As such, the allografts are typically further sterilized to reduce the microorganism contamination to less than about 10⁻³ microorganisms. Typical sterilization methods include, but are not limited to, combinations of washing with or without pressurization, centrifugation with various chemicals such as alcohols and/or detergents, and combining antibiotics with low-dose radiation. While these processing methods reduce the amount of microorganism contamination, they also can damage the tissue graft and result in the loss of many intracellular proteins and molecules.

On the one hand, the removal of intracellular proteins and molecules is good insofar as it reduces the immunogenicity of the allograft. Immunogenicity is reduced because immunogenic extracellular components (e.g. proteins, lipoproteins, and other immunogenic molecules that reside in/on the cell membrane) are washed away during the stringent washing steps, which typically include lysing of the cells. However, the washing and lysing also results in the loss of the intracellular components of the cell (e.g. proteins, DNA, RNA, peptides, and other molecules that are contained within the cell). The loss of some of these endogenous intracellular components, such as growth factor proteins, can adversely affect the performance of the allograft and its incorporation into the surrounding tissue. Allografting of intact cells or tissue grafts that are not acelluar is not successful due to the immunogenicity of the intact cells and cellularized tissues. These allografts are rarely successful and typically require that the recipient take immunosuppressants to maintain the allograft.

With these problems and limitations of current methods for preparing soft tissue implants and grafts in mind, the present disclosure provides methods of preparing soft tissue implants where the immunogenic portion of the cells are removed and at least a portion of the intracellular components are retained and processed into a soft tissue implant. The methods described herein are particularly suited for processing harvested adipose tissue and cells, as well as in vitro cultured adipose tissue and cells. Specifically, the methods described herein allow for collection of endogenous intracellular components of adipose cells and incorporate these components into soft tissue implants, grafts, and fillers for many reconstructive and surgical repair techniques.

In an embodiment, a soft tissue implant contains a bioactive intracellular component of an adipose cell and a carrier substrate, where the soft tissue implant is prepared by harvesting an adipose cell from a donor, selectively lysing the adipose sell to obtain the bioactive intracellular components and combining the bioactive intracellular component with a carrier substrate. In some embodiments, the soft tissue implant can be directly administered to a subject in need thereof.

In other embodiments, the soft tissue implant is a first soft tissue implant that is applied to a second soft tissue implant. The first soft tissue implant can be applied to a second soft tissue implant while the second soft tissue implant is outside the recipient of the second soft tissue implant (ex vivo). In other embodiments, the first soft tissue implant can be applied to the second soft tissue implant after the second soft tissue implant is already implanted in the recipient (in situ).

Accordingly, also provided are soft tissue implants, grafts, and fillers produced by the methods described herein. Also provided are devices for containing and/or delivering the soft tissue implants, grafts, and fillers produced by the methods described herein and kits containing the soft tissue implants, grafts, fillers and/or devices described herein. The methods, soft tissue implants, grafts, fillers, devices, and kits described herein offer several advantages to current soft tissue grafts at least insofar as they incorporate endogenous intracellular components, while minimizing the immunogenicity of the soft tissue implant.

Other compositions, compounds, methods, devices, systems, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

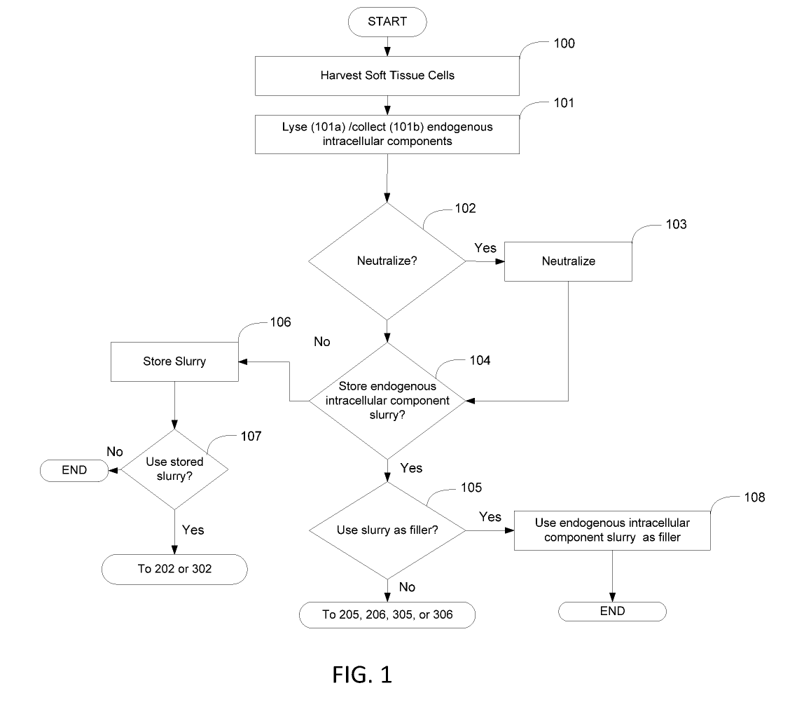

Discussion of the disclosed embodiments begins with FIG. 1, which is a flow diagram illustrating an embodiment of a method for harvesting soft tissue cells, particularly adipose cells, and collecting one or more of the endogenous intracellular components. In short, the method involves harvesting an adipose cell from a donor, selectively lysing the adipose cell to obtain a bioactive intracellular component and combining the bioactive intracellular component with a carrier substrate to form a combined bioactive intracellular component-carrier substrate. In some embodiments, the combined bioactive intracellular component-carrier substrate is administered to a subject in need thereof. The methods described herein produce a soft tissue implant containing a bioactive intracellular component of an adipose cell.

The method begins in an embodiment by harvesting cells from soft tissues from a donor or from an in vitro cell or tissue culture by a suitable method 100. Suitable harvesting methods are generally known in the art and include, but are not limited to, aspiration, scraping, dissection, and other surgical techniques known in the art. In one embodiment, tissue is excised in a desired shape and amount as determined by a medical practitioner. Factors that determine the shape and amount of the tissue to be excised include the physiological condition of the donor tissue and size of graft needed. In some embodiments, the tissue or cells are harvested at ambient temperature. In other embodiments, the tissue or cells are harvested at a temperature less than ambient temperature. In further embodiments, the tissues or cells are harvested at temperatures as low as about −210° C.

In embodiments, tissue can be minced, cut, ground, and/or chopped into particulates. In some of these embodiments, the particulates are about 1.5 times longer in one plane than another plane. In some embodiments, the elongated shape of these particulates may improve incorporation of the implant into surrounding tissue, remodeling of surrounding tissue, and tissue growth upon implantation. This may be due to an increase in surface area of the elongated implant particulates, which may facilitate vascularization.

Cutting, mincing, and grinding can further aid in separating the tissue into different constituents to further ease separation from the tissue, which allows for separation of the constituents based on density. In some embodiments, to obtain a specific constituent of tissue (e.g. adipose or collagen), the harvested tissue is cut, minced, ground, or otherwise mechanically manipulated and the constituents are separated out based on their density. In some embodiments, adipose tissue or cells are obtained from within another tissue (e.g. muscle) by this process. The profile of intracellular contents of cells can vary based on the environment in which the cell resides. Therefore, in some embodiments, the adipose cells are derived from intertissue (within or interspersed within another tissue) adipose tissue, as opposed to interstitial adipose tissue that is not interspersed within another tissue in order to obtain a particular intracellular content profile in the final implant product.

Soft tissues include, any tissue or organ that is not bone, including, but not limited to adipose tissue, muscle, cartilage, tendons, and ligaments. In one embodiment, the harvested cells are adipose cells. The soft tissues can be autologous, allogeneic, xenogeneic, or syngeneic in origin. In order to minimize immunogenicity, the use of autologous cells is most advantageous. In other words, it is preferred if the harvested cells were obtained directly or indirectly (i.e. from an in vitro culture containing cells from the subject to receive the implant) from the subject that is to receive the soft tissue implant. In an embodiment, autologous adipose cells are harvested. In other embodiments, the tissue or cells are allogeneic.

As previously mentioned, in some embodiments, the harvested soft tissue cells are cultured in vitro for an amount of time using suitable cell culture methods generally known in the art. One having ordinary skill will appreciate that the culture conditions will vary depending on the cell type. In some embodiments, cells from adipose tissue are cultured in vitro for about 1 day to about 6 months. In some embodiments, the cultured cells are harvested 100 as previously described. In an embodiment, adipose cells are harvested from a donor and cultured in vitro, until harvested 100 as previously described.

In some embodiments, the harvested cells are suspended in a physiological solution. Suitable physiological solutions include, but are not limited to, saline (about 0.9% w/v), phosphate-buffered saline, Ringer's solution, Tris-buffered saline, and HEPES (2-[4-(2-hydroxyethyl)piperazin-1-yl]ethanesulfonic acid)-buffered saline. In some embodiments, the concentration of harvested cells in the physiological solution ranges from about 1×10² cells/mL to about 1×10¹⁰ cells/mL.

Next, in some embodiments, the harvested cells are lysed 101a to release the endogenous intracellular components. After cell lysis, a cell lysate is generated, which contains the lysed cell membrane, intracellular contents, the physiological solution (if present), and the solution used to lyse the cells. The intracellular components include, but are not limited to, proteins (including enzymatic proteins and non-enzymatic proteins), protein complexes, nucleic acids, lipids, fatty acids, amino acids, peptides, simple sugars, carbohydrates, minerals, vitamins, ions (e.g. potassium, sodium, chloride, bicarbonate, magnesium, and calcium), hormones, and growth factors (which can be proteins or other types of molecules or macromolecules themselves). Examples of intracellular components include, but are not limited to αFGF, bFGF, VEGF, TGFB1, ANG, IGF, and the like. Lysing can occur by mechanical, chemical, and/or biological processes. Mechanical process include, but are not limited to, thermolysis, microfluidics, ultrasonics, electric shock, blending, milling, beadbeating, homogenization, french press, impingement, applying excessive shear, pressure, or vacuum forces, or combinations thereof.

For some embodiments, thermolysis includes freezing, freeze-thaw cycles, and heating to disrupt cell membranes. In other embodiments, microfluidics includes osmotic shock or crenation. Ultrasonic methods of lysis include, but are not limited to, sonications, sonoporation, sonochemistry, sonoluminescence, and sonic cavitation. Electric shock methods of lysis include, but are not limited to, electroporation and exposure of the cells to high voltage and amperage sources. Milling or beadbeating methods of cell lysis involve physically colliding or grinding the cells with an object or one another, in order to break the cell membranes. In some embodiments, excessive shear pressure is induced by aggressive pipetting through a small aperture centrifuging at a high rpm which results in a high gravitational force being applied to the cell, turbulent flow, or applying a vacuum to the cells, such that that the cell membranes are sheared apart.

In other embodiments, chemical methods are employed to lyse the cells. In some of these embodiments, cells are lysed after exposure to detergents, solvents, surfactants, hemolysis, or combinations thereof. Exposure to detergents and/or solvents may also disrupt cell membranes and remove lipid barriers surrounding the cells. Further, exposure to detergents, surfactants, and hemolysins can also aid in the removal of other debris that may be present in the cell solution. In other embodiments, cells are lysed due to a pH imbalance induced by exposure to an acidic (pH less than 7), basic (pH greater than 7) or neutral solution (pH equals 7). In additional embodiments, additional ions, such as sodium, potassium, and calcium, are added to the physiological solution to alter the osmolarity of the solution such that it is no longer isotonic. Examples include, but are not limited to, water, triton, peroxides, antibiotics, and other bioburden reducing solutions.

In further embodiments, the cells are lysed using a biological method or process. In some embodiments, the cells are contacted with an enzyme, such as lysozyme, mannases, proteases, lipidases, glycanases, or combinations thereof, which lyse the cell membranes. In other embodiments, viruses are employed to lyse the cell membranes.

Continuing with FIG. 1, as the endogenous intracellular components are released, at least some are collected 101b. In some embodiments, substantially all of the intracellular components are separated from the cell membrane components and collected. In other embodiments, a subset of the intracellular components is collected. In these embodiments, the desired intracellular components are collected and separated from the rest of the cell membrane fragments and/or the other intracellular components using a suitable separating technique. In these embodiments, where a selective subset of intracellular components is obtained during lysis, the steps 101a and 101b are collectively referred to as selective lysis. In some embodiments, the separated intracellular components are used in subsequent steps of the methods described herein. In other embodiments, the remaining intracellular components in the lysate are used in subsequent steps of the methods described herein. In either case, the portion containing the desired intracellular components is referred to as the endogenous intracellular component slurry in the remainder of the steps.

In some embodiments, the desired intracellular components are separated using a chromatography technique. Suitable chromatography techniques include, but are not limited to, size exclusion chromatography, ion exchange chromatography, expanded bed absorption chromatography, affinity chromatography (including but not limited to supercritical fluid chromatography), displacement chromatography, gas chromatography, liquid chromatography, column chromatography, planar chromatography (including, but not limited to paper chromatography, thin-layer chromatography), reverse-phase chromatography, simulated moving-bed chromatography, pyrolysis gas chromatography, fast protein liquid chromatography, high performance liquid chromatography, ultra-high performance liquid chromatography, countercurrent chromatography, and chiral chromatography.

In other embodiments, the desired intracellular components are separated using an immunoseparation technique. In these embodiments, antibodies specific for a particular intracellular component are employed to bind the desired intracellular component. The antibody-intracellular component complex can then be separated from the rest of the lysate using antibody purification methods known in the art. In some embodiments, the antibody-intracellular component complex is separated from the lysate by exposing the lysate to an immunoglobulin affinity column. In other embodiments, the antibody is complexes to a magnetic compound or ion. In these embodiments, the antibody-intracellular component complex is separated from the complex using a magnetic field. After separation from the lysate, the antibody can be separated from the intracellular component using techniques generally known in the art.

In other embodiments, the lysate solution is exposed to a substrate having a charged surface. Suitable substrates include, but are not limited to, ion resins, ceramics, mineralized tissues, demineralized tissues, soft tissues, metals, plastics, polymers, and combinations thereof. The surface of these substrates can inherently carry a charge or be configured such that they carry a charge. The surface of the substrate can carry a positive or negative charge. The charged surface of the substrate attracts oppositely charged intracellular components present in the lysate.

Continuing with FIG. 1, it is determined in step 102 if the lysate or separated intracellular components are to be neutralized or not. In some embodiments, the lysate or intracellular components are neutralized in step 103. In these embodiments, the pH of the lysate or a solution containing the separated desired intracellular components is adjusted to about 6 to about 8. In an embodiment, the pH of the lysate or the solution containing the separated desired intracellular components is adjusted to about 7. In one non-limiting example, HCL or acetic acid can optionally be used to render the solution more acidic or NaOH or a buffer (like PBS) may neutralize the solution or make it more basic.

In some embodiments, after neutralizing the lysate or the solution containing the separated desired intracellular components in step 103 or determining not to neutralize the lysate or the solution containing the separated desired intracellular components in step 102, it is determined in step 104 if the endogenous intracellular component slurry is to be stored or not. In embodiments where the endogenous intracellular component slurry is to be stored, the slurry is stored by a suitable method for later use in step 106. In some of these embodiments, the slurry is dehydrated (partial or complete). The dehydrated slurry can be cut to a desired shape and size. For example, the dehydrated slurry can be irregular, or about spherical, rectangular, triangular, or sheet-like. One of ordinary skill in the art will appreciate that the desired shape and size of the dehydrated slurry will depend on a variety of factors, including but not limited to, the implant use and the location of implantation. In other embodiments, the slurry is lyophilized. In some embodiments, the slurry, dehydrated slurry, or lyophilized slurry is placed in a suitable container. In some embodiments, the container is air tight. In other embodiments, the container can withstand freezing.

In some embodiments, the container contains information regarding the donor source, lot number, intracellular components contained therein, and/or other information, which identifies or otherwise characterizes the endogenous intracellular component slurry. In further embodiments, the slurry, dehydrated slurry, or lyophilized slurry is stored at about 4° C. to about −209° C. The slurry can be stored prior to use for up to about 5 years. In some embodiments, additional compounds are added to the slurry prior to storage. Suitable compounds include, but are not limited to, preservatives, cryoprotectants, diluents, antibiotics, antivirals, antifungals, pH stabilizers, osmostablizers, protease inhibitors or combinations thereof.

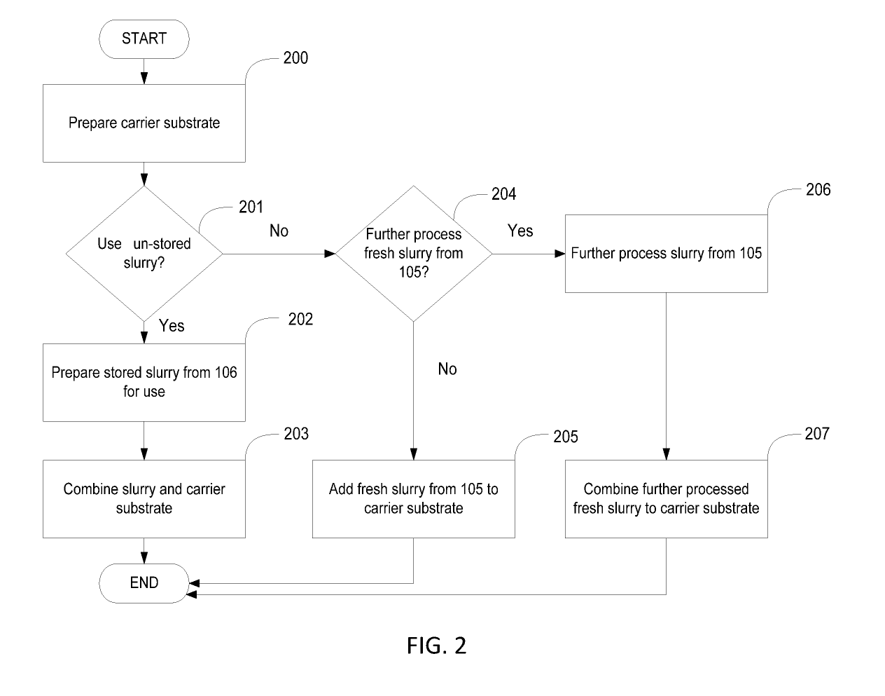

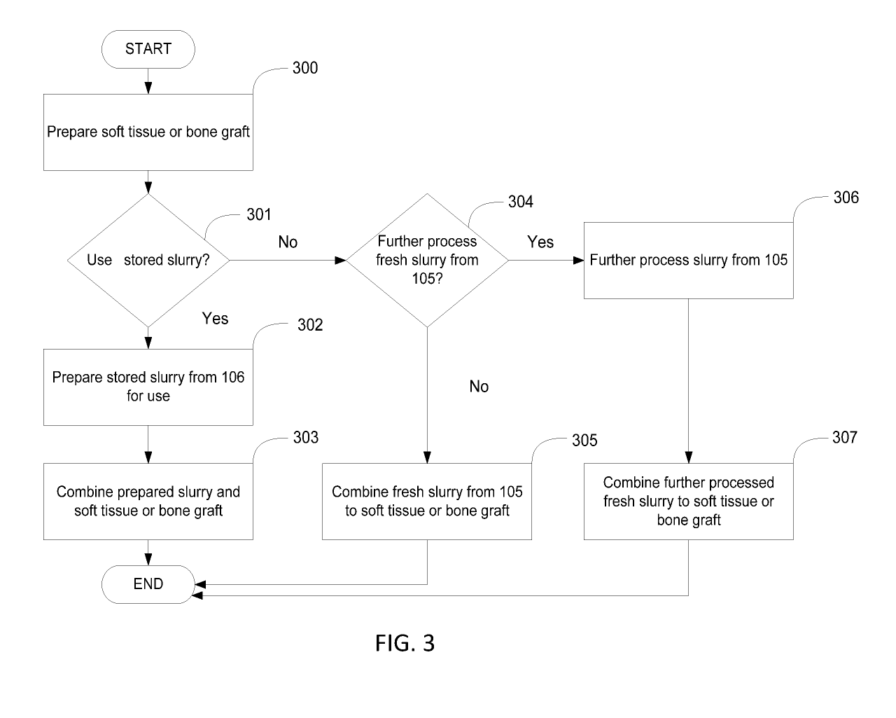

In some embodiments, it is determined in step 107 whether to use the stored slurry. In some embodiments where it is decided to use the stored slurry, the stored slurry is used in step 202 in FIG. 2. In other embodiments, the stored slurry is used in step 302 of FIG. 3.

In embodiments where it is determined in step 104 that the slurry is not to be stored, it is determined in step 105 whether to use the slurry containing endogenous intracellular components directly as filler for implantation in a subject. If it is decided to use the slurry directly as filler, the slurry is implanted into a subject as filler. In some embodiments, additional components are added to the slurry prior to use as a filler. Suitable compounds include, but are not limited to, preservatives, diluents, antibiotics, antivirals, antifungals, pH stabilizers, osmostablizers, anti-inflammants, anti-neoplastics, chemotherapeutics, immunomodulators (including immunosuppressants), chemoattractants, growth factors, anticoagulants, or combinations thereof.

In some embodiments, the slurry is implanted into a subject at a location that has been determined by a medical practitioner to be in need of a filler. In addition to providing volume to the implantation site, the filler can aid in recruitment of compounds, such as growth factors and cytokines, to the implantation site. This facilitates the growth and development of existing cells and stimulates the growth and development of new cells at the implantation site. As such, when the filler is absorbed by the body, the subject's own cells remain in place to level out the depression in the skin. In one non-limiting example, a dermatologist or reconstructive medicine practitioner determines to use the filler to add substance to depressions in skin (e.g. wrinkles) to even out the skin surface and administers the filler to a depression in the skin.

In further embodiments, the filler is administered to a location in a subject that has a tissue implant graft already in place or is added to the site of a tissue graft during the same procedure that the tissue graft is being implanted in the subject. As previously described, the filler can aid in recruitment of compounds, such as growth factors and cytokines, to the implantation site. This facilitates the growth and developments of existing cells in the area and the growth and development of new cells at the implantation site. This process also enhances integration of the tissue graft to the surrounding tissue, which improves performance of the tissue graft.

In some embodiments where it is determined not to use the slurry as filler, the slurry can be used in steps 205 or 206 of FIG. 2. In other embodiments, the slurry can be used in steps 305 or 306 of FIG. 3. In some embodiments, prior to use in steps 205, 206, 305, or 306, additional compounds are added to the slurry. Suitable compounds include, but are not limited to, preservatives, diluents, antibiotics, antivirals, antifungals, pH stabilizers, osmostablizers, anti-inflammants, anti-neoplastics, chemotherapeutics, immunomodulators (including immunosuppressants), chemoattractants, or combinations thereof.

During the generation of the slurry, the hydrophobic components of the adipose cells are separated from the hydrophilic components of the adipose cells. According to the steps previously described, the slurry contains only the hydrophilic components. However, in some embodiments, for example where increased lubricity is desired, the some of the hydrophobic components can be added back into the slurry.

Attention is now directed to FIG. 2, which is a flow diagram illustrating one embodiment of a method of incorporating the stored or un-stored slurry of FIG. 1 into a carrier substrate. As previously discussed, the slurry contains one or more intracellular components, which can enhance the performance of a soft tissue graft or implant. The embodiments discussed in relation to FIG. 2 are directed towards incorporating the intracellular components in a carrier substrate, which then can be administered to a subject in need thereof. In some embodiments, the carrier substrate is isolated along with the slurry. In other words, the slurry is generated such that it contains the carrier substrate as well as the intracellular growth factors and other hydrophilic components. In other embodiments, the slurry does not contain a carrier substrate. In either case, carrier substrate(s) can be added to the slurry as described below.

In some embodiments, the carrier substrate further enhances the performance of the soft tissue graft or implant. For example, the carrier substrate can be a scaffold, which provides an environment for cell growth and development. Suitable carrier substrates include but are not limited to, allogeneic, autologous, syngeneic, or xenogeneic complete extracellular matrix, decllularized extracellular matrix, or extracellular matrix components such as hydrogels, synthetic or natural polymer solids and semi-solids, carbohydrates, self-assembling peptides, carbon nanotubes, chitosan, alginate, hyaluronic acid, bone powder, cartilage powder, proteins, sugars, plastics, metals, or combinations thereof. In some embodiments, the carrier substrate is biocompatible. In embodiments, the carrier substrate is prepared for use 200 by methods generally known in the art. In some embodiments, the carrier substrate is already ready for use and no preparation is necessary. In some embodiments, the ratio of slurry to carrier substrate ranges from about 1:1 v/v to about 10:1 v/v. In other embodiments, the ratio of slurry to carrier substrate ranges from about 1:1 v/v to about 1:100 v/v.

After the carrier substrate is prepared 200, it is determined whether or not to use stored 106, (FIG. 1) or un-stored (fresh) 105, (FIG. 1) slurry 201. In embodiments where it is decided to use stored slurry, the stored slurry from step 106 (FIG. 1) is prepared for use in step 202. In some embodiments, preparation of the stored slurry includes thawing the slurry. In other embodiments, preparation of the stored slurry includes rehydrating the slurry. If the slurry is not rehydrated prior to use, it will become rehydrated upon introduction into the body of a subject when it contacts the biological fluids within the body. In further embodiments, the preparation process requires no additional preparation of the stored sample other than to take it from storage. After the stored slurry is prepared 202, the prepared slurry is then combined with the carrier substrate 203 using suitable methods.

In embodiments where it is decided to not to use the stored slurry, it is determined in step 204 whether to further process the fresh slurry from step 105 (FIG. 1). In embodiments where it is determined to further process fresh slurry from step 105 (FIG. 1), the slurry is further processed 206. The slurry can be further processed by filtering, concentrating, diluting, and/or fortifying with additional compounds, such as preservatives, antibiotics, antivirals, antifungals, pH stabilizers, osmostablizers, anti-inflammants, anti-neoplastics, chemotherapeutics, immunomodulators (including immunosuppressants), chemoattractants, or combinations thereof.

After further processing 206, the further processed slurry is combined with the prepared carrier substrate 207. The carrier substrate containing the slurry can then be implanted into a subject in need thereof. In some embodiments, the carrier substrate containing the slurry is implanted into a subject at a location that has been determined by a medical practitioner to be in need thereof. In addition to providing volume to the implantation site, the carrier substrate containing the slurry can aid in recruitment of compounds, such as growth factors and cytokines, to the implantation site. This facilitates the growth and development of existing cells and stimulates the growth and development of new cells at the implantation site. As such, when the carrier substrate and/or slurry is absorbed by the body, the subject's own cells remain in place to level out the depression in the skin. In one non-limiting example, a dermatologist or reconstructive medicine practitioner determines to use the carrier substrate containing the slurry to add substance to depressions in skin (e.g. wrinkles) to even out the skin surface and administers the carrier substrate containing the slurry to a depression in the skin.

In further embodiments, the carrier substrate containing the slurry or components thereof is administered to a location in a subject that has a tissue implant already in place or is added to the site of a tissue graft during the same procedure that the tissue graft is being implanted in the subject. In other embodiments, the carrier substrate containing the slurry can be added to a tissue graft prior to the tissue graft from being implanted. As previously described, the carrier substrate containing the slurry can aid in recruitment of compounds, such as growth factors and cytokines, to the implantation site. This facilitates the growth and development of existing cells in the area and the growth and development of new cells at the implantation cite. This process also enhances integration of the tissue graft to the surrounding tissue, which improves performance of the tissue graft.

In embodiments where it is determined not to further process the fresh slurry from step 105 (FIG. 1), the fresh slurry is combined with the carrier substrate 205 as previously described. The combined carrier substrate/slurry can be administered to a subject in need thereof as previously described above with respect to processed fresh slurry.

Turning now to FIG. 3, which shows a flow diagram illustrating embodiments of a method of incorporating the stored or un-stored slurry of FIG. 1 into a soft tissue graft. As previously discussed, the slurry contains one or more intracellular components, which can enhance the performance of a soft tissue graft. The method begins with preparation of a soft tissue graft 300. In some embodiments, the soft tissue graft is harvested from a donor. The soft tissue graft can be allogeneic, autologous, syngeneic, or xenogeneic. In other embodiments, the soft tissue graft is obtained from a soft tissue graft developed or maintained by in vitro or ex vivo culture. In some embodiments, the soft tissue graft is cleaned, sterilized, and/or decellularized. In some embodiments, the soft tissue graft is ready to use and no preparation steps are needed.

After the soft tissue graft is prepared 300, it is determined whether or not to use stored 106, (FIG. 1) or un-stored (fresh) 105, (FIG. 1) slurry 201. In embodiments where it is decided to use stored slurry, the stored slurry from step 106 (FIG. 1) is prepared for use in step 302. In some embodiments, preparation of the stored slurry includes thawing the slurry. In other embodiments, preparation of the stored slurry includes rehydrating the slurry. If the slurry is not rehydrated prior to use, it will become rehydrated upon introduction into the body of a subject when it contacts the biological fluids within the body. In further embodiments, the preparation process requires no additional preparation of the stored sample other than to take it from storage.

After the stored slurry is prepared 302, the prepared slurry is combined with the soft tissue graft 303 using suitable methods. In some embodiments, the slurry is combined with the soft tissue graft prior to grafting the soft tissue graft in a subject. In other embodiments, the slurry is combined with the soft tissue graft after the soft tissue graft is already in place within a subject.

In embodiments where it is decided not to use stored slurry, it is determined whether or not to further process the fresh slurry from step 105 (FIG. 1). In embodiments where it is determined to further process fresh slurry from step 105 (FIG. 1), the slurry is further processed in step 306. The slurry can be further processed by filtering, concentrating, diluting, and/or fortifying with additional compounds, such as preservatives, antibiotics, antivirals, antifungals, pH stabilizers, osmostablizers, anti-inflammants, anti-neoplastics, chemotherapeutics, immunomodulators (including immunosuppressants), angiogenic compounds, vasculogenic chemoattractants, or combinations thereof.

After further processing in step 306, the further processed slurry is combined with the prepared soft tissue graft in step 307. In some embodiments, the slurry is combined with the soft tissue graft prior to grafting the soft tissue graft in a subject. In other embodiments, the slurry is combined with the soft tissue graft after the soft tissue graft is already in place within a subject.

In embodiments where it is determined not to further process the fresh slurry from step 105, (FIG. 1), the fresh slurry is combined with the soft tissue graft 305. In some embodiments, the slurry is combined with the soft tissue graft prior to grafting the soft tissue graft in a subject. In other embodiments, the slurry is combined with the soft tissue graft after the soft tissue graft is already in place within a subject.

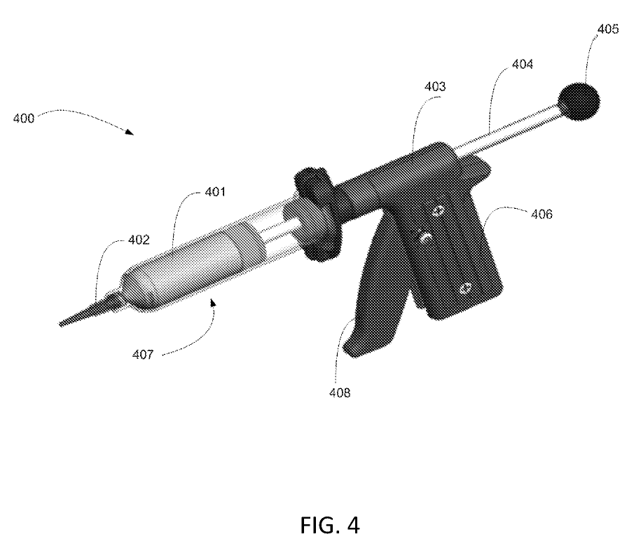

With embodiments of the methods of producing the slurry containing intracellular components, soft tissue implants and grafts combined with the slurry containing intracellular components understood, attention is directed to FIG. 4, which shows one embodiment of a delivery device 400 containing a slurry or combined slurry and carrier substrate 401, as produced according to the embodiments described herein. The delivery device 400 contains a tip 402 that is mechanically coupled to a hollow container 407. In some embodiments the tip 402 is tapered. The opening of the tip 402 can range from about 7 gauge to about 34 gauge. In some embodiments, the opening of the tip 402 is beveled. In other embodiments, the opening of the tip 402 is flush. In some embodiments, the tip 402 configured to mechanically lock onto the hollow container 407.

The hollow container 407 is configured to hold the slurry or the combined slurry and carrier substrate 401. In some embodiments, the hollow container 407 is configured to hold about 0.1 cc to about 1000 cc of slurry or the slurry combined with a carrier substrate. In one embodiment, the hollow container 407 is configured to hold up to about 1 cc of slurry or slurry/carrier substrate mixture. In another embodiment, the hollow container 407 is configured to hold up to about 5 cc of slurry or slurry/carrier substrate mixture. In yet further embodiments, the hollow container 407 is configured to hold up to about 10 cc of slurry or slurry/carrier substrate mixture. In yet further embodiments, the hollow container 407 is configured to hold up to about 20 cc of slurry or slurry/carrier substrate mixture. In other embodiments, the hollow container 407 is configured to hold up to about 50 cc of slurry or slurry/carrier substrate mixture. In still other embodiments, the hollow container 407 is configured to hold up to about 100 cc of slurry or slurry/carrier substrate mixture. In further embodiments, the hollow container 407 is configured to hold up to about 500 cc of slurry or slurry/carrier substrate mixture. In other embodiments, the hollow container 407 is configured to hold up to about 1000 cc of slurry or slurry/carrier substrate mixture.

In an embodiment, the hollow container is coupled to a handle 403 that is made up of a first grip 406 and a trigger portion 402. A movable plunger 404 is mechanically coupled to the handle 403 and hollow container 407. The movable plunger 404 extends through the handle 403 and into the end of the hollow container 407 opposite of the tip 402. The moveable plunger 404 is configured to apply positive or negative pressure to the hollow container and the contents contained therein. At the end opposite the hollow container, the movable plunger contains a second grip 405.

In some embodiments, positive pressure is applied to the hollow container by applying pressure on the second grip 405 and pushing the second grip 405 towards the handle 403. In other embodiments, the trigger 408 is squeezed. The trigger 408 is configured such that it applies a positive pressure on the plunger when the trigger 408 is squeezed. When pressure is applied to the second grip 405 or trigger 408, and the plunger end inside the hollow container 407 moves closer to the tip 402, this expels the slurry or combined slurry and carrier substrate 401 from the device 400. Negative pressure is applied by pulling on the second grip 405 and pulling the second grip 405 away from the handle 403. This moves the end of the movable plunger 404 that is inside the hollow container 407 closer to the handle 403 and away from the tip 402. Negative pressure pulls content into the hollow container 407. In further embodiments, the delivery device 400 is configured such that positive or negative pressure is generated by a machine as opposed to a human user.

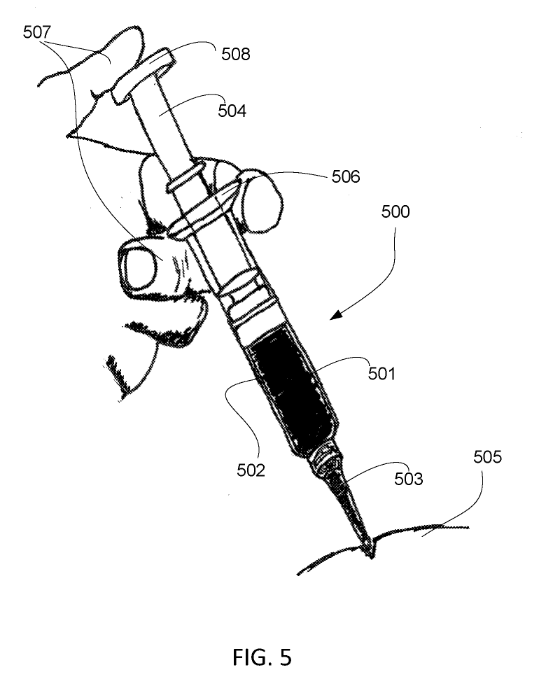

FIG. 5 shows another embodiment of a delivery device 500 containing a slurry or combined slurry and carrier substrate 501 as produced according to the methods described herein. The delivery device 500 contains a tip 503 that is mechanically coupled to a hollow container 502. In some embodiments, the tip 503 is tapered. The opening of the tip 503 can range from about 7 gauge to about 34 gauge. In some embodiments, the opening of the tip 503 is beveled. In other embodiments, the opening of the tip 503 is flush. In some embodiments, the tip 503 configured to mechanically lock onto the hollow container 503. For example, the mechanical lock can be a luer lock.

The hollow container 502 is configured to hold the slurry or the combined slurry and carrier substrate 501. In some embodiments, the hollow container 502 is coupled to a ridge portion 506 that forms a grip for fingers of a user 507 as shown in FIG. 5. A movable plunger 504 is mechanically coupled to the hollow container 502. The movable plunger 504 extends through one end of the hollow container 502 opposite of the tip 503. The moveable plunger 504 is configured to apply positive or negative pressure to the hollow container 502 and the contents contained therein. At the end opposite to the hollow container 502, the movable plunger 504 contains a thumb rest 508.

In one embodiment, positive pressure is applied to the hollow container 502 by pressure to the thumb rest 508, and thus, depresses the plunger 504 further into the hollow container 502. In some embodiments, a user holds the device 500 between two or more fingers 507. One finger 507, for example the thumb, can be placed on the thumb rest 508, while one or more other fingers 507 can be placed on either side of the hollow container 502 under the ridge portion 506, as demonstrated in FIG. 5. Positive pressure can be applied to the hollow container 502 by moving the thumb 507 closer to the other finger(s) 507 under the ridge portion 506. This depresses the plunger 504 and creates positive pressure on the hollow container 502. Negative pressure can be applied by pulling back on the plunger 504. Positive pressure expels contents 501 of the hollow container 502 and negative pressure draws contents into the hollow container 502. In some embodiments, the application of positive pressure expels the contents 501 of the hollow container 502 into a subject in need thereof 505. In further embodiments, the delivery device 500 is configured such that positive or negative pressure is generated by a machine as opposed to a human user. For example, in some embodiments the delivery device 500 is loaded into a machine, which contains portion, which applies positive pressure to the movable plunger 504. Examples of such machines are known in the art.

Also provided herein are soft tissue implants that contain a bioactive intracellular component of an adipose cell. In some embodiments, the soft tissue implant is a slurry. In one embodiment, the slurry is derived from adipocytes that are harvested from in vitro cultured adipocytes or from adipocytes harvested directly from tissue. In other embodiments, the slurry is derived from other types of soft tissue cells. Such cells include, but are not limited to, muscle, epithelial cells, tendons, and ligaments. The intracellular components contained in the slurry include but are not limited to proteins (both structural and non-structural), nucleic acids, lipids, carbohydrates, and other molecules. In some embodiments, the slurry contains an enriched or concentrated amount of these endogenous intracellular components. In some embodiments, the donor cells are selectively lysed, as previously described, such that the slurry selectively contains growth factors, particularly vascular endothelial growth factor (VEGF), basic fibroblast growth factor (bFGF), transforming growth factor beta 1 (TGFb1), acidic fibroblast growth factor (αFGF), insulin-like growth factor (IGF).

As previously discussed, an effective amount of the slurry prepared according to the methods described herein, can be administered to subjects in need thereof as a filler. In some embodiments, the slurry is configured as a paste. In other embodiments, an effective amount of the slurry can already contain and/or be combined with a carrier substrate as previously described, and the combination can then be administered to a subject in need thereof. In further embodiments, an effective amount of the slurry can be administered after placement of a soft tissue graft (other than one already incorporating the slurry). In other embodiments, an effective amount of the slurry can be incorporated directly to a soft tissue graft (that is not the slurry or slurry/carrier substrate itself) ex vivo prior to implantation. The effective dose may be between about 1 mL to 1000 ml.

The slurries (including those containing a carrier substrate), implants, and grafts and delivery devices described herein can be presented as a combination kit. As used herein, the terms “combination kit” or “kit of parts” refers to the slurries, implants, and grafts and delivery devices and additional components that are used to package, sell, market, deliver, and/or administer the combination of elements or a single element, such as the active ingredient, contained therein. Such additional components include but are not limited to, packaging, syringes, blister packages, bottles, and the like. In one embodiment the kit contains a soft tissue implant containing a bioactive intracellular component of an adipose cell, and a carrier substrate. In some embodiments, the soft tissue implant contained in the kit is generated by a method involving harvesting an adipose cell from a donor, selectively lysing the adipose cell to obtain a bioactive intracellular component, and combining the bioactive intracellular component with a carrier substrate.

In some embodiments, the combination kit also includes instructions printed on or otherwise contained in a tangible medium of expression. The instructions can provide information regarding the content of the compound or pharmaceutical formulations contained therein, safety information regarding the content of the slurry(ies), implant(s), graft(s), and delivery device(s) contained therein, information regarding the dosages, indications for use, and/or recommended treatment regimen(s) for the slurry(ies), implant(s), graft(s), and delivery device(s) contained therein. In an embodiment, the instructions provide directions for administering the soft tissue implant to a subject in need thereof as a filler or as part of a tissue graft being implanted in the subject. In some embodiments, the instructions provide directions for administering the slurry(ies), implant(s), and graft(s) to a subject in need thereof. Indications for use include, but are not limited to, reduction of fibrous capsule formation after other soft tissue implants (e.g. soft tissue (i.e., breast), vascular (i.e. stents), or joint implants) caused by the introduction of allogeneic cells or other foreign bodies, reduction of implant induced inflammation, improving implant integration into surrounding tissue, improving quality or coloring of skin, or repair of depressions in skin or other soft tissue.

Soft Tissue Protein Compositions and Methods of Making

Soft tissue grafting and implants play a role in cosmetic, reconstructive, and dental procedures. Many compositions and materials have been developed for use in soft tissue grafting and implants. Such materials include, but are not limited to, autograft, allograft, and synthetic bone graft materials. While these materials have enjoyed a certain amount of clinical success, donor morbidity when using autograft materials, adverse recipient immune response when using allograft materials, and adverse effects (e.g. scarring or other undesirable results) when using synthetic materials.

With the aforementioned shortcomings in mind, described herein are soluble soft-tissue protein compositions. The soluble soft-tissue protein compositions provided herein can, in some embodiments, overcome one or more of the shortcomings of existing soluble soft-tissue protein compositions. Also provided herein are methods of making the soluble soft tissue protein compositions. Other compositions, compounds, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

Methods of Making the Soluble Soft Tissue Protein Compositions

Described herein are methods for producing compositions containing non-recombinant (NR) soluble soft tissue proteins and/or other bioactive factor(s). The methods described herein can also result in a composition containing a dehydrated NR soluble soft tissue protein(s) and/or other bioactive factor(s). In some embodiments, the dehydrated NR soluble soft tissue protein(s) and/or other bioactive factor(s) can bind to a scaffold upon reconstitution, such as when the dehydrated soluble soft tissue protein composition comes in contact with a bodily fluid. The soluble soft tissue protein compositions prepared by the methods described herein can have a greater amount and/or concentration of soft tissue protein(s) and/or additional bioactive factor(s), and/or less immunogenicity than other osteoinductive/osteostimulatory compositions, implants, or devices incorporating complete soft tissue and/or other complete bodily fluids or tissues. The soluble soft tissue protein compositions can contain bioactive proteins.

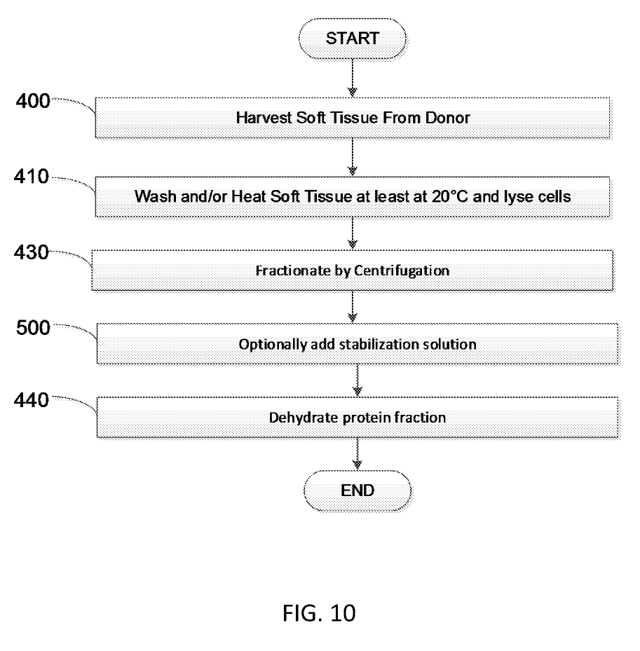

Attention is first directed to FIG. 9, which shows an embodiment of a method of producing a soluble protein composition from soft tissue. The method can begin by harvesting soft tissue from a donor 400. The donor can be a cadaver or a living subject. The donor can be a cadaver or can be a living subject. The soft tissue can be autologous, allogeneic or xenogenic. The soft tissue can be harvested in any way generally known in the art. After the soft tissue has been harvested, the soft tissue can be washed 410 in a solution. The wash solution may contain water, saline, antibiotic, antiseptic, antifungal, or crystalloid solution. In some embodiments, the wash solution is only water. Washing can take place at least at 20° C. In some embodiments, washing takes place at about 20° C. to about 37° C. In further embodiments, washing takes place at about 20° C. to about 40° C. Heating the soft tissue during washing facilitates the separation adipocytes from other types of soft tissue cells. The washing/heating step can be performed under physical agitation in a shaker incubator. In some embodiments, shaking can be conducted at about 10-300 rpm for up to about 24 hours.

During washing/heating 410, the soft tissue derived cells can be lysed. In some embodiments, the soft tissue derived cells can be lysed using a lysing solution containing an acid. In some embodiments the lysing solution can be just water. In some embodiments, the washing solution and the lysing solution can be the same solution. The acid can be acetic acid, formic acid, trichloroacetic acid, hydrofluoric acid, hydrocyanic acid, hydrogen sulfide, or hydrochloric acid. In some embodiments, the lysis solution contains about 0.001M to about 1M acetic acid. In some embodiments the lysing solution that contains the soft tissue is mixed with pre-heated water. In some embodiments, the soft tissue can be lysed for about 60 minutes. In other embodiments, the soft tissue is incubated in the lysing solution with shaking. In other embodiments, the lysing conditions can include, but are not limited to, ultrasonic techniques, thermolysis (e.g. freeze/thaw cycling), microfluidic techniques, osmotic shock, electric shock, homogenization, French press, impingement, excessive shear (e.g. aggressive pipetting through a small aperture, centrifuging at excessive revolutions per minute resulting in high gravity forces), pressure, vacuum forces, milling or bead beating techniques that physically collide or grind cells to mechanically break cell membranes, pH shock, exposure to detergents, enzymes, viruses, solvents, surfactants, hemolysins, or combinations thereof.

After washing/lysing 410, the lysate can be optionally fractionated via centrifugation 430 to separate out particles present in the lysate based on their size and/or density. Such centrifugation techniques that can be employed include, but are not limited to, differential centrifugation, rate-zonal centrifugation, and isopycnic centrifugation. In embodiments where centrifugation is used to separate particles in the lysate based on density, a suitable density gradient medium can be used. Suitable density gradient mediums include, but are not limited to, sucrose, glycerol, sorbitol, Ficoll® medium, polysucrose, dextrans, CsCl, Cs₂SO₄, KBr, Diatrizoate, Nycodenz® medium, Histodenz™ medium, iodixanol, Histopaque® mediums, ACCUSPIN® medium, and Percoll® medium. One of ordinary skill in the art will appreciate that the type of medium used is dependent on the type of particle(s) that is desired to be separated out. One or more rounds of centrifugation can be applied to the lysate to further separate out different particles in the lysate. In some embodiments, the desired fraction contains a bioactive factor, such as, but not limited to, a cytokine. In some embodiments, the lysate is centrifuge at about 100 to about 20000 rpm for about 1 to about 600 minutes. In some embodiments, the lysate is centrifuged at about 4000×g for about 10 minutes at about 4° C.

After optional fractionation 430, the desired fraction can be removed from the centrifuged lysate. In some embodiments, the desired fraction contains one or more bioactive factor, such as, but not limited to, a cytokine. The protein/bioactive factor containing fraction can then be dehydrated 440 using a suitable technique. Suitable dehydrating techniques include, but are not limited to, evaporation, vacuum drying, lyophilization, freeze drying, sublimation, and precipitation. The protein/bioactive factor containing fraction can be 0% to 100% dehydrated. After dehydration, the soluble soft tissue protein composition can contain an acid that can be diluted and/or reconstituted along with the proteins and other bioactive factors that can be present in the soluble soft tissue protein composition. In some embodiments, the protein/bioactive factor containing fraction is not dehydrated, but is kept in as a liquid and refrigerated or frozen. In some embodiments, the protein/bioactive factor containing fraction can be flash frozen in liquid nitrogen or slow frozen by placing at a temperature below 0° C., such as −10, −20, −50 or −80° C.

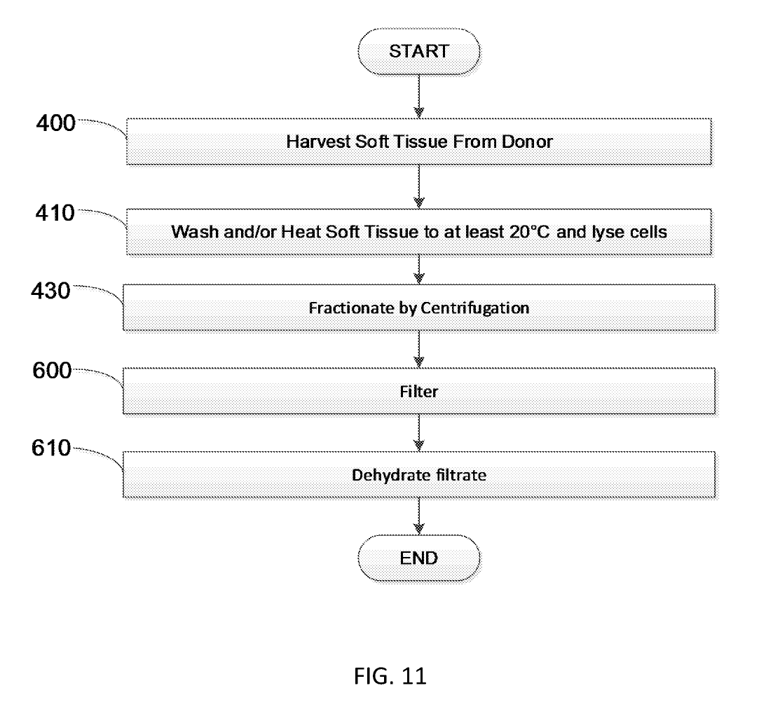

With the general process described, attention is directed to FIGS. 10-18, which demonstrate various embodiments of the general method of producing a soluble soft tissue derived soluble protein composition. Discussion begins with FIG. 10, which demonstrates embodiments of a method of generating a soluble soft tissue derived protein composition. As in FIG. 9, soft tissue can be harvested 400 and washed/heated 410 and soft tissue derived cells can be lysed. The desired components (e.g. bioactive factors) of the resulting lysate can be separated from the undesirable components using by fractionating using a suitable centrifugation technique 430. Once the desired fraction containing the proteins and/or bioactive factors of interest is obtained, the desired fraction can be dehydrated 440 using a suitable dehydration technique. As shown in FIG. 10, an optional suitable stabilization solution can be added 500 the dehydrated soft tissue derived soluble protein composition prior to dehydration 440. Suitable stabilization solutions can aid in maintaining protein integrity and activity. In some embodiments, the stabilizer can include sucrose, trehalose, glycine, L-glutamic acid, sodium chloride, polysorbate-80 and combinations thereof. The stabilization solution can contain preservatives, antibiotics, antivirals, antifungals, pH stabilizers, osmostablizers, anti-inflammants, anti-neoplastics, chemotherapeutics, immunomodulators, chemoattractants, growth factors, anticoagulants, or combinations thereof. In some embodiments, the stabilization solution per cc of final product can be about 1 mg Sucrose, 5 mg Glycine, 3.7 mg I-Glutamic Acid, 0.02 mg NaCl and 0.02 mg Polysorbate-80.

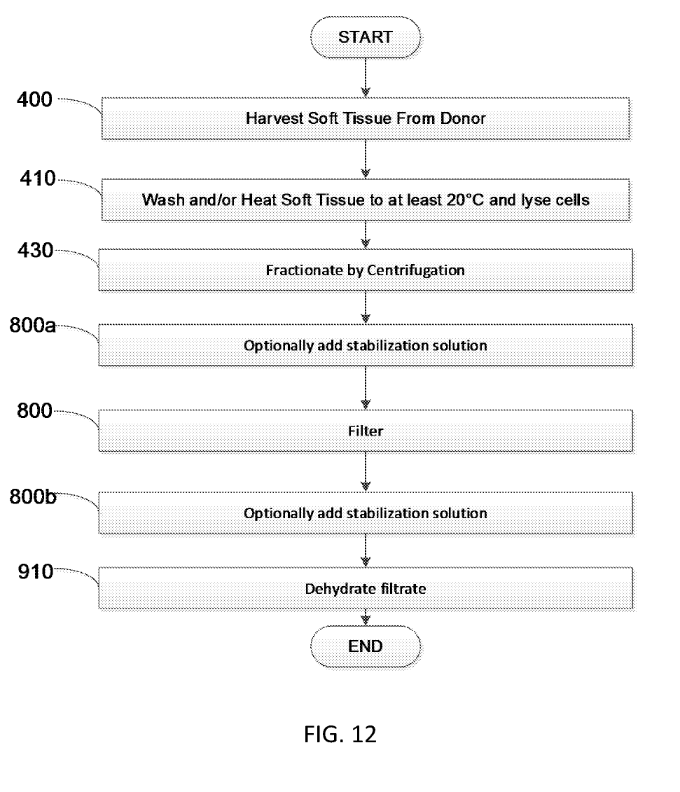

Discussion continues with FIG. 11, which shows another embodiment of a method of producing a soluble soft tissue derived soluble protein composition. As in FIG. 9, soft tissue can be harvested 400 and washed and heated and soft tissue cells can be lysed 410. The desired components (e.g. proteins and bioactive factors) of the resulting lysate can be separated from the undesirable components using by fractionating using a suitable centrifugation technique 430. As shown in FIG. 11, after fractionation by centrifugation 430 the fraction containing the desired components can be further filtered using a suitable filtration technique to remove additional undesired components that can remain in the fraction. Suitable filtration techniques can include, but are not limited to, size exclusion techniques and/or affinity purification techniques, immunoseparation techniques, and charged based separation techniques. In some embodiments, additional undesired components can include, but are not limited to, nucleic acids such as DNA and RNA, and other compounds such as hemoglobin, globin proteins, cell fragments, cell membrane molecules and other molecules that can stimulate an immune response in a subject. In some embodiments, the filter can be low protein binding. In some embodiments, the filter can be high DNA binding. In some embodiments, the filter can be high DNA binding.

Suitable materials for some filters used in the filtration step 600, include, but are not limited to, Teflon® membranes, nylon membranes, PVDF (polyvinylidene) membranes, polypropylene, cellulose acetate, PES (polyethersulfone), regenerated cellulose, glass fiber, and PTFE (polytetrafluorethylene. In some embodiments, the filter can have a size cutoff of about 0.1 to about 3.0 μM. In some embodiments multiple filters can be used, such as in a serial filtration system. In such a system, multiple types of filters can be used. The system can include at least two filters that differ in material and size cut offs. In some embodiments, polypropylene filters (e.g. size cut offs of 30 μm and 10 μm can be used), a glass fiber filter with a size cutoff of about 2.7 μm can be used, and/or a series of cellulose acetate filters (8 μm, 5 μm, 3 μm, 1.2 μm, 0.8 μm, 0.45 μm and final one of 0.2 μm) can be used to filter. The filters can be configured as syringe filters, disc filters, vacuum filter systems, bottle top vacuum filters, tube top vacuum filters, or centrifuge tube filters.

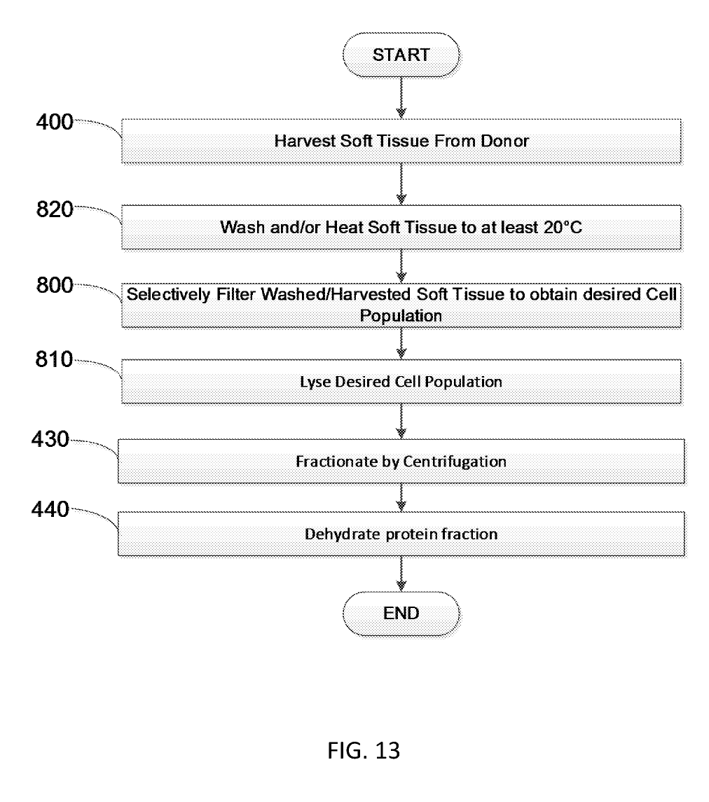

The filtrate obtained after filtering 600 can contain the desired soluble soft tissue proteins and/or other bioactive factors. The filtrate can also contain an acid. In some embodiments, the acid can be the acid that was used during the lysing step 410. The filtrate can be dehydrated 610 using any suitable dehydration techniques. Suitable dehydration techniques are described with respect to dehydrating the protein fraction 440 in FIG. 9. The filtrate can be 0% to 100% dehydrated during the dehydration step. As shown in FIG. 12, an optional suitable stabilization solution can be added 500 a,b to the product prior to dehydration 610. The stabilization solution can be added after optional centrifugation 430 and/or after filtration 600. Suitable stabilization solutions are described elsewhere herein with respect to FIG. 10.

While the soft tissue can be heated 410 to facilitate better penetration of lysing solution and/or viscosity reduction and/or separation of adipocytes from other cells that can be present the soft tissue starting material, in some instances it can be desirable to filter the harvested soft tissue prior to lysing the soft tissue desired cells to further separate adipocytes or other cell types. In some embodiments the desired cell type can be adipocytes.

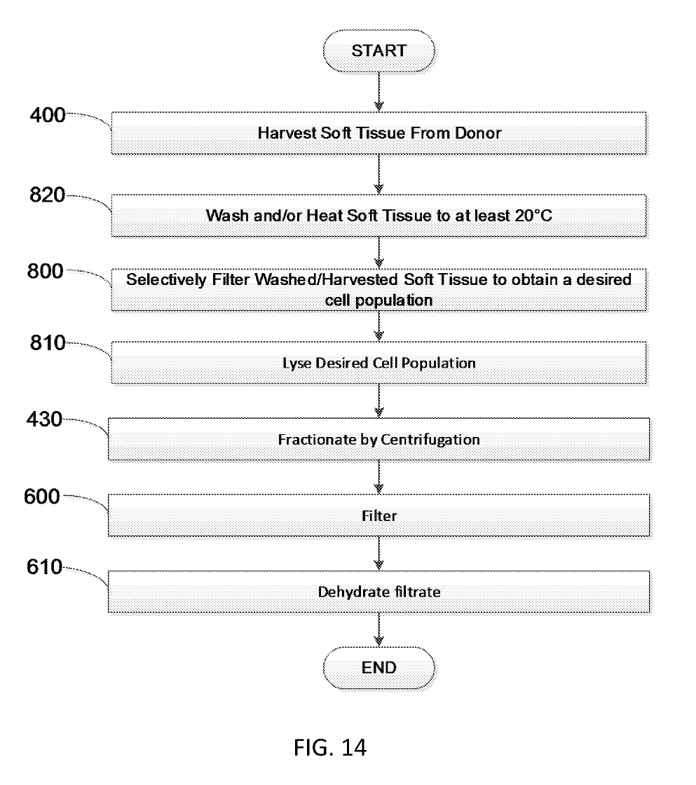

As shown in FIG. 13, soft tissue can be harvested 400 from a donor as previously described in reference to FIG. 9. The harvested soft tissue can then be washed/heated 820 as previously described with respect to FIG. 9. The washed/heated soft tissue can then be selectively filtered to obtain a desired cell population 800. The resulting desired cell population can be enriched for the desired cell type(s). In some embodiments, the resulting cell population is at least 50% to 100% of the desired cell type(s). Selective filtering can be completed by any suitable filtering techniques including, but not limited to, size exclusion separation techniques, affinity separation techniques, immunoseparation techniques, charge separation techniques, and chromatography techniques. For example, selective filtering can be achieved using osmotic lysis, cytolysis, centrifugation, size exclusion chromatography, ion exchange chromatography, expanded bed absorption chromatography, affinity chromatography (including but not limited to supercritical fluid chromatography), displacement chromatography, gas chromatography, liquid chromatography, column chromatography, planar chromatography (including, but not limited to paper chromatography, thin-layer chromatography), reverse-phase chromatography, simulated moving-bed chromatography, pyrolysis gas chromatography, fast protein liquid chromatography, high performance liquid chromatography, ultra-high performance liquid chromatography, countercurrent chromatography, chiral chromatography, and solid phase extraction. In some embodiments, where adipocytes are desired, the heating during the washing/heating step 520 is sufficient to be able to obtain an enriched population of adipocytes.

After selective filtering of the soft tissue derived cells 800, the remaining desired cell population is lysed 810. Suitable lysing techniques are described with respect to FIG. 9. After lysing, the desired cell population can be optionally fractionated 430 by centrifugation as previously described with respect to FIG. 9. Finally the obtained desired fraction containing the desired bone-marrow derived proteins and/or other bioactive factors can be dehydrated as previously described with respect to FIG. 9.

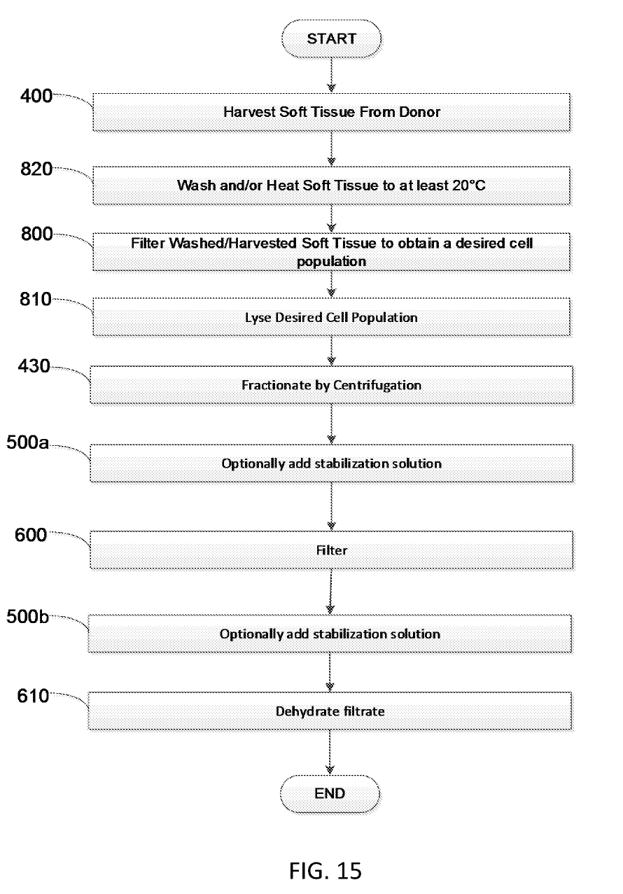

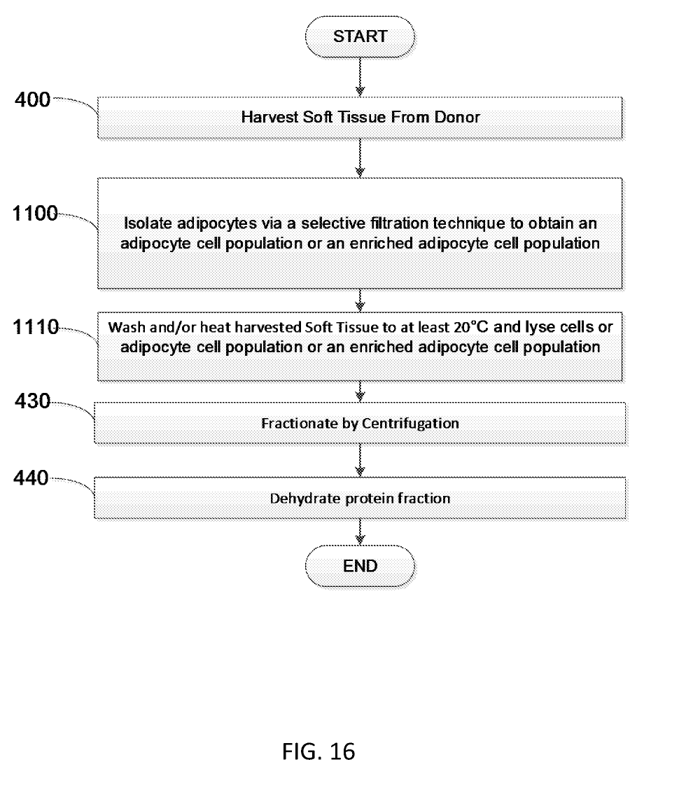

As shown in FIG. 14, the method where the harvested soft tissue can be selectively filtered 800 prior to lysing (FIG. 13) can optionally include the step of filtering 600 the obtained fraction after optional centrifugation 430. Filtering 600 can be performed as previously described with respect to FIG. 11. After filtering 600, the desired filtrate can be dehydrated 610 as previously described. As shown in FIG. 15, the methods (FIG. 13 and FIG. 14) where the harvested soft tissue can be selectively filtered 800 prior to or during lysing can also include the optional step of adding a stabilization solution 500 a,b after optional centrifugation 430 and/or filtration 600.

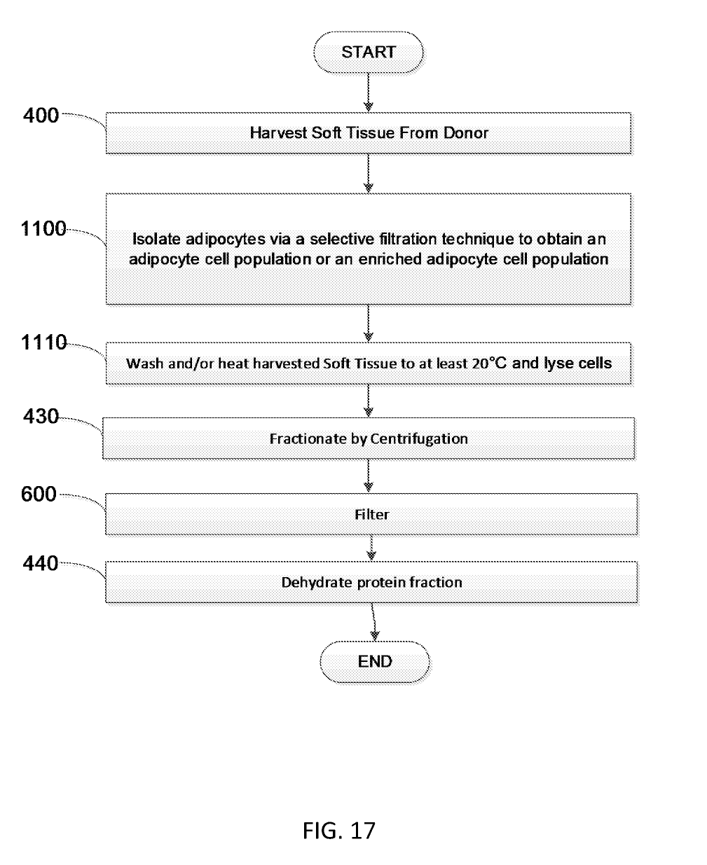

In some embodiments, it can be desirable to obtain proteins or bioactive factors specifically from adipocytes or other specific soft tissue cell type. As shown in FIG. 16, soft tissue can be harvested from a donor 400 as previously described in reference to harvesting soft tissue 400 in FIG. 9. Adipocytes or other soft tissue cell can be isolated via a selective filtration technique to generate an adipocyte (or other soft tissue cell) population or an enriched adipocyte (or other soft tissue cell) population 1100. In some embodiments, the resulting adipocyte or other soft tissue cell population is about 50% to about 100% adipocytes or soft tissue cell. The harvested soft tissue or isolated soft tissue cells can be washed and lysed 1110 as previously described in reference to FIG. 9, step 410. Suitable selective lysing techniques are described elsewhere herein, for example, in reference to FIG. 13. In some embodiments, heating is sufficient to separate adipocytes or other specific soft tissue cell from other undesired cells in the harvested soft tissue to obtain the desired adipocyte (or other soft tissue cell) cell population.

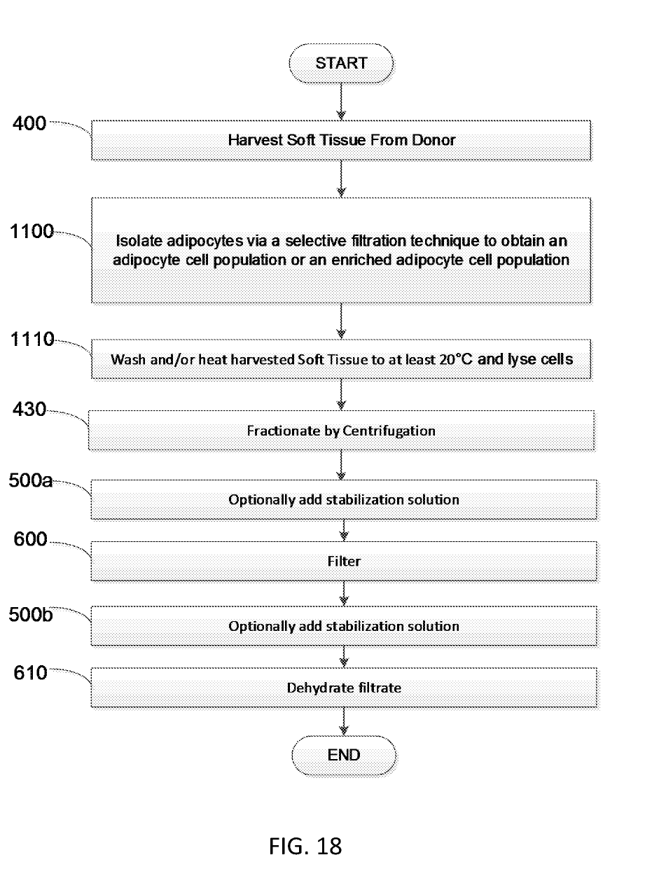

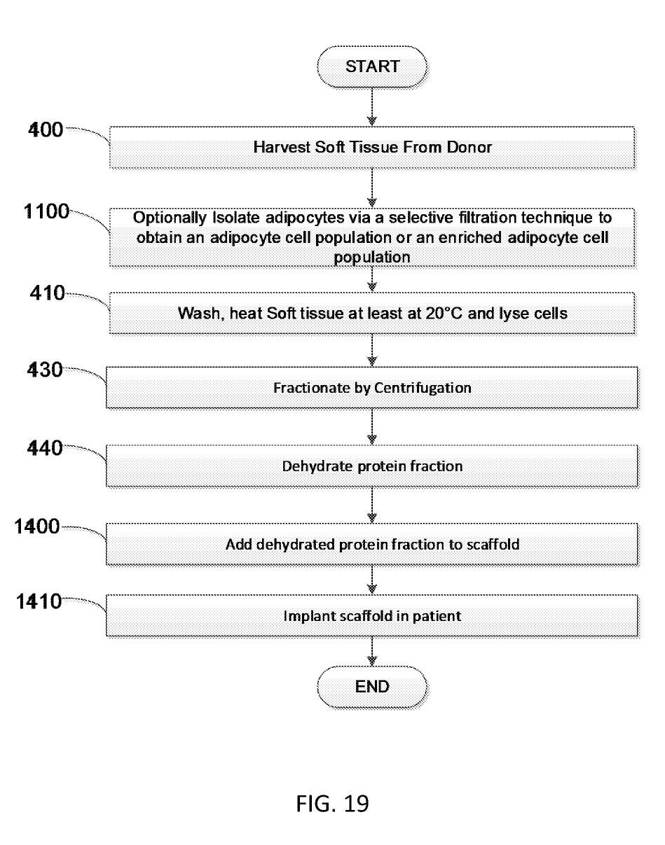

The adipocytes (or other soft tissue cell) or the cell population enriched for adipocytes (or other soft tissue cell) can be lysed 1110 to obtain adipocyte or primarily adipocyte derived proteins and/or other bioactive factors. As previously described, the lysate can be fractionated by centrifugation 430 and the desired proteins and/or bioactive factor containing fraction can be dehydrated 440 as previously described. As shown in FIGS. 17 and 18, the method can include the optional steps of filtering 600 after optional centrifugation 430 and/or adding a stabilizer 500 a,b after the step of optionally centrifuging 430 and/or filtering 600.

It will be appreciated that other steps can be included in any of the methods described herein. In some embodiments, the method can include a pH altering step where an acid, base and/or an acidic or basic solution can be added to product of any step in any method to result in a product that is acidic (pH less than 7), basic (pH greater than 7), or neutral (pH of 7). In some embodiments, after lysing, the lysate or product from any other subsequent step can be made more acidic, neutral, or basic as desired. In embodiments, the dehydrated product containing the soluble soft tissue derived proteins and/or bioactive factor(s) contains an acid that was introduced in the lysing step (e.g. 410, 810, or 1110). In other embodiments, the stabilization solution can contain an acid or base that can result in an acidic, basic, or neutral solution.

In some embodiments, the method can include a concentration step, where the product of any step in any embodiment of the method can be concentrated by a suitable concentration technique. Suitable concentration techniques include but are not limited to, dehydration techniques (described elsewhere herein) and centrifugation based techniques. Other concentration techniques will be appreciated by those of skill in the art.

Soluble Soft Tissue Protein Compositions

The soft tissue protein compositions can be harvested according to a method described herein from a suitable soft tissue. As used herein, “soft tissues” includes any tissue except for bone and bone marrow, and includes, but is not limited to, adipose tissue, muscle, cartilage, skin, tendons, ligaments, fascia, skin, fibrous tissue, synovial membranes, connective tissue, nerves, blood vessels, blood, lymph, and any organ.

The soluble bone soft tissue compositions can contain proteins and/or other non recombinant bioactive factors derived from cells present in the soft tissue, including but not limited to stem cells, adipocytes, myoblasts, myotubes, myocytes, chondroblasts, chondrocytes, fibroblasts, ganglion, nerve cells, glial cells (including macroglia and microglia), Schawann cells, astrocytes, oligodendrocytes, skin cells (e.g. keratinocytes, melanocytes, Merkel's cells, Langerhans' cells, stratum basale cells, prickle cells, and epithelial cells). The proteins can be intracellular proteins or membrane associated proteins. Such proteins include without limitation, bone morphogenetic proteins (BMPs) (e.g. BMP-1, BMP-2, BMP-3, BMP-4, BMP-5, BMP-6, BMP-7, BMP-8a, BMP-8b, BMP-10, and BMP-15), epidermal growth factor (EGF), insulin-like growth factors (IGFs) (e.g. IGF-1), fibroblast growth factors (FGFs) (e.g. αFGF (acidic fibroblast growth factor) and bFGF (basic fibroblast growth factor)), vascular endothelial growth factor (VEGF) osteoprotegerin (OPG), osteopontin (OPN), adipokines (e.g. resistin, adiponectin, leptin and apelin), fibrin, fibrinogen, other blood clotting factors (e.g. Factors), albumins, gloubulins, protein hormones, cytokines, chemokines, and nerve growth factorβ (NGFβ).

The soluble soft tissue protein composition can be 0% to 100% dehydrated. In some embodiments the soluble soft tissue protein composition can be about 100% dehydrated. The soluble soft tissue protein compositions do not inherently contain recombinant proteins. In some embodiments, the soluble bone soft tissue composition can be liquid or flowable solution. In some embodiments, the soluble soft tissue protein composition is frozen. The concentration of one or more of the bioactive factors in the soluble soft tissue protein compositions can be present in the composition at a concentration greater than or less than would be found in a cell within the body. The soluble soft tissue protein composition(s) as described herein can increase the efficiency of implant and/or graft integration and/or healing over that of the proteins if present in the context of complete soft tissue or other complete bodily fluid or tissue.

Additionally, the soluble soft tissue protein composition(s) described herein can lack the immunogenic proteins and other components that are present in complete soft tissue and/or other complete bodily fluid or tissue. The soluble soft tissue protein compositions provided herein, in some embodiments, do not include a recombinant or synthetic protein or other bioactive factor. In other words, in some embodiments the soluble soft tissue protein composition can be a non-recombinant soluble soft tissue protein composition.

Any given soft tissue protein and/or other bioactive factor can be present in the soluble soft tissue protein composition at a concentration of 0 pg/g to about 100 mg/g of isolated protein in the final product, dehydrated or otherwise provided.

Additionally, the soluble soft tissue protein composition can also contain an amount of a suitable acid. In some embodiments, the acid is a residual or other amount of the acid that can be used to lyse the soft tissue cells. In some embodiments, the acid can be acetic acid. Other suitable acids are described elsewhere herein. The acid can facilitate and/or increase binding of the proteins in the soluble soft tissue protein composition to a scaffold or other bodily tissue when the proteins are diluted or rehydrated during use, which is described elsewhere herein.

In some embodiments, soluble soft tissue protein composition can include a stabilizer composition or stabilizer compounds. Suitable stabilization compounds can include, but are not limited to preservatives, antibiotics, antivirals, antifungals, pH stabilizers, osmostablizers, anti-inflammants, anti-neoplastics, chemotherapeutics, immunomodulators, chemoattractants, growth factors, anticoagulants, or combinations thereof. The stabilization solution can increase shelf life of the soft tissue soluble protein composition and/or reduce denaturation of proteins during dehydration, sterilization, and/or storage. In addition, other materials, such as nitrogen, can be used to help reduce free radical formation and denaturation during sterilization. In some embodiments, the stabilization solution per cc of final product can be about 1 mg Sucrose, about 5 mg Glycine, about 3.7 mg I-Glutamic Acid, about 0.02 mg NaCl, and about 0.02 mg Polysorbate-80.

In some embodiments, a dehydrated or liquid soluble soft tissue protein composition can be reconstituted. This can result in a dilution of the bioactive factors within the dehydrated soluble soft tissue composition. In some embodiments, dehydration of a liquid soluble soft tissue protein composition can be dehydrated, which can result in a concentration of the proteins in the composition. The soluble soft tissue protein composition can be diluted/concentrated from 0.1 to 100 fold, 0.1 to 50 fold, 0.1 to 20 fold, or 0.1 to 5 fold.

In some embodiments, the final volume of a reconstituted or a liquid soluble soft tissue protein composition can be at least 1 cc, or 1 cc to about 100 cc, about 1 cc to about 50 cc, 1 cc to about 25 cc, about 1 cc to about 20 cc, about 1 cc to about 10 cc. The final soluble soft tissue protein product can be dehydrated or reconstituted to achieve a desired volume or particular protein concentration or composition.

Methods of Using the Soluble Soft Tissue Protein Compositions

The soluble soft tissue protein compositions (dehydrated or otherwise formulated as described herein) can contain an acid or be at an acidic pH. The soluble soft tissue protein compositions can be implanted into or otherwise administered to a subject in need thereof. In some embodiments, an effective amount of the soluble soft tissue protein composition (dehydrated or otherwise formulated) can be implanted or otherwise administered to a subject in need thereof. When implanted or administered, the soft tissue proteins and/or other bioactive factors and the acid can be diluted and/or reconstituted by the bodily fluids of the subject. When this occurs, an acid microenvironment surrounding the proteins and/or other bioactive factors can be created. The acidic microenvironment surrounding the soluble soft tissue protein composition can facilitate solublization of the soft tissue derived proteins and/or other bioactive factors in the composition and can also facilitate the binding of the soft tissue proteins and/or other bioactive factors a scaffold (natural or synthetic), bone, cartilage, or other tissue of the subject at the site where the soluble soft tissue protein composition is deposited within the subject.

The soluble soft tissue protein compositions (dehydrated or otherwise formulated as described herein) can be added to a suitable scaffold or device. Suitable scaffolds include, but are not limited to, allogeneic, autologous, syngeneic, or xenogeneic complete extracellular matrix, decellularized or acellular extracellular matrix, or extracellular matrix components, hydrogels, synthetic or natural polymer solids and semi-solids, carbohydrates, self-assembling peptides, carbon nanotubes, chitosan, alginate, hyaluronic acid, bone powder, cartilage powder, proteins, sugars, plastics, metals, or combinations thereof. In some embodiments, the scaffold can be biocompatible. In other embodiments, the scaffold can be allogeneic, xenogenic, or autologous bone or demineralized bone. The scaffold can be flowable or non-flowable.

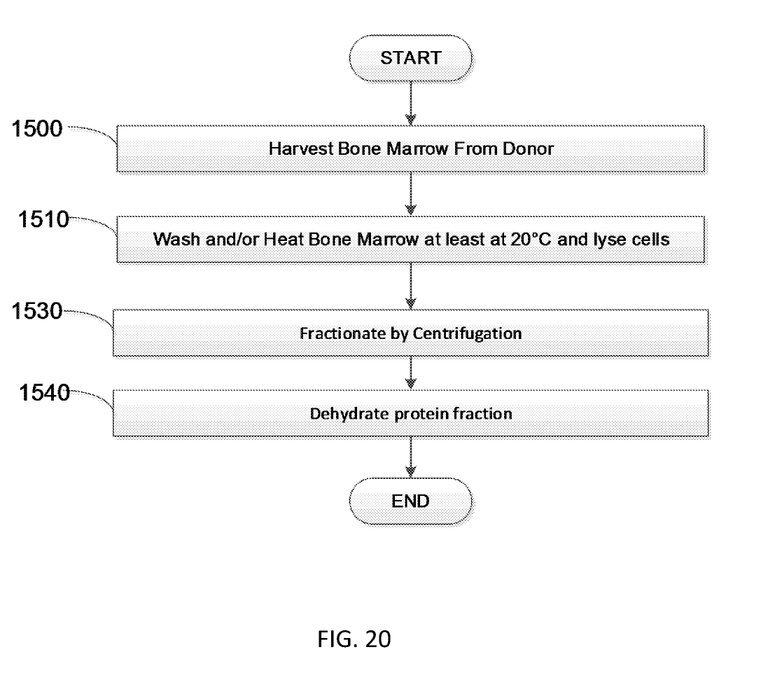

In some embodiments, the soluble soft tissue protein composition can be implanted or otherwise administered to a subject in need thereof without a scaffold material. In other embodiments, as shown in FIG. 19, the soluble soft tissue protein composition can be applied to a scaffold (implant) 1400, which is already present in a subject or can be implanted into a subject in need thereof 1410. When implanted 1410, the proteins in the dehydrated (or otherwise formulated) soluble soft tissue derived protein composition can solubilize and/or bind the scaffold when they come in contact a bodily fluid present in the subject. The acid present in the dehydrated (or otherwise formulated) soluble soft tissue derived protein composition can create an acidic microenvironment where the scaffold and/or soluble soft tissue protein composition is present. The acidic microenvironment can facilitate solubilization of the soft tissue derived proteins and/or binding of the proteins and/or other bioactive factors to the scaffold (synthetic or natural) and/or other bone or tissue of the subject that are at the site of implantation. In some embodiments, the soluble soft tissue protein composition can be added in a dehydrated state to an implant material to encapsulate the proteins such as a putty, gel, or suspension.

In other embodiments, the soluble soft tissue derived protein composition can be applied directly into a scaffold already present in the subject in need thereof. As previously described, the proteins and/or other bioactive factors can be diluted or reconstituted when contacted with a bodily fluid present within the subject. As also described above, the acid that can be present in the soft tissue protein compositions described herein can create an acidic microenvironment that can facilitate solubilization and/or binding of the soft tissue proteins and/or bioactive factors to a scaffold present in the subject.

In some embodiments, the method can include the step of implanting or otherwise administering a soluble soft tissue protein composition or scaffold incorporating a soluble soft tissue protein composition as described herein to a subject in need thereof. In some embodiments, a method of treating a subject in need thereof can include the step of implanting or otherwise administering a soluble soft tissue protein composition or scaffold incorporating a soluble soft tissue protein composition as described herein to the subject in need thereof. In some embodiments, the subject in need thereof needs a soft tissue graft or soft tissue augmentation.

Soluble Bone Marrow Protein Compositions and Methods of Making

Bone grafting is a common procedure performed for a variety of orthopedic and dental reasons. Many materials have been developed that can be used for bone graft procedures. Such materials include, but are not limited to, autograft, allograft, and synthetic bone graft materials. While these materials have enjoyed a certain amount of clinical success, donor morbidity when using autograft materials, adverse recipient immune response when using allograft materials, and limited bone remodeling and low osteoconductivity that can be observed when using synthetic materials. Attempts to improve the clinical performance of all types of materials have employed the use of recombinant or synthetic bioactive factors that are involved in the bone-remodeling process. While there have been attempts to obtain bioactive factors directly from various tissue sources, all have relied upon harsh chemicals to isolate the bioactive factors, which can lead to low yields of viable bioactive factors such and reduce clinical performance of the bioactive factors obtained. Further, the variability in the amount and type of bioactive factors obtained directly from tissue sources due to the methods used to obtain the bioactive factors severely limits this approach for any practical clinical purpose.

With the aforementioned shortcomings in mind, described herein are soluble bone marrow protein compositions and scaffolds that can include a soluble bone marrow protein composition provided herein. The soluble bone marrow protein composition can be a non-recombinant soluble bone marrow protein composition. The soluble bone marrow protein compositions provided herein can, in some embodiments, overcome one or more of the shortcomings of existing soluble bone marrow compositions and graft scaffold materials. Other compositions, compounds, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

Soluble Bone Marrow Protein Compositions and Scaffolds

Soluble Bone Marrow Protein Compositions

Bone marrow is the soft, spongey, gelatinous tissue found in the hollow spaces in the interior of bones. Bone marrow contains stem cells that are supported by a fibrous tissue called the stroma. There are two main types of stem cells in bone marrow: (1) hematopoietic stem cells and (2) bone marrow mesenchymal stem cells (bmMSCs). bmMSCs can differentiate into a variety of cells types including without limitation, fibroblasts, chondrocytes, osteocytes, myotubes, stromal cells, adipocytes, astrocytes, and dermal cells. In addition to bmMSCs, bone marrow stroma contains other types of cells including fibroblasts (reticular connective tissue) macrophages, adipocytes, osteoblasts, osteoclasts, red blood cells, white blood cells, leukocytes, granulocytes, platelets, and endothelial cells.

The soluble bone marrow protein compositions can contain proteins and/or other non-recombinant bioactive factors derived from bone marrow mesenchymal stem cells, fibroblasts, chondrocytes, osteocytes, red blood cells, white blood cells, leukocytes, granulocytes, platelets, and/or osteoclasts. The proteins can be intracellular proteins or membrane associated proteins. Such proteins include without limitation, bone morphogenetic proteins (BMPs) (e.g. BMP-1, BMP-2, BMP-3, BMP-4, BMP-5, BMP-7 and BMP-8a), transforming growth factors (TGF-β1, TGF-β2), epidermal growth factor (EGF), hepatocyte growth factor (HGF), insulin-like growth factors (IGFs) (e.g. IGF-1), fibroblast growth factors (FGFs) (e.g. αFGF (acidic fibroblast growth factor) and bFGF (basic fibroblast growth factor)), vascular endothelial growth factor (VEGF), platelet derived growth factor-BB (PDGF-BB), osteoprotegerin (OPG), and osteopontin (OPN).

The soluble bone marrow protein composition can be 0% to 100% dehydrated. In some embodiments the soluble bone marrow protein composition can be about 100% dehydrated. In some embodiments, the soluble bone marrow protein composition can be liquid or flowable solution. In some embodiments, the soluble bone marrow protein composition is frozen. Techniques for freezing include slow and flash freezing in liquid nitrogen. The soluble bone marrow protein composition can be frozen to less than about 0° C. such as −10, −20, and −80° C. or more. The soluble bone marrow protein compositions do not inherently contain recombinant proteins. The concentration of one or more of the bioactive factors in the soluble bone marrow protein compositions can be present in the composition at a concentration greater than or less than would be found in a cell within the body. The soluble bone marrow protein composition(s) as described herein can increase the efficiency of implant and/or graft integration and/or healing over that of the proteins if present in the context of complete bone marrow or other complete bodily fluid or tissue.

Additionally, the soluble bone marrow protein composition(s) described herein can lack the immunogenic proteins and other components that are present in complete bone marrow and/or other complete bodily fluid or tissue. The soluble bone marrow protein compositions provided herein, in some embodiments, do not include a recombinant or synthetic protein or other bioactive factor. In other words, in some embodiments the soluble bone marrow protein composition can be a non-recombinant soluble bone marrow protein composition.

Any given bone marrow protein and/or other bioactive factor can be present in the soluble bone marrow protein composition at a concentration of 0 pg/g to about 100 mg/g of isolated protein in the final product, dehydrated or otherwise provided. The soluble bone marrow protein composition can include at least 1 pg/g, about 1 pg/g to about 100 pg/g, about 7 pg/g to about 100 pg/g, or about 7 pg/g to about 35 pg/g BMP-2 protein derived directly from bone marrow. The soluble bone marrow protein composition can include at least about 1 pg/g αFGF, about 1 pg/g to about 100 pg/g αFGF, about 1 ng/g to about 100 ng/g αFGF, or about 20 to about 40 ng/g αFGF. The soluble bone marrow protein composition can include at least about 1 pg/g bFGF, about 1 pg/g to about 100 pg/g bFGF, about 1 ng/g to about 100 ng/g bFGF, or about 20 ng/g to about 40 ng/g bFGF. The concentration of VEGF in the soluble bone marrow protein composition can be at least about 1 pg/g, or about 1 pg/g to about 100 pg/g VEGF, about 1 ng/g to about 150 ng/g VEGF, or about 60 ng/g to about 90 ng/g VEGF. The soluble bone marrow protein composition can include at least 1 pg/g PDGF, or about 1 pg/g PDGF to about 100 pg/g PDGF, about 500 pg/g to about 500 ng/g PDGF, about 900 pg/g to about 100 ng/g PDGF, or to about 950 pg/g to about 50 ng/g PDGF. The soluble bone marrow protein composition can include at least 1 pg/g OPN, or about 1 pg/g OPN to about 100 pg/g OPN, about 500 pg/g OPN to about 500 ng/g OPN, about 900 pg/g to about 100 ng/g OPN, or to about 950 pg/g to about 50 ng/g OPN

Additionally, the soluble bone marrow protein composition can also contain an amount of a suitable acid. In some embodiments, the acid is a residual or other amount of the acid that can be used to lyse the bone marrow cells. In some embodiments, the acid can be glutamic acid or acetic acid. Other suitable acids are described elsewhere herein. The acid can facilitate and/or increase binding of the proteins in the soluble protein composition to a scaffold when the proteins are diluted or rehydrated during use, which is described elsewhere herein.

In some embodiments, soluble bone marrow protein composition can include a stabilizer composition or stabilizer compounds. Suitable stabilization compositions can include, but are not limited to preservatives, antibiotics, antivirals, antifungals, pH stabilizers, osmostablizers, anti-inflammants, anti-neoplastics, chemotherapeutics, immunomodulators, chemoattractants, growth factors, anticoagulants, or combinations thereof. The stabilization solution can increase shelf life of the soft tissue soluble protein composition and/or reduce denaturation of proteins during dehydration, sterilization, and/or storage. In addition, other materials, such as nitrogen, can be used to help reduce free radical formation and denaturation during sterilization. In some embodiments, the stabilization solution per cc of final product can be about 1 mg Sucrose, 5 mg Glycine, 3.7 mg I-Glutamic Acid, 0.02 mg NaCl and 0.02 mg Polysorbate-80.

In some embodiments, a dehydrated or liquid soluble bone marrow protein composition can be reconstituted. This can result in a dilution of the bioactive factors within the dehydrated soluble bone marrow composition. In some embodiments, dehydration of a liquid soluble bone marrow protein composition can be dehydrated, which can result in a concentration of the proteins in the composition. The soluble bone marrow protein composition can be diluted/concentrated from 0.1 to 100 fold, 0.1 to 50 fold, 0.1 to 20 fold, or 0.1 to 5 fold.

In some embodiments, the final volume of a reconstituted or a liquid soluble bone marrow protein composition can be at least 1 cc, or 1 cc to about 100 cc, about 1 cc to about 50 cc, 1 cc to about 25 cc, about 1 cc to about 20 cc, about 1 cc to about 10 cc. The final soluble bone marrow protein product can be dehydrated or reconstituted to achieve a desired volume or particular protein concentration or composition.

Scaffolds Including a Soluble Bone Marrow Derived Composition

Many suitable graft scaffold materials are known in the art and can include those from autograph, allograft and synthetic sources. CORTOSS® bone augmentation material is a synthetic, injectable, non-resorbable, polymer composite that mimics cortical bone. CORTOSS® bone augmentation material is a self-setting glass ceramic polymeric composite engineered specifically to mimic the characteristics of human bone and can provide fixation for vertebral compression fractures (“VCFs”). Laboratory tests demonstrate that CORTOSS® bone augmentation material can exhibit compressive strength similar to human bone.

VITOSS® bone graft substitute material is a synthetic, ultra-porous resorbable beta-tricalcium phosphate bone void filler that can be used to help the subject's body guide the three-dimensional regeneration of the patient's own bone. VITOSS® bone graft substitute material's ultra-porosity can allow it to soak and hold up to its own volume of other compositions. VITOSS® bone graft substitute material integrates well into existing bone and can allow for bone in-growth and maturation. VITOSS® bone graft substitute material can be provided in a variety of platforms including, but not limited to, blocks, chips, morsels (micro and macro) canisters (micro and standard), foam (strips, cylinders, flow, shapes, and packs), cement (e.g. a bone graft cement) and bioactive foam (strips and packs). VITOSS® foam-based bone graft materials combine the base VITOSS® material technology with resorbable biomaterials to produce a wide array of pliant, flexible, flowable and compression resistant bone graft materials. The cement can exhibit exothermic properties that result in burning of tissues such as nerves in the area surrounding the implant and in some instances improve the clinical outcome and/or recovery of the recipient. The VITOSS® foam-based bone graft materials can soak and hold their own volume in other compositions (e.g. blood and bone marrow aspirate while retaining these biological fluids in pliable and compression resistant forms. These forms can be designed into specific shapes and material characteristics to meet a surgeon's need for handling and delivery in a variety of surgical approaches and applications.

VITOSS® Boactive bone graft substitute materials also contain bioactive glass. Upon implantation, the ionic constituents (e.g. Si+, Na+, Ca²⁺) of bioactive glass can be released into the surrounding environment and can react with bodily fluids. This reaction can produce the deposition of a thin layer of physiologic CaP at its surface. This can attract osteoblasts to the layer to create a matrix that can produce an osteostimulatory effect. This can lead to the bonding of new bone to the scaffold.

As previously discussed, the VITOSS® and CORTOSS® synthetic scaffolds have been described to be supplemented with autologous and allogeneic whole bodily fluids and tissue such as blood and/or bone marrow aspirate. Currently, scaffold materials, including VITOSS® and CORTOSS® synthetic scaffolds, have been combined only with recombinant proteins.

Provided herein are grafting scaffold materials (also referred to herein as “scaffolds”) that can include a soluble bone marrow protein composition provided elsewhere herein that can have one or more proteins of the composition bound adsorbed, absorbed, or is otherwise attached to or associated with a scaffold material. Described herein are embodiments of scaffolds, including VITOSS® and CORTOSS® materials, biopolymers, collagen, chitosan, alginate, calcium phosphate, calcium sulfate, or any combinations thereof further containing a soluble bone marrow protein composition described herein.

The soluble bone marrow protein composition can be any soluble bone marrow protein composition provided herein. The soluble bone marrow protein composition including or not including the scaffold material can be a 0% to 100% dehydrated. The soluble bone marrow composition, proteins and/or other bioactive factor(s) can become solubilized and/or reconstituted when contacted with bodily fluids, for example, when the VITOSS® material, CORTOSS® material, and/or other scaffold material containing the soluble bone marrow protein composition are implanted in or otherwise administered to a subject in need thereof. As described elsewhere herein, the soluble bone marrow protein composition can contain an amount of an acid. The acid can be acetic acid, formic acid, trichloroacetic acid, hydrofluoric acid, hydrocyanic acid, hydrogen sulfide, or hydrochloric acid. The acid can be a residual amount left over from the method of producing the soluble bone marrow composition. The acid can facilitate and/or increase the binding and/or retention of the protein(s) and/or other bioactive factors in the soluble bone marrow protein composition bind to or otherwise be attached to or associated with the scaffold material.

Scaffold Materials

The scaffold material can be as described in U.S. Pat. Nos. 5,681,872; 5,914,356; 5,939,039; 6,325,987; 6,383,519; 6,521,246; 6,736,799; 6,800,245; 6,969,501; 6,991,803; 7,052,517; 7,189,263; 7,534,451; 8,303,967; 8,460,686; 8,647,614, which are incorporated by reference herein as if expressed in their entirety. Other suitable scaffold materials include biopolymers, bone, decellularized bone, extracellular matrix or components thereof, fibrin collagen, chitosan, alginate, calcium phosphate, calcium sulfate, poly(alpha-hydroxy acids) such as poly(lactic-co-glycolic acid) and polyglycolic acid, CUPE polymer, polyethylene glycol, or any combinations thereof. The scaffold material can be porous. The scaffold material can be a natural material, synthetic material, or a combination thereof. The scaffold material can be biocompatible, nontoxic, and/or non-inflammatory. The scaffold material can support cell attachment, cell proliferation, extracellular and/or bone matrix production, and/or cell differentiation. The scaffold material can be biodegradable. The scaffold material can be sterilized. Other scaffold materials and attributes will be appreciated by those of skill in the art in view of the discussion provided herein.

Methods of Making the Soluble Bone Marrow Protein Compositions

Described herein are methods for producing compositions containing soluble bone marrow proteins and/or other bioactive factor(s). The methods described herein can also result in a composition containing a dehydrated soluble bone marrow protein(s) and/or other bioactive factor(s). In some embodiments, the dehydrated soluble bone marrow protein(s) and/or other bioactive factor(s) can bind to a scaffold upon reconstitution, or encapsulated prior to delivery, such as when the dehydrated soluble protein composition comes in contact with a bodily fluid, solution containing water, or saline. The soluble protein compositions prepared by the methods described herein can have a greater amount and/or concentration of bone marrow protein(s) and/or additional bioactive factor(s), and/or less immunogenicity than other osteoinductive/osteostimulatory compositions, implants, or devices incorporating complete bone marrow and/or other complete bodily fluids or tissues. The soluble bone marrow protein compositions can contain bioactive proteins such as, but not limited to, BMP-2, acidic-FGF, basic-FGF, IGF, BMP-7, HGF, VEGF, PDGF-BB, OPG, and OPN.

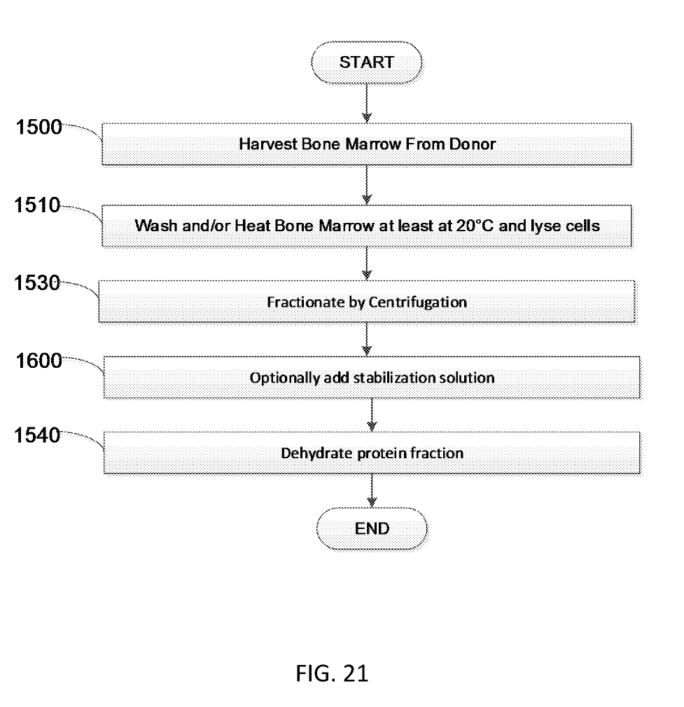

Attention is first directed to FIG. 20, which shows an embodiment of a method of producing a soluble protein composition from bone marrow. The bone marrow can be harvested from a cadaver or from a living subject. The method can begin by harvesting bone marrow from a donor 1500. The donor can be a cadaver or can be a living subject. The bone marrow can be autologous, allogeneic or xenogenic. The bone marrow can be harvested in any way generally known in the art. The bone marrow can be obtained from cancellous, corticocancellous, and/or cortical bone. The harvest of the bone marrow may also include bone prior to washing. After the bone marrow has been harvested, the bone marrow is washed 1510 in a solution. The wash solution may contain water, saline, antibiotic, antiseptic, antifungal, or crystalloid solution. In some embodiments, the wash solution is only water. Washing can take place at least at 20° C. In some embodiments, washing takes place at about 20° C. to about 37° C. In further embodiments, washing takes place at about 20° C. to about 40° C. In some embodiments, the washing takes place at 37° C. Heating the bone marrow during washing facilitates the reduction in viscosity or removal of undesired fat (adipocytes) from other types of bone marrow cells. The washing/heating step can be performed under physical agitation in a shaker incubator. In some embodiments, shaking ca be conducted at about 10-300 rpm for up to about 24 hours. In some embodiments, shaking can be conducted for about 20, 40, 60, 120, 240, 260, 5, 6, 7, 8, 9, 10, 11, 12, 13, 14, 15, 16, 17, 18, 19, 20, 21, 22, 23 or 24 hours.

During washing/heating 1510, the bone marrow derived cells can be lysed. In some embodiments, the bone marrow derived cells can be lysed using a lysing solution containing water, salt, or an acid. In some embodiments, the lysing solution is just water. In some embodiments, the washing solution and the lysing solution can be the same solution. The acid can be acetic acid, formic acid, trichloroacetic acid, hydrofluoric acid, hydrocyanic acid, hydrogen sulfide, or hydrochloric acid. In some embodiments, the lysis solution contains about 0.001M to about 1M acetic acid. In some embodiments the lysing solution that contains the bone marrow and/or marrow-rich bone is mixed with pre-heated water. In some embodiments, the bone marrow or marrow-rich bone is lysed for about 60 minutes. In other embodiments, the bone marrow or marrow-rich bone is incubated in the lysing solution with shaking. In other embodiments, the lysing conditions can include, but are not limited to, ultrasonic techniques, thermolysis (e.g. freeze/thaw cycling), microfluidic techniques, osmotic shock, electric shock, homogenization, French press, impingement, excessive shear (e.g. aggressive pipetting through a small aperture, centrifuging at excessive revolutions per minute resulting in high gravity forces), pressure, vacuum forces, milling or bead beating techniques that physically collide or grind cells to mechanically break cell membranes, pH shock, exposure to detergents, enzymes, viruses, solvents, surfactants, hemolysins, or combinations thereof.

After washing/lysing 1510, the lysate can be optionally fractionated via centrifugation 1530 to separate out particles present in the lysate based on their size or density. Such centrifugation techniques that can be employed include, but are not limited to, differential centrifugation, rate-zonal centrifugation, and isopycnic centrifugation. In embodiments where centrifugation is used to separate particles in the lysate based on density, a suitable density gradient medium can be used. Suitable density gradient mediums include, but are not limited to, sucrose, glycerol, sorbitol, Ficoll® medium, polysucrose, dextrans, CsCl, Cs₂SO₄, KBr, Diatrizoate, Nycodenz® medium, Histodenz™ medium, iodixanol, Histopaque® mediums, ACCUSPIN® medium, and Percoll® medium. One of ordinary skill in the art will appreciate that the type of medium used is dependent on the type of particle(s) that is desired to be separated out. One or more rounds of centrifugation can be applied to the lysate to further separate out different particles in the lysate. In some embodiments, the desired fraction contains a bioactive factor, such as, but not limited to, a cytokine or osteoinductive protein. In some embodiments, the lysate is centrifuge at about 100 to about 20000 rpm for about 1 to about 600 minutes. In some embodiments, the lysate is centrifuged at about 4000×g for about 10 minutes at about 4° C.

After the optional fractionation 1530, the desired fraction can be removed from the centrifuged lysate. In some embodiments, the desired fraction contains one or more bioactive factor, such as, but not limited to, a cytokine or osteoinductive protein. The bioactive factor containing fraction can then be dehydrated 1540 using a suitable technique. Suitable dehydrating techniques include, but are not limited to, evaporation, vacuum drying, lyophilization, freeze drying, sublimation, and precipitation. After dehydration, the soluble protein composition can contain an acid, such as glutamic acid, that can be reconstituted along with the proteins and other bioactive factors that can be present in the soluble protein composition.

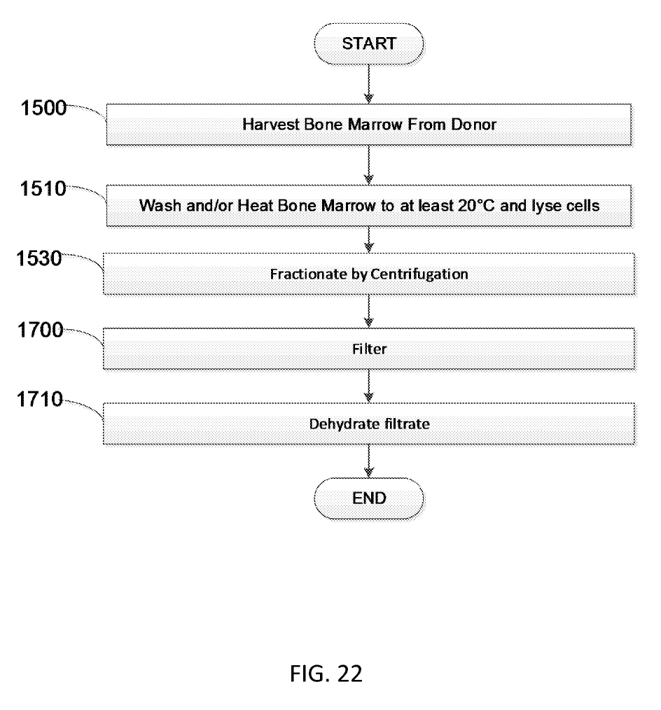

With the general process described, attention is directed to FIGS. 21-29, which demonstrate various embodiments of the general method of producing a soluble bone marrow derived soluble protein composition. Discussion begins with FIG. 21, which demonstrates embodiments of a method of generating a soluble bone marrow derived protein composition. As in FIG. 20, bone marrow can be harvested 1500 and washed/heated and bone marrow derived cells can be lysed 1510. The desired components (e.g. bioactive factors) of the resulting lysate can be optionally separated from the undesirable components using by fractionating using a suitable centrifugation technique 1530. Once the desired fraction containing the proteins and/or bioactive factors of interest is obtained, the desired fraction can be dehydrated 1540 using a suitable dehydration technique. As shown in FIG. 21, an optional suitable stabilization solution can be added 1600 prior to dehydration 1540. Suitable stabilization solutions can aid in maintaining protein integrity and activity. In some embodiments, the stabilizer can include sucrose, trehalose, glycine, L-glutamic acid, sodium chloride, polysorbate-80 and combinations thereof. The stabilization solution can contain preservatives, antibiotics, antivirals, antifungals, pH stabilizers, osmostablizers, anti-inflammants, anti-neoplastics, chemotherapeutics, immunomodulators, chemoattractants, growth factors, anticoagulants, or combinations thereof. In some embodiments, the stabilization solution per cc of final product can be about 1 mg Sucrose, 5 mg Glycine, 3.7 mg I-Glutamic Acid, 0.02 mg NaCl and 0.02 mg Polysorbate-80.

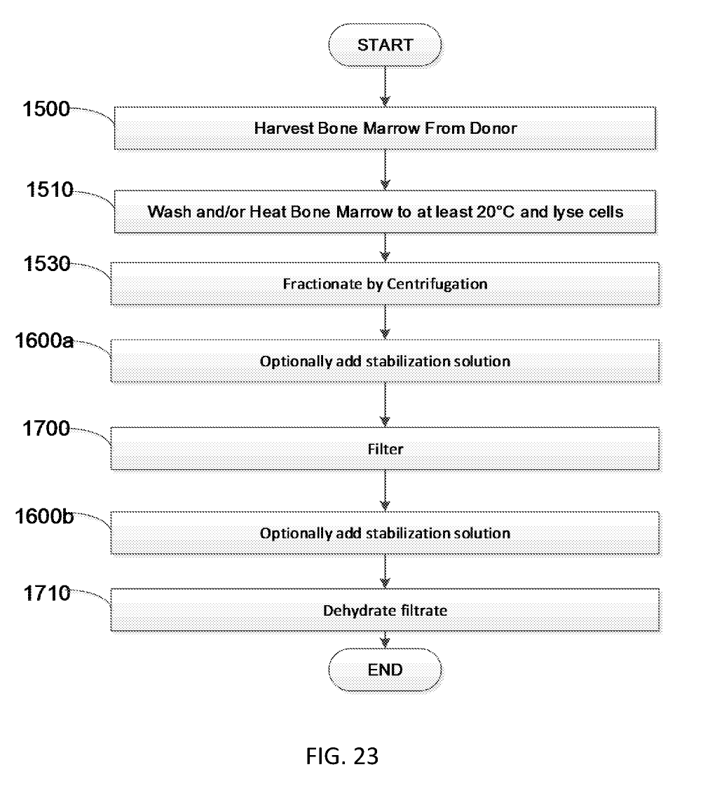

Discussion continues with FIG. 22, which shows another embodiment of a method of producing a soluble bone marrow derived soluble protein composition. As in FIG. 20, bone marrow can be harvested 1500 and washed/heated and lysed 1510. The desired components (e.g. bioactive factors) of the resulting lysate can be optionally separated from the undesirable components using by fractionating using a suitable centrifugation technique 1530. As shown in FIG. 22, after fractionation by centrifugation 1530 the fraction containing the desired components can be further filtered using a suitable filtration technique to remove additional undesired components that can remain in the fraction. Suitable filtration techniques can include, but are not limited to, size exclusion techniques and/or affinity purification techniques, immunoseparation techniques, and charged based separation techniques. In some embodiments, additional undesired components can include, but are not limited to, nucleic acids such as DNA and RNA, and other compounds such as hemoglobin, globin proteins, cell fragments, cell membrane molecules and other molecules that can stimulate an immune response in a subject. In some embodiments, the filter can be low protein binding. In some embodiments, the filter can be high DNA binding.

In some embodiments, the filter can preferentially bind one growth factor over another growth factor (such as, but not limited to, BMP-2, BMP-7, VEGF, αFGF, bFGF, IGF, HGF, or combinations thereof). Suitable materials for some filters used in the filtration step 300, include, but are not limited to, Teflon® membranes, nylon membranes, PVDF (polyvinylidene) membranes, polypropylene, cellulose acetate, PES (polyethersulfone), regenerated cellulose, glass fiber, and PTFE (polytetrafluorethylene. In some embodiments, the filter can have a size cutoff of about 0.1 to about 3.0 μM. In some embodiments multiple filters can be used, such as in a serial filtration system. In such a system, multiple types of filters can be used. The system can include at least two filters that differ in material and size cut offs. In some embodiments, polypropylene filters (e.g. size cut offs of 30 μm and 10 μm can be used), a glass fiber filter with a size cutoff of about 2.7 μm can be used, and/or a series of cellulose acetate filters (8 μm, 5 μm, 3 μm, 1.2 μm, 0.8 μm, 0.45 μm and final one of 0.2 μm) can be used to filter. The filters can be configured as syringe filters, disc filters, vacuum filter systems, bottle top vacuum filters, tube top vacuum filters, or centrifuge tube filters.

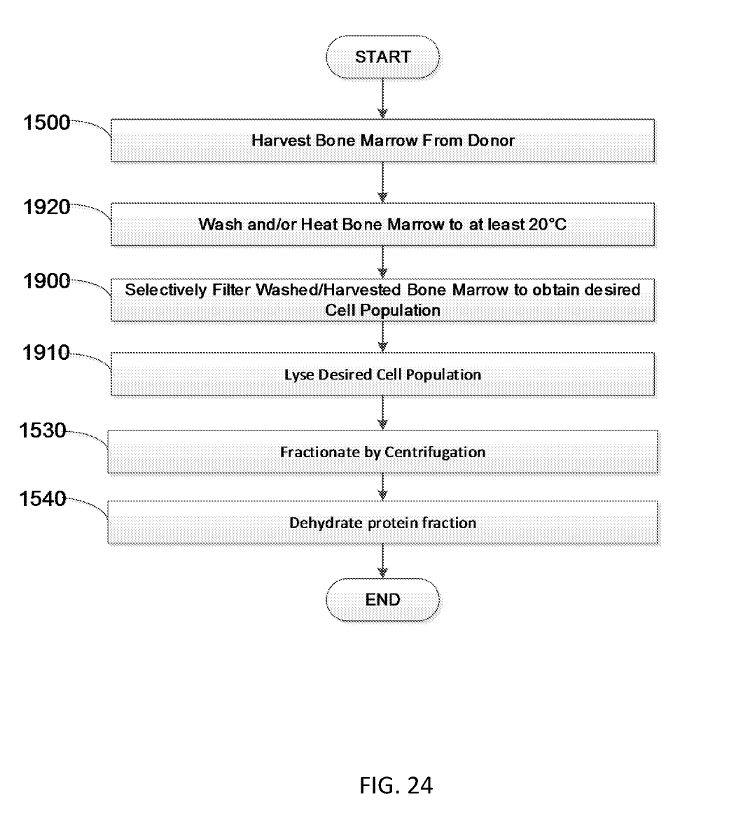

The filtrate obtained after filtering 1700 can contain the desired soluble bone marrow derived proteins. The filtrate can also contain acid that can be used during the lysing step 1510. The filtrate can be dehydrated 1710 using any suitable dehydration techniques. Suitable dehydration techniques are described with respect to dehydrating the protein fraction 1540 in FIG. 20. As shown in FIG. 23, an optional suitable stabilization solution can be added 1600 a,b to the product prior to dehydration 1710. The stabilization solution can be added after centrifugation 1530 and/or after filtration 1700. Suitable stabilization solutions are described elsewhere herein with respect to FIG. 21.

While the bone marrow can be heated 1510 to facilitate better penetration of lysing solution or viscosity reduction and/or removal of the undesired adipocytes that can be present in bone marrow tissue, in some instances it can be desirable to further filter the harvested bone marrow prior to or during lysing of the bone marrow desired cells.

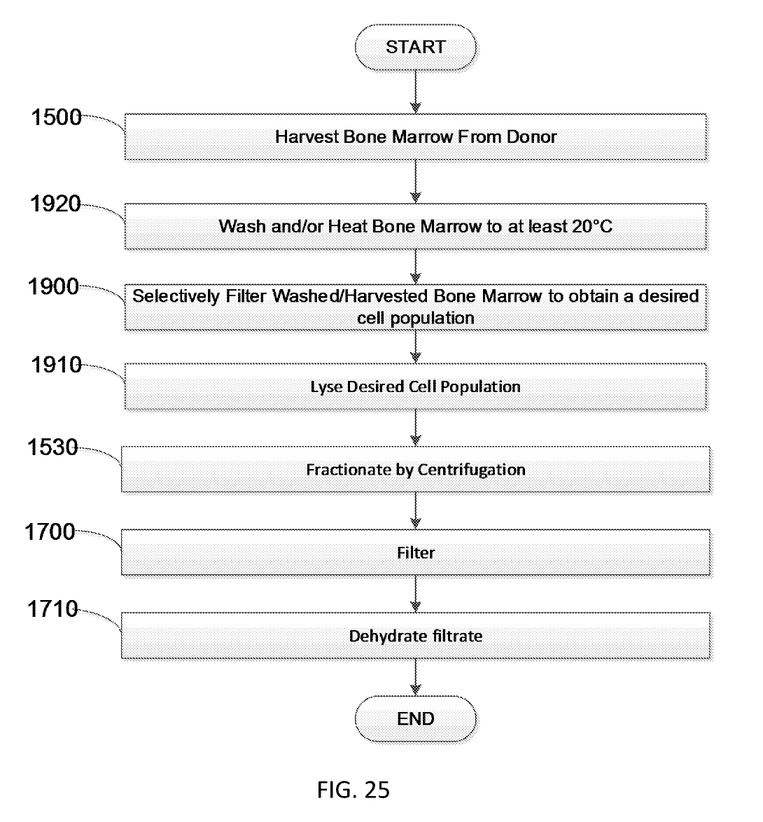

As shown in FIG. 24, bone marrow can be harvested 1500 from a donor as previously described in reference to FIG. 20. The harvested bone marrow can then be washed/heated 1920 as previously described with respect to FIG. 20. In some embodiments, the bone marrow cells are not all lysed during the washing step. The non-lysed cells can be further separated to obtain a desired cell population. The washed/heated bone marrow can then be selectively filtered to obtain a desired cell population 1900. Selective filtering can be completed by any suitable filtering techniques including, but not limited to, size exclusion separation techniques, affinity separation techniques, immunoseparation techniques, charge separation techniques, and chromatography techniques. For example, selective filtering can be achieved using osmotic lysis, cytolysis, centrifugation, size exclusion chromatography, ion exchange chromatography, expanded bed absorption chromatography, affinity chromatography (including but not limited to supercritical fluid chromatography), displacement chromatography, gas chromatography, liquid chromatography, column chromatography, planar chromatography (including, but not limited to paper chromatography, thin-layer chromatography), reverse-phase chromatography, simulated moving-bed chromatography, pyrolysis gas chromatography, fast protein liquid chromatography, high performance liquid chromatography, ultra-high performance liquid chromatography, countercurrent chromatography, chiral chromatography and solid phase extraction. In some embodiments, where bmMSC are desired, osmotic lysis can be used to select for bmMSC as they are resistant to cytolysis and osmotic lysis.

In some embodiments, the bone harvested bone marrow can be selectively filtered to obtain a desired cell population, such as bone marrow MSCs, prior to washing and lysing the bone marrow cells. In these embodiments, the washing and lysing can be performed under heating and can be as described as set forth in FIG. 20, step 1510.

After selective filtering of the bone marrow derived cells 1900 the remaining desired cell population is lysed 1910. Suitable lysing techniques are described with respect to FIG. 20. After lysing, the desired cell population can be fractionated 1530 by centrifugation as previously described with respect to FIG. 20. Finally the obtained desired fraction containing the desired bone-marrow derived proteins and/or other bioactive factors can be dehydrated as previously described with respect to FIG. 20.

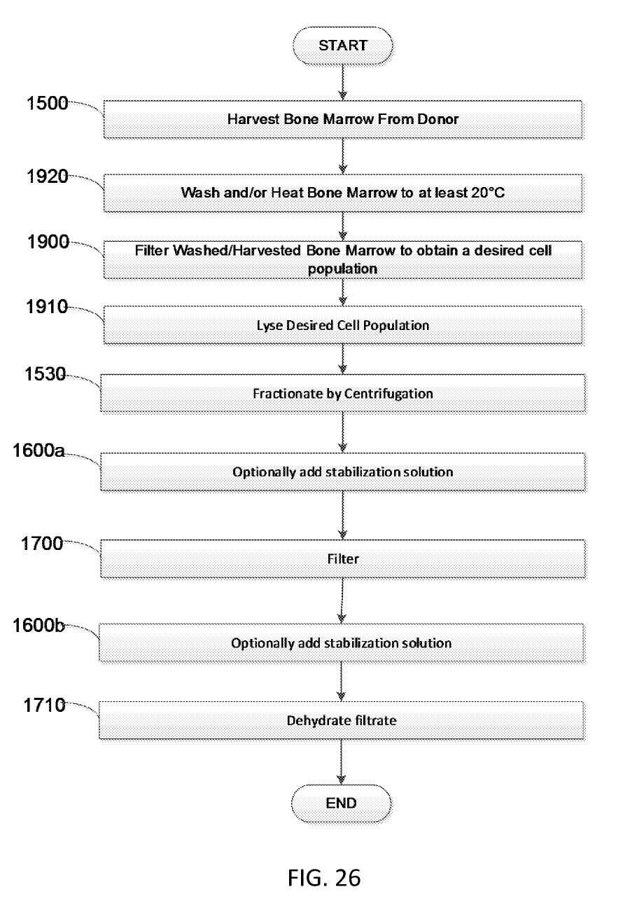

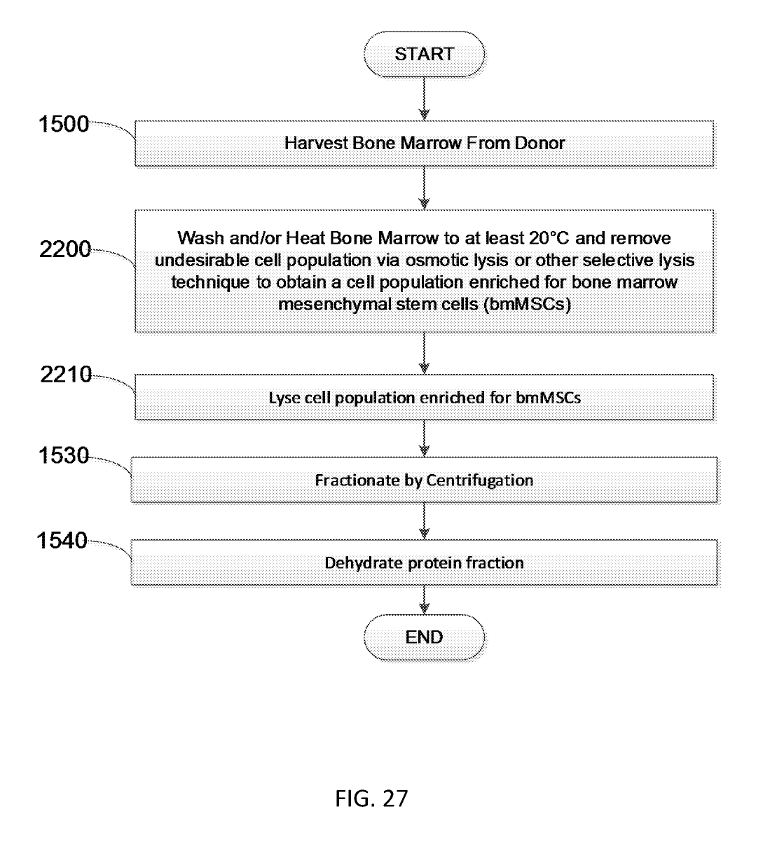

As shown in FIG. 25, the method where the harvested bone marrow can be selectively filtered 1900 prior during or prior to lysing (FIG. 24) can optionally include the step of filtering 1700 the obtained fraction after centrifugation 1530. Filtering 1700 can be performed as previously described with respect to FIG. 22. After filtering 1700, the desired filtrate can be dehydrated 1710 as previously described. As shown in FIG. 26, the methods (FIG. 24 and FIG. 25) where the harvested bone marrow can be selectively filtered 1900 prior to or during lysing can also include the optional step of adding a stabilization solution 1600 a,b after centrifugation 1530 and/or filtration 1700.

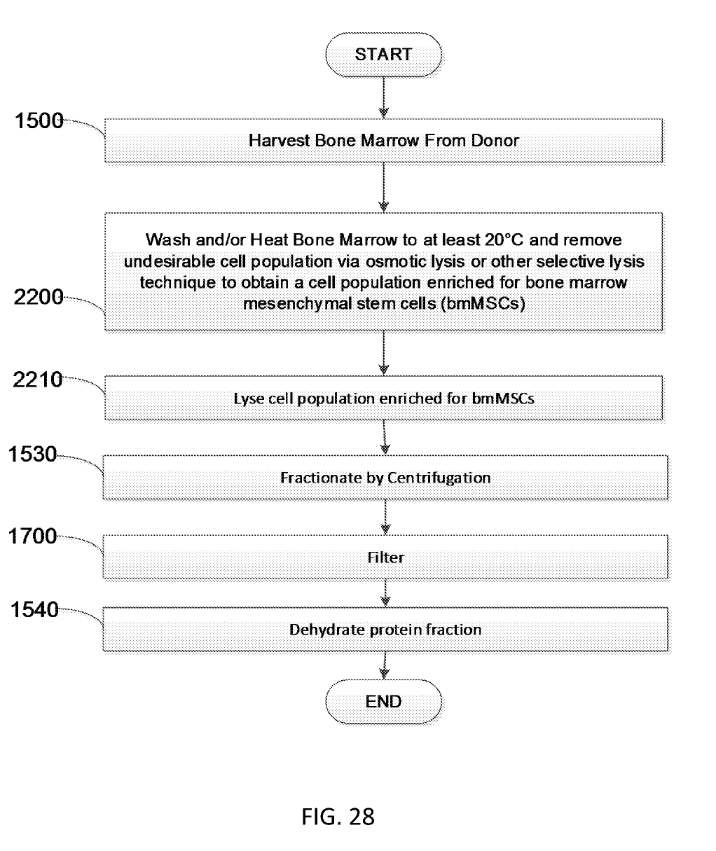

In some embodiments, it can be desirable to obtain proteins or bioactive factors specifically from bmMSCs. As shown in FIG. 27, bone marrow can be harvested from a donor 1500 as previously described in reference to FIG. 20. The harvested bone marrow can be washed and heated 1510 as previously described in reference to FIG. 20. After washing/heating the harvested bone marrow 1510, bmMSC can be separated from the undesirable cell population 2200 using osmotic lysis, cytolysis, or other suitable selective lysing technique to produce a population of cells that is completely bmMSCs or enriched for bmMSCs. Suitable selective lysing techniques are described elsewhere herein, for example, in reference to FIG. 24. As previously described, bmMSCs are resistant to osmotic lysis and cytolysis. As such after such treatments, most of the bmMSCs will remain while the other cells will be lysed.

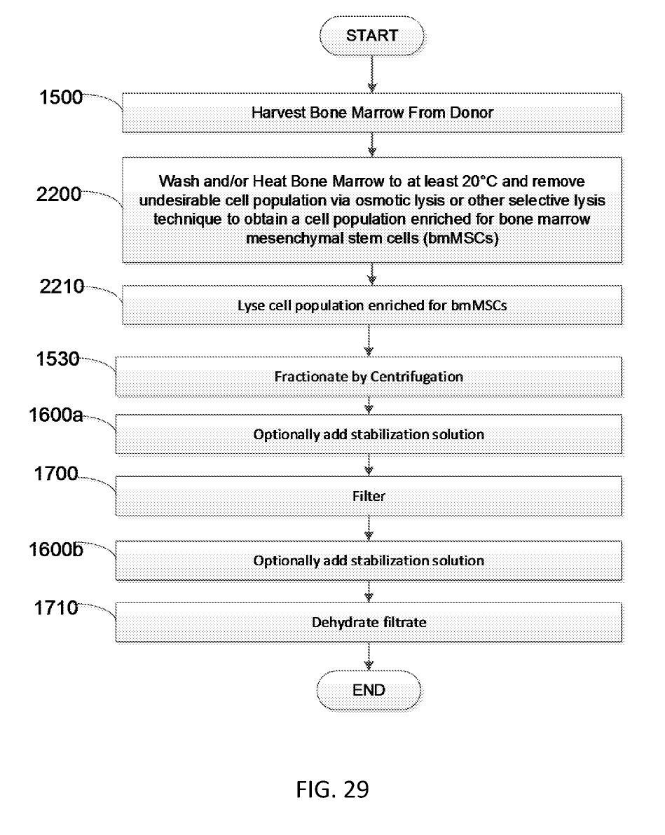

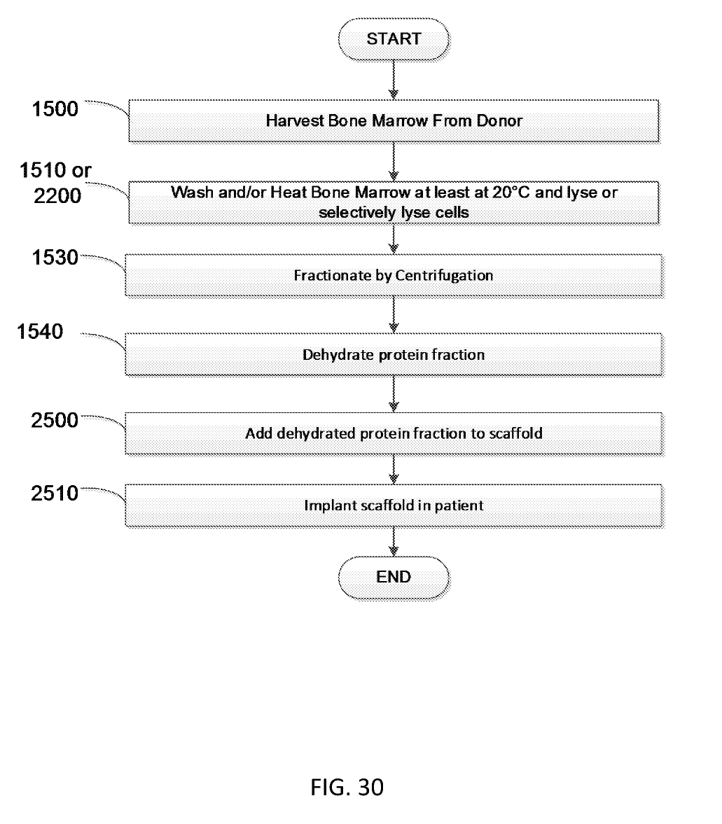

The bmMSCs or the cell population enriched for bmMSCs can be lysed 2210 to obtain bmMSC or primarily bmMSC derived proteins and/or other bioactive factors. As previously described, the lysate can be optionally fractionated by centrifugation 1530 and the desired proteins and/or bioactive factor containing fraction can be dehydrated 1540 as previously described. As shown in FIGS. 28 and 29, the method can include the optional steps of filtering 1700 after centrifugation 1530 and/or adding a stabilizer 1600 a,b after the step of centrifuging 1530 and/or filtering 1700.

It will be appreciated that other steps can be included in any of the methods described herein. In some embodiments, the method can include a pH altering step where an acid or a base or an acidic or basic solution can be added to product of any step in any method to result in a product that is acidic (pH less than 7), basic (pH greater than 7), or neutral (pH of 7). In some embodiments, after lysing, the lysate or product from any other subsequent step can be made more acidic, neutral, or basic as desired. In embodiments, the dehydrated product containing the soluble bone marrow derived proteins and/or bioactive factor(s) contains an acid that was introduced in the lysing step (e.g. 1510, 1910, or 2210). In other embodiments, the stabilization solution can contain an acid or base that can result in an acidic, basic, or neutral solution.

In some embodiments, the method can include a concentration step, where the product of any step in any embodiment of the method can be concentrated by a suitable technique. Suitable concentration techniques include but are not limited to, dehydration techniques (described elsewhere herein) and centrifugation based techniques. Other concentration techniques will be appreciated by those of skill in the art.

Methods of Making Scaffolds Containing a Soluble Bone Marrow Protein Composition

Methods of making the scaffold material, including VITOSS® material or CORTOSS® material, are described in U.S. Pat. Nos. 5,681,872; 5,914,356; 5,939,039; 6,325,987; 6,383,519; 6,521,246; 6,736,799; 6,800,245; 6,969,501; 6,991,803; 7,052,517; 7,189,263; 7,534,451; 8,303,967; 8,460,686; 8,647,614, which are incorporated by reference herein as if expressed in their entirety. Methods of making the dehydrated soluble bone marrow protein compositions are described herein. Methods and techniques of making or obtaining other suitable scaffold materials will be appreciated by those having ordinary skill in the art. In some embodiments, the scaffold material can be introduced during the production of making a soluble bone marrow composition where the scaffold material is mixed in at a step, such as the initial washing and/or lysing step with the initial starting bone marrow material.

Methods of Using the Soluble Bone Marrow Protein Compositions

The soluble bone marrow protein compositions (dehydrated or otherwise formulated as described herein) can contain an acid or be at an acidic pH. The soluble bone marrow protein compositions can be implanted into or otherwise administered to a subject in need thereof. In some embodiments, an effective amount of the soluble bone marrow derived protein composition (dehydrated or otherwise formulated) can be implanted or otherwise administered to a subject in need thereof. When implanted or administered, the proteins and/or other bioactive factors and the acid can be diluted and/or reconstituted by the bodily fluids of the subject. When this occurs, an acid microenvironment surrounding the proteins and/or other bioactive factors can be created. The acidic microenvironment surrounding the soluble bone marrow protein composition can facilitate solublization of the bone marrow derived proteins and/or other bioactive factors in the composition and can also facilitate the binding of the bone marrow proteins and/or other bioactive factors a scaffold (natural or synthetic), bone, cartilage, or other tissue of the subject at the site where the soluble bone marrow protein composition is deposited within the subject.

The soluble bone marrow protein compositions (dehydrated or otherwise formulated as described herein) can be added to a suitable scaffold or device. Suitable scaffolds include, but are not limited to, allogeneic, autologous, syngeneic, or xenogeneic complete extracellular matrix, decellularized extracellular matrix, or extracellular matrix components, hydrogels, synthetic or natural polymer solids and semi-solids, carbohydrates, self-assembling peptides, carbon nanotubes, collagen, calcium salts, chitosan, alginate, hyaluronic acid, bone powder, cartilage powder, proteins, sugars, plastics, metals, or combinations thereof. In some embodiments, the scaffold can be biocompatible. In other embodiments, the scaffold can be allogeneic, xenogenic, or autologous bone or demineralized bone. The scaffold can be flowable or non-flowable.

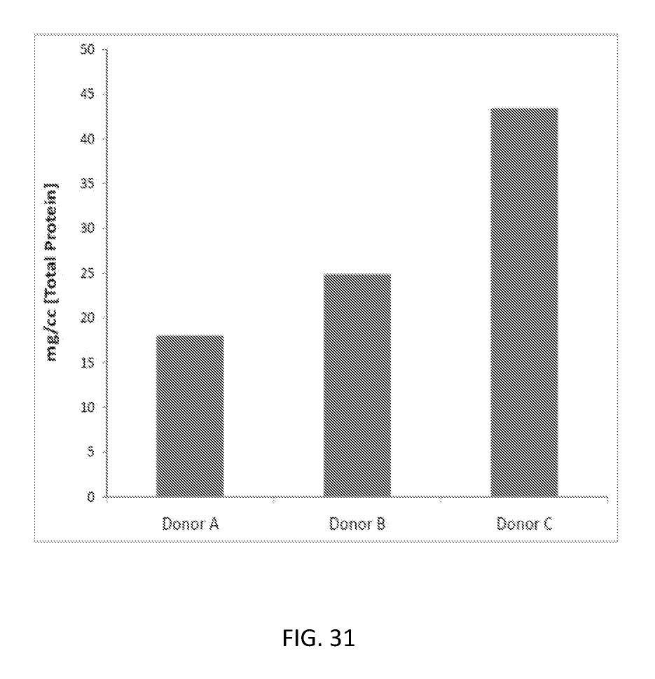

As shown in FIG. 30, the soluble bone marrow protein composition can be applied to a scaffold (implant) 2500, which is already present in a subject or can be implanted into a subject in need thereof 2510. When implanted 2510, the proteins in the dehydrated soluble bone marrow derived protein composition can solubilize and/or bind the scaffold when they come in contact a bodily fluid present in the subject. The acid present in the dehydrated soluble bone marrow derived protein composition can create an acidic microenvironment where the scaffold and/or soluble bone marrow protein composition is. The acidic microenvironment can facilitate solubilization of the bone marrow derived proteins and/or binding of the proteins and/or other bioactive factors to the scaffold (synthetic or natural) and/or other bone or tissue of the subject that are at the site of implantation. In some embodiments, the soluble bone marrow protein composition can be added in a dehydrated state to an implant material to encapsulate the proteins such as a putty, gel, or suspension.

In other embodiments, the soluble bone marrow derived protein composition can be applied directly into a scaffold already present in the subject in need thereof. As previously described, the proteins and/or other bioactive factors can be diluted or reconstituted when contacted with a bodily fluid present within the subject. As also described above, the acid that can be present in the bone marrow protein compositions described herein can create an acidic microenvironment that can facilitate solubilization and/or binding of the bone marrow proteins and/or bioactive factors to a scaffold present in the subject.

In some embodiments, the method can include the step of implanting or otherwise administering a soluble bone marrow protein composition or scaffold incorporating a soluble bone marrow protein composition as described herein to a subject in need thereof. In some embodiments, a method of treating a subject in need thereof can include the step of implanting or otherwise administering a soluble bone marrow protein composition or scaffold incorporating a soluble bone marrow protein composition as described herein to the subject in need thereof. In some embodiments, the subject in need thereof needs a bone graft or bone fusion. In some embodiments, the subject in need thereof has a bone and/or joint fracture or disease. In some embodiments, the subject in need thereof needs a spinal fusion. In some embodiments the compositions described herein can be used in patients with low bone density to prophylactically help reduce, delay, or prevent bone loss or fracture.

Methods of Using the Scaffolds Containing a Soluble Bone Marrow Protein Composition

The scaffold containing a soluble bone marrow protein composition as provided herein can be implanted in or otherwise administered to a subject in need thereof. The subject in need thereof can be in need of a bone grafting or bone fusion procedure. As such, in some embodiments a method can include the step of implanting or administering an implant containing the scaffold material described herein (including those scaffolds containing a soluble bone marrow protein composition) to a subject in need thereof. In some embodiments, the subject in need thereof can be in need of a bone graft or a bone fusion. In some embodiments, a method of treating a subject in need thereof can include the step of implanting or administering an implant containing a scaffold (including those scaffolds containing a soluble bone marrow protein composition) described herein to a subject in need thereof. In some embodiments, the subject in need thereof is in need of a bone graft or a bone fusion. In some embodiments, the subject in need thereof has a bone fracture, diseased bone, joint fracture, or diseased joint.

Spinal Fusion and Grafting.

Many patients affected by severe back pain due to degeneration of one or more discs are often treated with spinal surgical procedures. It is estimated that each year at least 500,000 spinal fusion procedures are performed in the United States. In cases where the patient has advanced disc degeneration or spinal instability, a fusion procedure can involve a surgical incision in the patient's back or abdomen to access and remove the affected disc material. To provide initial stability and support of the surrounding vertebrae, the resulting defect can be filled with a structural implant made of either titanium, shaped bone derived from a human cadaver, or a synthetic material known as polyetheretherketone (“PEEK”). Adjunctively, these procedures can require the use of bone grafting material to repair defects and facilitate the fusion of two bony elements. A scaffold, such as VITOSS® material, containing the soluble bone marrow protein composition can provide an alternative to patient- or cadaver-derived tissues in spinal fusion and/or grafting procedures. In some embodiments, a method of fusing a portion of the spine, where the method includes the step of implanting or administering an implant containing the scaffold described herein to a subject in need thereof.

Trauma.

Physical trauma such as falls and accidents can result in bone fracture or damage. Fractures of broken bones are often realigned with hardware, such as plates, rods and screws. Once the hardware has been used to recreate the skeletal anatomy and to provide the stability of the bony structure, there are often defects or voids in the bone which remain. Those voids may require the use of bone graft material. The goal of bone grafting in trauma applications is to rapidly heal the damaged bone. Approximately 250,000 trauma related bone graft repairs are performed annually on a worldwide basis. The scaffold, such as VITOSS® material, containing the soluble bone marrow protein composition can be used as a bone graft substitute in a variety of trauma applications, including those of the extremities, spine and pelvis.

For patients with poor bone healing, as seen in osteoporotic patients, CORTOSS containing the soluble bone marrow protein composition can be used in a variety of surgical procedures to quickly provide structural stability and reinforcement of the bones after surgery. The surgeon's goal is to repair the patient's bone and enhance the patient's mobility as quickly as possible since prolonged bed rest or inactivity may result in decreased overall health for older or osteoporotic patients. A scaffold, such as CORTOSS® material, containing the soluble bone marrow protein composition can be made as a simple mix-on-demand delivery system that can allow for minimum waste and maximum ease of use and flexibility for the surgeon. The scaffold, such as CORTOSS® material, containing the NR soluble bone marrow protein composition can be configured as an injectable material that is delivered to a subject through a pre-filled, unit dose, disposable cartridge.

In some embodiments, a method of fusing a portion of the spine, where the method includes the step of implanting or administering an implant containing a scaffold (including scaffolds containing a soluble bone marrow protein composition) described herein to a subject in need thereof. In some embodiments, a method of bone grafting, where the method includes the step of implanting or administering an implant containing a scaffold (including scaffolds containing a soluble bone marrow protein composition) described herein to a subject in need thereof.

Bioactive Factors and/or Biocompatible Materials

Various embodiments of the present disclosure relate to bioactive factors and/or biocompatible materials that stimulate tissue growth. As can be appreciated these bioactive factors can be derived from soft tissue and/or physiological solutions containing cells. Physiological solutions may exist as solutions naturally in the body or be derived from tissue when the cells are extracted. Any tissue containing cells may be a source of physiological fluid, such as, for example, mesodermal, endodermal, and ectodermal tissues. Examples of these tissues include bone marrow, blood, adipose, skin, muscle, vasculature, cartilage, ligament, tendon, fascia, pericardium, nerve, and hair. These tissues may also include organs such as the pancreas, heart, kidney, liver, intestine, stomach, and bone. The cells may be concentrated prior to processing as described by the current disclosure. In certain aspects, as used herein soft tissue can be any tissue containing cells may be a source of physiological fluid, such as, for example, mesodermal, endodermal, and ectodermal tissues. Examples of these tissues include bone marrow, blood, adipose, skin, muscle, vasculature, cartilage, ligament, tendon, fascia, pericardium, nerve, and hair. In certain aspects, bone, cancellous bone especially, is not a soft tissue and a tissue harvested for use with osmolarity agents intended to produce osmotic shock.

In accordance with one embodiment, a portion of cancellous, corticocancellos and/or cortical bone or any combination thereof can be harvested from a donor. In one embodiment, the harvested material can be harvested in such a way as to retain as much bone marrow in the harvested sample as possible.

The harvested sample can be exposed to lysing conditions and/or a lysing agent to facilitate lysis of the cells therein to release growth factors and nutrients contained sample. In other words, the harvested sample can be exposed to a lysing agent that lyses the cells within the harvested sample. Once cellular components are lysed, they release growth factors and/or bioactive materials, such as cytokines and nutrients, to stimulate growth, differentiation, and repair. These growth agents can be cytokines such as proteins, hormones, or glycoproteins including members of the TGF-β family (including bone morphogenetic proteins), interleukins, interferons, lymphokines, chemokines, platelet derived growth factors, VEGF, and other stimulative agents that promote growth, repair or regenerate tissues.

In other embodiments, cells from other tissues can be lysed to release growth agents that can be binded to the harvested sample and further processed as an implant. Lysing conditions may be mechanical in nature such as thermolysis, microfluidics, ultrasonics, electric shock, milling, beadbeating, homogenization, french press, impingement, excessive shear, pressure, vacuum forces, and combinations thereof. Excessive shear may be induced by aggressive pipetting through a small aperture, centrifuging at excessive revolutions per minute resulting in high gravity forces. Rapid changes in temperature, pressure, or flow may also be used to lyse cellular components. Lysing conditions can include thermolysis techniques that may involve freezing, freeze-thaw cycles, and heating to disrupt cell walls. Lysing conditions can also include microfluidic techniques that may involve osmotic shock techniques of cytolysis or crenation. In certain embodiments as described herein, embodiments that involve osmotic shock do not involve cancellous bone.

Lysing conditions can also include the imposition of ultrasonic techniques, including, but not limited to, sonication, sonoporation, sonochemistry, sonoluminescence, and sonic cavitation. Lysing conditions can also include electric shock techniques such as electroporation and exposure to high voltage and/or amperage sources. Lysing conditions can further include milling or beat beating techniques that physically collide or grind cells in order to break the cell membranes, releasing the stimulative agents contained within.

Lysing can also be accomplished by exposing cells of the harvested sample to a lysing agent, which can facilitate release of stimulative growth agents include lysis due to pH imbalance, exposure to detergents, enzymes, viruses, solvents, surfactants, hemolysins, and combinations thereof. Chemical induced lysis of the cells by pH imbalance may involve exposure of cells of the harvested sample to a lysing agent in order to disrupt the cell walls and release soluble growth agents. In some embodiments, a lysing agent can include one or more acids and/or bases.

After exposure to the lysing agent, the harvested sample may be exposed to buffers or other solutions to substantially neutralize the pH of the mixture of the growth factors and the lysing agent. In some embodiments, it may be desired that the pH be acidic (e.g., pH below 7) or basic (e.g., pH above 7) to retain solubility of particular growth factors or bioactive agents. For example, bone morphogenetic proteins (particularly BMP-2, BMP-4, BMP-6, BMP-7, BMP-9, BMP-14, and other bone morphogenetic proteins 1-30) are more soluble at acid pH values under 7 than neutral or basic pH.

In other embodiments, a lysing agent can include a volatile acid or base, such as acetic acid or ammonia, and the cellular material, after exposure to the lysing agent, may be neutralized or partially neutralized by drying techniques such as evaporation, vacuum drying, lyophilization, freeze drying, sublimation, precipitation, and similar processes as can be appreciated. In yet other embodiments, a lysing agent can include detergents that can disrupt cell walls and remove any lipid barriers that may surround the cell. Enzymes, viruses, solvents, surfactants, and hemolysins can also help cleave or remove outer cell membranes releasing the bioactive growth agents contained within.

The use of these lysing agents and/or exposure of the harvested sample to lysing conditions may be followed by neutralization, as noted above, and/or another secondary process to remove any undesired remnants. The growth agents, nutrients, etc., released by the lysing process may be added to a carrier such as a synthetic scaffold, non-bone biologic scaffold (e.g. collagen or other non-bone tissue scaffold). In yet other embodiment, a harvested non-bone sample, acting as a carrier can be exposed to lysing conditions and/or a lysing agent, and bioactive factors released by the lysing process can be binded to at least a portion of the sample. In some embodiments, the growth agents released by lysing of cellular material may be used immediately for autologous use. In other embodiments, the released growth agents may be stored for allogenic use (e.g. separately from the tissue they were derived from) Storage techniques can include freezing or lyophilization to preserve bioactivity. The growth factors and nutrients may also be frozen or lyophilized on the chosen carrier to allow for complete binding of the stimulative agent to the carrier and to allow for immediate use by the surgeon. Lyophilization also allows for greater room temperature shelf life and an opportunity for concentration into a smaller volume.

Another embodiment of the present disclosure relates to obtaining a specific set of growth factors and nutrients from a physiological solution containing cells. In this embodiment, cells are lysed as described above and the lysate solution is subjected to materials with a charged surface, including, but not limited to, chromatography resins, ceramics, soft tissues, and other materials with an electric charge. The charged surface attracts certain stimulative growth agents and molecules removing them from the lysate solution. The remaining growth agents can then be used to regenerate or repair the desired tissue type. Similar to the previous embodiment, the growth agent solution can be further concentrated and frozen or lyophilized in order to extend shelf life.

Another embodiment of the disclosure includes selectively rinsing, lysing, or removal of certain cellular components while retaining other cellular components. Selective lysing or removal can be accomplished physically by methods described above. As can be appreciated, certain cells can be resistant to various lysing mechanisms. As a non-limiting example, mesenchymal stem cells (MSC) are resistant to cytolysis and osmotic lysis due to their resistant cell walls and ineffective cells volumes. Accordingly, to accomplish selective lysing, osmotic lysis can be used to lyse red and white blood cells from blood or bone marrow. Once the non-resistant cells are lysed, the resulting solution is an enriched MSC population. The solution can then be further concentrated via centrifugation, florescence-activated cell sorting (FACS), filtration, magnetic bead selection and depletion, and/or gravity sedimentation. For allogeneic transplantation, FACS and magnetic bead separation and depletion can be useful in removing any remaining cells that would cause an immune response from the implant patient. Once implanted, cells can function in a homologous manner and differentiate in the desired phenotype.

Another embodiment of the disclosure includes a combination of previous two embodiments. A physiological solution may be enriched by selective lysis and further concentrated by centrifugation, FACS, magnetic bead selection and depletion, and/or gravity sedimentation. The enriched physiological solution is added to a physiological solution that has been lysed in the methods described previously in order to help induce differentiation of the cells into the desired phenotype. These cells can then function in the desired manner to regenerate and repair tissues.

In another embodiment, cancellous bone may be exposed to a weak lysing agent (such as less than 1M acetic acid) that only partially lyses the cell population present. In this embodiment, the partial lysis releases growth factors and binds them to the bone while other cells, such as mesenchymal stem cells and progenitor cells, may still remain viable and attached to the bone.

In another embodiment, cancellous bone may be exposed a weak lysing agent (such as water) and then subjected to mechanical lysing conditions previously stated (such as thermolysis, high/low pressure, sonication, centrifugation, etc.). Once the cells have lysed, the bone, cell fragments, and debris are removed from the solution containing the growth factors. The solution may then become positively charged by the addition of an acid or another proton donor fluid. The growth factors in the solution may then be further concentrated using techniques described, frozen, or lyophilized into a soluble powder. The soluble powder could be reconstituted with a fluid prior adding it to an implant during surgery or added in the dry powder form to an implant prior to implantation.

In another embodiment, a bioactive factor (e.g. a growth factor) can be formed from non-bone physiological fluids containing cells. The cells can be lysed as described elsewhere herein. The bioactive factors released can be retained and stored and/or loaded onto a carrier.

In another embodiment, a physiological fluid containing cells, such as synovial fluid, may be harvested from a live donor, cadaveric donor, or autologously. The fluid may be subjected to mechanical or chemical lysing conditions described in order to solubilize growth factors. Once the growth factors are released from the cells, the solid materials (such as cells fragments, debris, or platelets) may be removed by processes described such as filtration, centrifugation, or gravity sedimentation. Once the solid materials are removed, the solution may be then become positively charged by the addition of an acid or another proton donor fluid. The growth factors in the solution may then be further concentrated using techniques described, frozen, or lyophilized into a soluble powder. The soluble powder could be reconstituted with a fluid prior adding it to an implant during surgery or added in the dry powder form to an implant prior to implantation. Alternatively, cartilage with or without synovial fluid can be prepared in a similar fashion for the repair and regeneration of cartilage or spinal discs. In addition, other tissues such as muscle, adipose, nerve, dermis, cardiac tissue, vascular tissue, nucleus pulposus tissue, annulus fibrosus tissue, or other solid tissues can be prepared in this fashion to be used to help repair or regenerate tissues.

Stimulative growth agents can be derived from various cellular solutions. These solutions may comprise cultured and/or uncultured cells, and can be autologous, allogeneic, or xenogeneic in origin. If the cells are allogeneic or xenogeneic in origin, at least partial lysing or immune cells depletion by methods previously described can be performed so that the stimulative growth agents do not elicit an immune response in the patient. Alternatively, immune response agents, such as CD45+ cells and other leukocytes, may be removed prior to use to reduce or eliminate immune response. These immune response agents may be removed by the selective lysing as previously described in this disclosure.

Various embodiments of the present disclosure relate to compositions and/or methods for providing an anti-microbial polysaccharide scaffold that may be combined with an osteostimulative agent such as bioactive growth factors and different types of cells to stimulate tissue growth, cell adhesion, cell proliferation, and enhanced wound healing. Chitosan is a polysaccharide found in marine crustacean shells and the cell walls of bacteria and fungi. Chitosan is a non-toxic biocompatible material that can support tissue growth. With the combination of biocompatibility, antibacterial activity, versatility in processing, and ability to bind cells and growth factors, chitosan is a distinguished biomaterial to support in tissue growth. The materials including viable cells may be customized for use within the applications such as, but not limited to; void fillers and implants for tissues or bone. hemostatic agent, wound covering, osteoncology, and treatment of infected site. The scaffold may also include minerals.

In one embodiment, a biocompatible shape memory osteoconductive and/or osteoinductive anti-microbial compressible implant scaffold may be used in tissue engineering. For example, the present disclosure provides an orthopedic structure comprising a chitosan solution and a non-toxic mineral mixture resulting in a compressible solid porous substrate.

The scaffold may comprise chitosan with a weight percentage in the range of about 5% to about 80%, in the range of about 10% to about 70%, and/or in the range of about 15% to about 60%. In some embodiments, the chitosan concentration is greater than about 5%, greater than about 30%, or more. In other embodiments, the chitosan concentration is less that about 10% or less than about 2.5%.

In accordance with various implementations of the present disclosure, the chitosan molecular weight may be in a range of between about 1 kDa and about 750 kDal, in a range of between about 10 kDal and about 650 kDa, and/or in a range of between about 50 kDa and about 550 kDa.

The chitosan used may be deacetylated chitosan. According to one implementation, the degree of deacetylation may range from, but is not limited to, about 50% to about 99% deacetylation. Generally, the lower the percentage/degree of deacetylation, the more rapid the degradation takes place when implanted. The deacetylation percentages may also be tailored to specific tensional and compressive properties. The lower the deacetylation the lower the tensile strength of the scaffold.

In accordance with various implementations, the deacetylation percentage of the chitosan can be in a range from about 50% to about 66.6% in order to produce more rapid degradation profile and in turn have a lower density affecting porosity. In other implementations, the deacetylation percentage of the chitosan can be in a medium range from about 66.6% to about 83.2% in order to produce a medium degradation profile and in turn have a medium density affecting porosity. In accordance with yet other implementations, the deacetylation percentage of the chitosan can be in a medium range from about 83.2% to about 99% in order to produce a longer degradation profile and in turn have a higher density affecting porosity.

The chitosan material may be compounded with an additional protein or amino acid to improve protein and cell binding. Examples of proteins, enzymes, structural proteins, cell signaling or ligand binding proteins, or amino acids include, but are not limited to, collagen, glutamic acid, and lysine. The chitosan may be medical grade or may be of equivalent quality containing low level of toxic contaminants such as heavy metals, endotoxins and other potentially toxic residuals or contaminants.

In accordance with various embodiments of the present disclosure, the chitosan solution can be prepared by dissolution in low pH fluids, such as acids. Low pH fluids include, but are not limited to, acetic, hydrochloric, phosphoric, sulfuric, nitric, glycolic, carboxylic, or amino acids. The amount of acid used may be between about 0.1% to about 50%, and/or may be between about 0.1% and about 25%. In some embodiments, the pH can range from slightly acidic to neutral or partially neutral. Neutralization can be obtained by using base substances such as, but not limited to, sodium hydroxide, ammonia hydroxide, potassium hydroxide, barium hydroxide, caesium hydroxide, strontium hydroxide, calcium hydroxide, lithium hydroxide, rubidium hydroxide, butyl lithium, lithium diisoprpylmadie, lithium diethylamide, sodium amide, sodium hydride, and lithium bis(trimethylsily)amide. Neutralization may also be obtained by using basic amino acids including lysine, histidine, methyllysine, arginine, argininosuccinic acid, L-arginine L-pyroglutamate, and ornithine. Different techniques to achieve neutralization may be used such as evaporation, vacuum drying, lyophilization, freeze drying, sublimation, precipitation, and similar process as can be appreciated. The resulting solution results in a suspension or gel comprising chitosan with a liquid medium being at least partially comprised of water. The suspension or gel may also include mineral particles.

The resultant chitosan/mineral suspension may then be shaped to desired forms such as porous solids or semisolids through techniques such as injection molding, vacuum molding, injection compression molding, rotational molding, electrospinning, 3D printing, casting, and phase separation. The shapes may be orthopedic shapes such as, for example, dowels, tubes, pins, screws, plates, wedges anchors, strips, bands, hooks, clamps, washers, wires, fibers, rings, sheets, spheres, and cubes.

In accordance with another aspect of the disclosure, the chitosan scaffold may have a matrix porosity ranging from about 1 μm to about 5 mm. The matrix scaffold may also have a different surface porosity compared to its internal porosity. The surface porosity may have ranges from about 1 μm to about 1 mm, while the internal porosity may range from about 10 μm to about 5 mm. Overall pore size can be dependent on concentrations of chitosan, lower concentrations will result in larger pore size while higher concentration will result in smaller pore size. Pores size may also be designed to align vertically, longitudinally, horizontally, or a combination thereof depending on the process used during preparation or the intended site of implantation. Size and direction of the pores and channels may be designed and controlled through control rate freezing, and directional freezing. Variables such as freezing rate, freezing temperature, and specified area of freezing can be changed to adjust pore/channel size and direction due to the functions of the temperature gradient. An implant can be frozen at a ramp down rate of −0.1° C. to −15° C. every 1 minute to 20 minutes, creating uniform crystal formation. After freeze drying, the crystals evaporate leaving pores within the implant. For example, a slow ramp down rate of −10° C. every 10 minutes will result in larger pore size, while a fast ramp down rate of −10° C. every 1 minute results in smaller pore size. Channels instead of pores can be formed by decreasing the ramp down rate even further to −5° C. every 15 minutes. Pore and/or channel directionality can designed by applying the freezing source during freeze drying to a specified area of the implant. For example, if the freezing source is applied to a specified area (e.g., a specific surface) of the implant, the pore or channel direction will be perpendicular to the freezing source. A combination of applied freezing sources can result in multidirectional pore or channel structure. If the freezing source is not placed in any specified area, then the pore or channel direction can be anisotropic.

In accordance with another aspect of the present disclosure, the implant may have shape memory once hydrated with liquid. A dehydrated or hydrated sponge may be compressed circumferentially, unilaterally, or in multiple directions up to about 10 times its original size but when hydrated goes back to its original shape. The scaffold can be compressed into various shapes such as, but not limited to, tubes, pins, cubes, strips, and sheets. Compression may occur externally directed towards the scaffold or internally directed outward from the scaffold.

In some embodiments, the biocompatible implant may include minerals such as calcium salts (e.g., calcium phosphate), silicate, carbonate, sulfate, halide, oxide sulfide phosphate, metals or semimetals including gold silver copper, alloys, and/or a combination thereof. In accordance with one aspect of the present embodiment, calcium phosphate may be selected from hydroxyapatite (HA), silicate hydroxyapatite (HA), tri-calcium phosphate (TCP), pure/substituted beta tri-calcium phosphate (β-TCP), alpha tri-calcium phosphate (α-TCP), octalcalcium phosphate (OCP), tetralcalcium phosphate (TTCP), dicalcium phosphate dehydrate (DCPD), and/or a combination thereof. Mineral particle sizes may range from a powder of about 1 nm to about 1 mm. The mineral content can also be added in a granule size ranging from about 50 μm up to about 5 millimeters. The implant may include granules larger than 100 μm to increase compression resistance and cell/protein binding. The calcium salt concentration may be greater than about 10%, greater than about 30%, or greater than about 40%.

The scaffold may comprise a mineral in a range of about 5% to about 75%, in a range of about 8% to about 72%, and/or in a range of about 10% to about 70%.

In accordance with yet another aspect of the disclosure, the implant contains antimicrobial and/or antibacterial properties which are dependent on the amount of chitosan and pH levels that are used in the formulation. The chitosan concentration along with the pH can provide antimicrobial activity against but not limited to the following organisms; staphlyococcus aureus (MRSA), Enterococcus faecalis (VRE), Acinetobacter baumanii, Escherichia coli, Klebsiella pneumoniae, Streptococcus pyogenes, Staphylococcus epidermidis, Alomonella choleraesuis, Pseudomonas aeruginos, Enterococcus faecalis, Serratia marcescens, Stenotrphomonas maltophilia, Streptococcus mutans, Clostrium difficle, Streptococcus pneumoniae, shigella species, Enterobacter aerogenes, Proteus mirabilis, Proteus vulgaris, Citrobacter freundii, Enterobacter cloacae, Moraxella catarrhalis, Micrococcus luteus, and Vibrio cholera. The material also increases in stiffness after an increase in pH. In some embodiments, the chitosan solution can range from about 5 mg/mL to about 200 mg/mL. The pH level may be less than 8 and/or less than 7.

In accordance with various embodiments, the scaffold tensile, torsional, shear, and compressive properties can be strengthened by crosslinking using methods such as, dehydrothermal, chemical, physical, or photometric crosslinking. Dehydrothermal crosslinking may involve exposing the scaffold to elevated temperatures with or without the use of negative pressure. Chemical crosslinking may include treatment with nitrous acid, malondiadehyde, psoralens, aldehydes, formaldehydes, gluteraldehydes, acetalaldehyde, propionaldehyde, butyraldehyde, bensaldehyde, cinnamaldehyde, and/or tolualdehyde. Photometric crosslinking may use energy and/or light sources that may include ultraviolet, plasma, or other energy sources.

In various embodiments, a biocompatible osteoconductive and/or osteoinductive anti-microbial implant scaffold may be used use in tissue engineering. An orthopedic structure comprising a chitosan solution includes one or more substances including growth factors, growth factor stimulative agents, vitamins, and/or biologically active molecules. Calcium salts (e.g., calcium phosphate) may also be included as an osteostimulative agent.

Growth factors in the materials having viable cells can include, but are not limited to, bone morphogenetic protein (BMP), transforming growth factor β (TGF-β), growth differentiation factor (GDF), cartilage derived morphogenetic protein (CDMP), interlukins, interferon, lymphokines, chemokines, platelet derived growth factors (PDGF), VEGF, β-fibroblast growth factor (β-FGF), fibroblast growth factors (FGF), and other stimulative agents that promote growth, repair or regenerate tissue. Bone morphogenetic protein may be selected from BMP-2, BMP-3, BMP-4, BMP-5, BMP-6, BMP-7, BMP-8, BMP-9, BMP-10, BMP-11, BMP-12, BMP-13, BMP-14, BMP-15, and BMP-16. The bone morphogenetic protein may also be recombinant human bone morphogenetic protein. Growth factors may also be angiogenic or mitogenic growth factors.

In another embodiment, a biocompatible osteoconductive and/or osteoinductive anti-microbial implant scaffold may be used in tissue engineering. An orthopedic structure comprising a chitosan solution and a mineral mixture includes seeded cells. The cells can comprise of mesechymal stems cell (MSC), adipocytes, chondrocytes, osteocytes, fibroblasts, osteoblasts, preosteoblasts, osteprogenitor cells, and combinations thereof.

In various embodiments, a biocompatible osteoconductive and/or osteoinductive anti-microbial malleable implant scaffold may be used in tissue engineering. An orthopedic structure comprising a chitosan solution and a mineral mixture has a putty-like consistency. The material may be molded to meet different situations. Formulation parameters may be adjusted to have different viscosities and adhesion characteristics based on the application.

In alternative embodiments, a biocompatible osteoconductive and/or osteoinductive anti-microbial flowable implant scaffold may be used in tissue engineering. An orthopedic structure comprising a chitosan solution and a mineral mixture has a flowable consistency. The material may be tailored to meet different situations. Viscosity parameters may be formulated to have less viscous properties in applications such as pastes, injectable gels and sprays. The paste and gels can be applied into the body in the desired shape, to aid in the efficacy of the application. A less viscous formulation such as putty or a very viscous injectable/flowable fluid can be applied in places such as bone voids, bioinert implants, cannulated screws, around screws, or other orthopaedic applications.

In various other embodiments, a biocompatible osteoconductive and/or osteoinductive anti-microbial coating implant scaffold may be used in tissue engineering. An orthopedic structure comprising a chitosan solution and a mineral mixture has a low viscosity consistency for coating purposes. The coating may be applied to bioinert materials such as, but not limited to, peek, stainless steel, titanium, radel, and silicone structures. For example, a coating can be applied to (e.g., sprayed on) bioinert implants such as, but not limited to, cages, screws, screw heads, pins, rods, wires, dowels, connectors, hip stems, acetabular cups, and plates. A coating may also be applied to (e.g., sprayed on) bioactive implants such as, but not limited to, minerals, autograft, allograft, xenograft, and collagen.

The systems and methods described herein can be employed in surgical environments where the implantation of stimulative growth agents in a patient is desired. Although the present disclosure describes the methods and systems for producing stimulative growth agents, particularly ones derived from physiological fluids containing cells or cellular tissues, it is understood that the methods and systems can be applied for a wide variety of medical applications including ones directed at regeneration or repair of bone, cartilage, muscle, tendon, ligament, vasculature, fat, annulus fibrosus, nucleus pulposus, skin, hair, blood, lymph nodes, fascia, neural, cardiac, pancreatic, hepatic, ocular, dental, digestive, respiratory, reproductive, and other soft tissue applications, such as in regenerative medicine and tissue engineering.

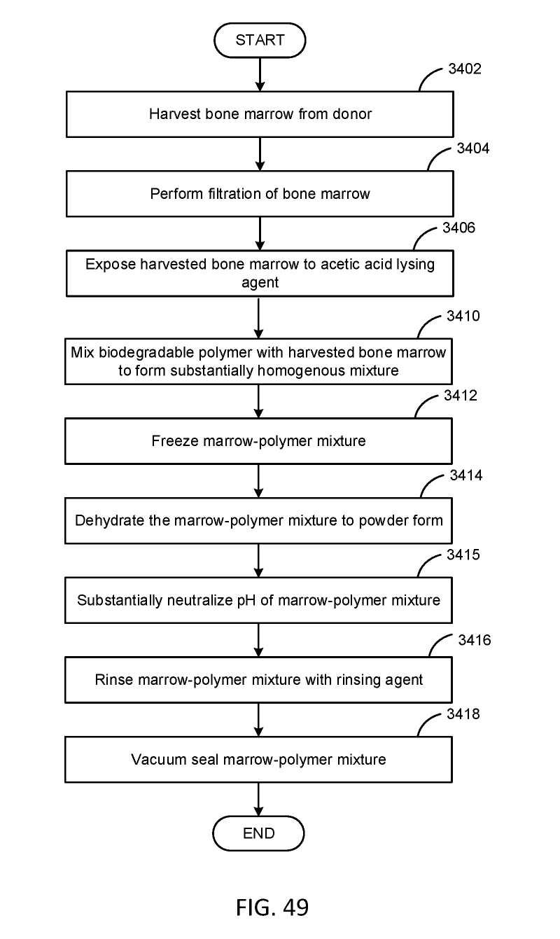

Reference is now made to FIG. 45, which depicts a method in accordance with one embodiment of the disclosure. In the embodiment illustrated in FIG. 45, an implant that can be suitable for bone applications is shown. In the embodiment of FIG. 45, cancellous bone is recovered from a cadaver, live donor, or harvested autologously from a patient in box 3002. The harvested cancellous bone can be ground or cut to a desired shape and configuration as can be appreciated. Care may be taken to retain some cellular material, bone marrow, and/or blood within the bone during harvest and cutting operations. In prior art implants, bone marrow and/or blood within the bone can be systematically removed and/or cleaned from the harvested bone sample. In an embodiment of the disclosure, cancellous bone may have cortical bone portions such as in the iliac crest, vertebral bodies, chondyles, etc.

The cancellous bone is then exposed to acetic acid in box 3004, which acts as a lysing agent as described above. In one embodiment, the acetic acid concentration can be greater than 1%, in a molarity range of 0.2M-17M. The acetic acid lysing agent is employed to lyse cells remaining in the porous bone structure and on bone surface of the cancellous bone. The lysing of the cells releases and solubilizes growth factors and bioactive materials contained in the cellular material. Additionally, pH of the harvested bone may be substantially neutralized in box 3008. In some embodiments, the pH of the harvested bone can be neutralized by the rinsing agent and rinsing step in box 3006. In other embodiments, pH neutralization may not be required. Further pH neutralization of the harvested bone may be accomplished by dehydrating in box 3010 by evaporation, vacuum drying, or lyophilization to reduce the acetic acid lysing agent to a residue and bring the implant to a more neutral pH.

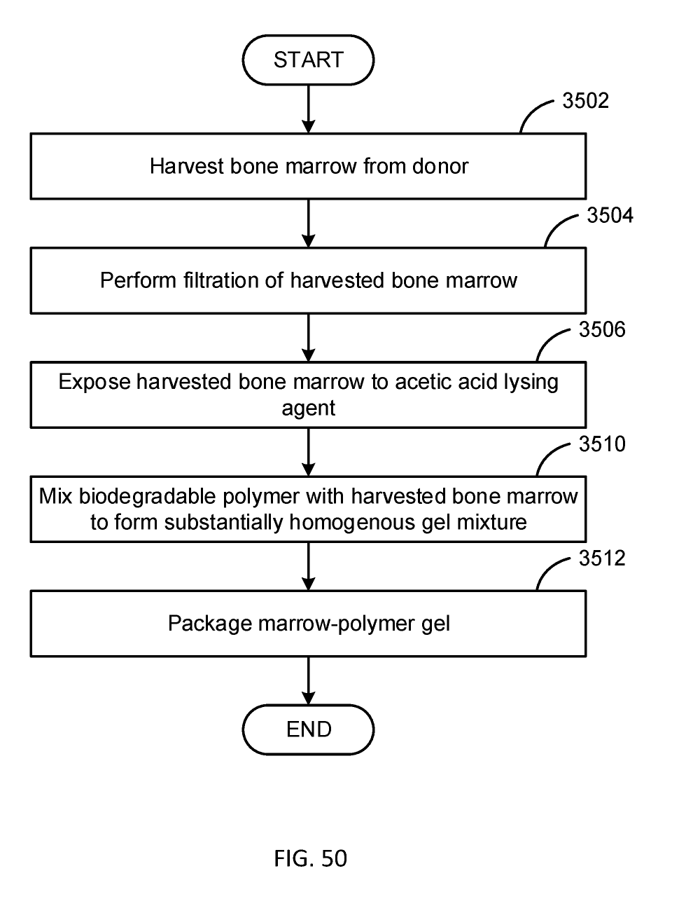

Rinsing solutions can be water, saline (NaCl, PBS, etc.), peroxides, alcohol (isopropyl, ethanol, etc.), crystalloids, sterilizing fluids (antibiotics such as gentamicin, vancomycin, bacitracin, polymixin, amphotericin, ampicillin, amikacin, teicoplanin, etc.), preserving fluids (DMEM, DMSO, mannitol, sucrose, glucose, etc.), storage agents, and/or other fluids used in processing of allografts. Reference is now made to FIG. 46, which depicts an alternative embodiment of the disclosure. Bone marrow is harvested from a cadaver, live donor, or harvested autologously from a patient in box 3102. If a cadaver donor is used, a higher volume of marrow may be obtained by harvesting the marrow before any bone sectioning is done. In some embodiments, using a cannulated drill attached to a vacuum line to harvest marrow would also increase the yield of bone marrow from a cadaver donor. The tip of the cannulated drill breaks apart within the cancellous bone, allowing the vacuum to pull marrow through the cannula into a collection chamber.

Harvesting marrow from a living donor prior to the donor being removed from life support can also be employed as a marrow harvesting technique, because as the marrow is removed, blood flow caused by physiological circulation flushes additional bone marrow material into the area for further aspiration. After marrow has been harvested, particular cell types (such as mesenchymal stem cells, osteoblasts, osteocytes, or other progenitor cells) may be concentrated by filtration, centrifugation, magnetic bead binding, fluorescence activated cell sorting (FACS), and/or other cell sorting or concentration techniques as can be appreciated to increase the cell concentration, fractionate cell types, or eliminate particular cell types from the solution in box 3104. Once, the desired cell population is obtained, it may be exposed to a lysis technique previously described, such as exposure to acetic acid in box 3106.

Once acetic acid is added to the cells, they are given time to lyse and the growth factors and other bioactives are solubilized. The solution can be centrifuged or filtered to eliminated any cell fragments or cellular debris. The solution may undergo a second filtration step to remove other solid precipitates such as precipitated hemoglobin. The solution may undergo a third filtration step to concentrate the growth factors and other bioactives in the solution. The solution is then dehydrated by methods previously described, such as lyophilization. The solution is reduced to a water soluble powder in box 3110 and may be sealed under vacuum to increase shelf-life in box 3112. The solution can also be frozen to increase shelf life. This powder can be rich in a number or bioactive molecules and/or growth factors including, but not limited to, BMP-2, VEGF, αFGF, FGF-6, TGF-B1, and others as can be appreciated.

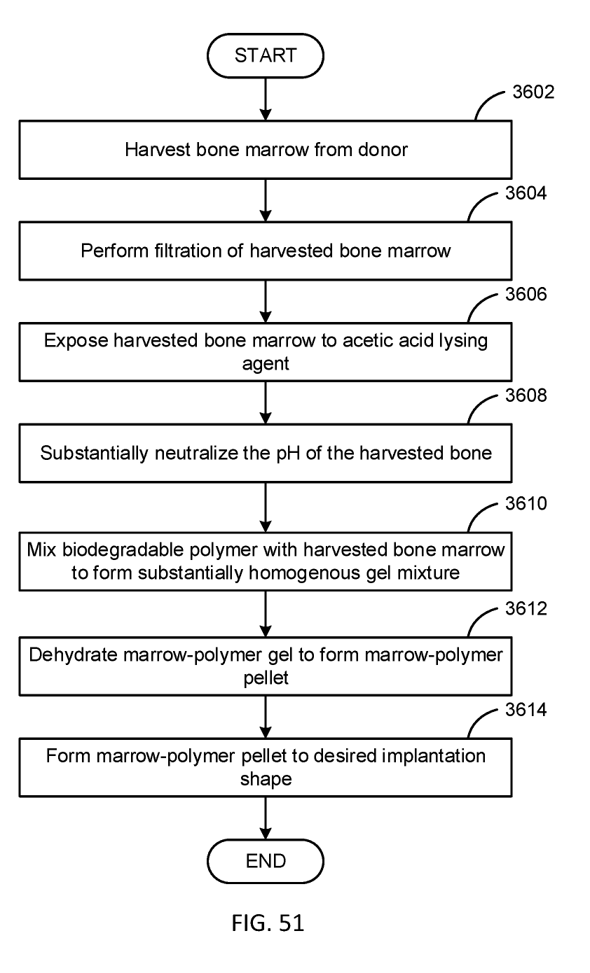

Reference is now made to FIG. 47, which depicts an alternative embodiment of the disclosure. In the depicted embodiment, cancellous bone is recovered from a cadaver, live donor, or harvested autologously from a patient in box 3202. If required by a particular implant application, the harvested cancellous bone may be ground or cut to a desired shape and configuration. Care may be taken to retain as much bone marrow and blood within the bone during harvest and cutting operations. Cancellous bone may have cortical bone portions such as in the iliac crest, vertebral bodies, chondyles, etc. Accordingly, the cancellous bone may have cortical portions removed prior to further processing. The harvested cancellous bone is then exposed to a lysing agent, such as water, to lyse the cells contained in the cancellous bone in box 3204. If a particular anticoagulant, such as heparin, is used as a lysing agent, the growth factors released by lysing the cells will be solubilized in solution. If no anticoagulant is used or if a different anticoagulant is used, such as sodium citrate, the cells will be lysed and release growth factors, but they will not be fully solubilized in the fluid.

In this case, the bone is then removed from the fluid in box 3206 and a solubilization agent, such as an acid, is added to the fluid to solubilize the growth factors and other bioactives in box 3208. Once the growth factors and other bioactives have been solubilized, the fluid may be neutralized and/or lyophilized in box 3210. If acetic acid was used as the solubilizer, neutralization may be unnecessary as a substantial amount of acetic acid will vaporize during lyophilization. Alternatively, other lysing agents and solubilizers could be used to lyse the cells and solubilize the growth factors, preventing the growth factors and bioactive materials from binding to the cancellous bone from which the cells were harvested.

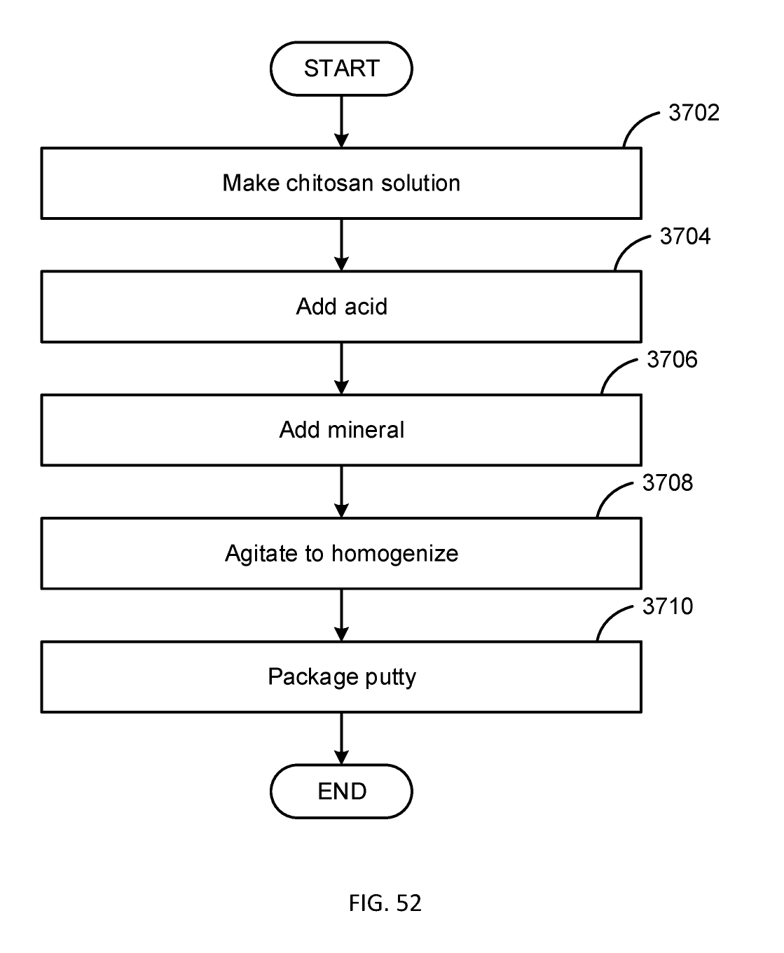

Reference is now made to FIG. 48, which depicts an alternative embodiment of the disclosure. In the depicted embodiment, soft tissue is recovered from a cadaver, live donor, or harvested autologously from a patient in box 3302. If required by a particular implant application, soft tissue may be ground or cut to a desired shape and configuration. If bone marrow is harvested, care may be taken to retain as much bone marrow and blood within the bone marrow during harvest and cutting operations. The harvested soft tissue is exposed to water to selectively lyse undesired cells types such as red blood cells, white blood cells, etc in box 3304. In some embodiments, ratios of tissue to water from 1 part bone to 1 part water and ranging to 1 part tissue to 200 parts water can be employed. Any remaining viable cells that are not attached to the tissue may be rinsed away in this fashion. Additionally, using a weak lysing agent (such as less then 1M acetic acid) may result in binding solubilized growth factors to the bone but still retaining viable progenitor cells attached to the bone.

The desired cells, such as adipose stem cells, apidocytes, mesenchymal stem cells, bone marrow stromal cells, progenitor cells, etc., remain viable in tissue. Other mechanical lysing techniques previously described, such as sonication, stirring induced shear, thermoslysis, etc., may be used in conjunction with the water bath to facilitate lysing of cellular material. After a lysing time (e.g., 1 minute-50 hours) has elapsed, saline is added to return osmolarity of the solution to physiological levels (e.g., approximately 0.9% salt) in box 3306. After the solution is returned to isotonic conditions, the fluid is decanted leaving the bone in box 3308. The effective rinse also facilitates removal of undesired cells unattached to the cancellous bone and discards them in the decanting step.

Antibiotics may be applied to the bone in box 3310 to help with decreasing bioburden levels. Alternatively, in some embodiments antibiotics can be administered to the harvested cancellous bone prior to the lysing step. Some antibiotics that may be used include gentamicin, vancomycin, amphotericin, other antibiotics previously mentioned or as can be appreciated, or various antibiotics that can be used to reduce bioburden in allograft tissues. After the reduction of bioburden, the bone may be exposed to storage or preservation fluids such as DMEM, DMSO, sucrose, mannitol, glucose, etc., in box 3312. The bone is then frozen until thawed for use in a surgical procedure to repair a skeletal defect. In some embodiments, the bone can be frozen at temperatures at or below −40 C.

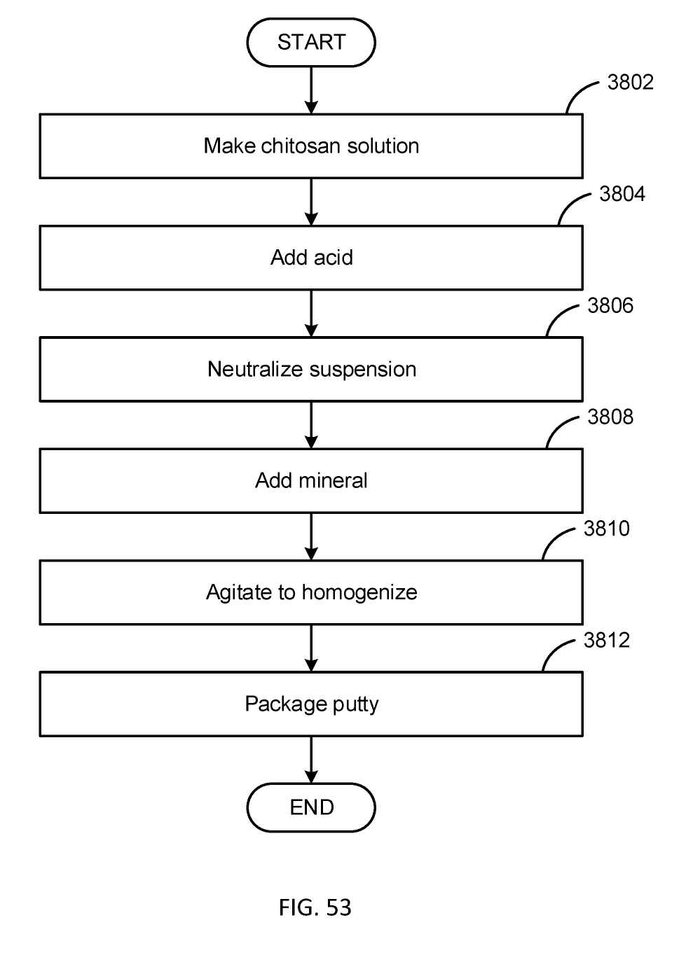

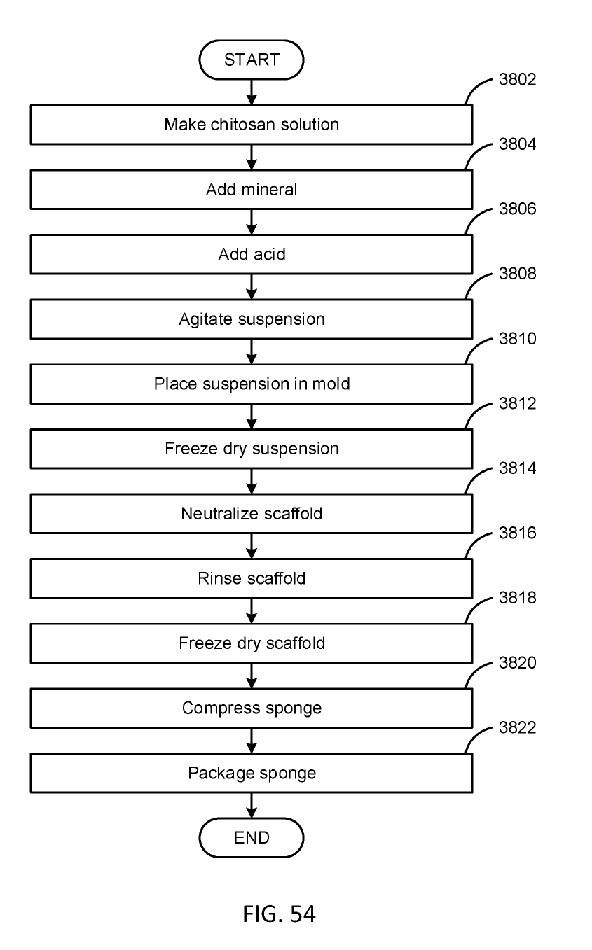

Reference is now made to FIG. 49, which depicts an alternative embodiment of the disclosure. In the depicted embodiment, the growth factors and bioactives obtained in the embodiments described above with reference to FIGS. 50 and/or 51 (as a non-limiting example) may be added to a biodegradable or resorbable polymer prior to dehydration. Accordingly, bone marrow harvested in box 3402 can be subjected to at least one filtration process in box 3404 as described above with reference to FIG. 46. The harvested bone marrow can be subjected to a lysing agent in box 906 as also described above.

In this embodiment, the growth factors and bioactives are harvested as previously described and added to a polymer with a common solvent, such as an acid. The biodegradable polymer may be a protein or polysaccharide, such as collagen, hyaluronan, chitosan, gelatin, etc., and combinations of two or more polymers. After the growth factors and bioactives are added to the polymer, it is mixed to obtain a substantially homogenous solution in box 3410. Any bubbles or impurities may then be removed from the substantially homogenous solution. If other materials (such as, but not limited to, calcium phosphate, hydroxyapatite, heparin, chondroitin sulfate, etc.) are desired to be embedded into the implant for growth factor attachment, degradation by products, and/or mechanical reinforcement, they can also be added to the mixture.

The mixture is frozen in box 3412 at a temperature that can range, in some embodiments, from −200 C to 0 C, to nucleate the water contained in the mixture into ice as well as condense the polymer/bioactive mixture into a porous structure. The mixture can be frozen in any geometry including, spherical, cylindrical, rectangular, in sheet form, tube form, etc. The implant will tend to retain this shape with its shape memory properties of the polymer is given space to expand in vivo. Temperatures can be increased to create larger pores or decreased to create small pores. Pores can be made directional by locating the cold temperature source substantially perpendicularly to the desired direction of the pores. Once the mixture is frozen at the desired temperature and pore direction, the implant is lyophilized and/or dehydrated in box 3414 to substantially eliminate the water contained within it. If acetic acid or another volatile substance was used as the solvent, that solvent will also be substantiailly eliminated by lyophilization.

After the lyophilization cycle is complete, the scaffold may be substantially neutralized in ethanol, saline, base, or buffer depending on the solvent used as a lysing agent in box 3415. In the case of an acetic acid solvent, the lyophilized implant may be rinsed in ethanol followed by saline or other rinsing agent in box 3416. After the saline rinse, the implant may be rinsed free of salts with water and vacuum dried or lyophilized to extend shelf-life. The dehydrated implants may be packaged under vacuum or sealed in vacuum sealed vials in box 3418. The implant can also be compressed prior to freezing and lyophilization or after neutralization and lyophilization to create a compacted scaffold that expands when exposed to fluid. Upon exposure to fluid, such an implant expands to substantially to approximately the original scaffold size. Delayed expansion may be achieved by compressing the neutralized scaffold and drying without freezing.

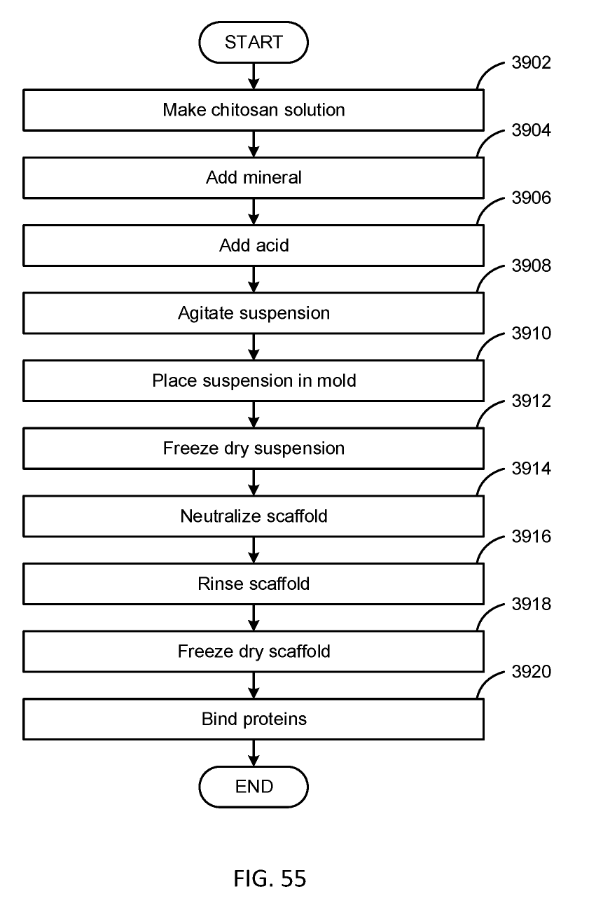

Reference is now made to FIG. 50, which depicts an alternative embodiment of the disclosure. In the depicted embodiment, the growth factors and/or bioactives obtained in the embodiments discussed with reference FIGS. 50 and 51 (as a non-limiting example) may be added to a biodegradable or resorbable polymer to create a flowable fluid and/or gel. In this embodiment, the growth factors and bioactives are harvested as previously described and added to a polymer with a common solvent, such as an acid. Accordingly, bone marrow harvested in box 3502 can be subjected to at least one filtration process in box 3504 as described above with reference to FIG. 46. The harvested bone marrow can be subjected to a lysing agent in box 3506 as also described above.

The biodegradable polymer may be a protein or polysaccharide, such as collagen, hyaluronan, chitosan, gelatin, etc., and combinations of two or more polymers. After the growth factors and bioactives are added to the polymer, it is mixed to obtain a substantially homogenous solution in box 3510. Any bubbles or impurities may be removed. If other materials (including, but not limited to, calcium phosphate, hydroxyapatite, heparin, chondroitin sulfate, etc.) are desired to be embedded into the implant for growth factor attachment, degradation by products, and/or mechanical reinforcement, they can also be added to the mixture.

A lysing agent can be chosen that is well tolerated by the body. For example, the growth factors and bioactives can be added to chitosan and in an acetic acid solution (0.01M-17M). The solution is mixed, and bubbles can be removed by applying vacuum or centrifugation. The gel can be packaged in syringes and either frozen and/or kept at ambient temperature in box 3512. Once injected and/or implanted into the body, the gel binds to tissue. Physiological fluids may buffer the gel to neutralize the pH and cause the gel to solidify in situ. Once the gel solidifies, the desired therapeutic implant remains in the intended surgical site and minimizes migration.

Reference is now made to FIG. 51, which depicts an alternative embodiment of the disclosure. A gel obtained as described in the above embodiment discussed with reference to FIG. 50 may be dehydrated using techniques such as vacuum drying, solvent evaporation, etc., to reduce the gel into a semi-rigid film and/or pellet. Accordingly, bone marrow harvested in box 3602 can be subjected to at least one filtration process in box 3604 as described above with reference to FIG. 46. The harvested bone marrow can be subjected to a lysing agent in box 3606 as also described above.

The gel is dehydrated as described above in box 3612. The pellets may be ground further or cut into the desired particle size depending on a desired implant application in box 3614. Once exposed to fluid and implanted into the surgical site, the pellets and/or powder resulting from ground pellets form a cohesive putty that can also bind to tissue. This binding property keeps the putty substantially in place at the surgical site when implanted. This putty can be used as a bioactive surgical adhesive. The application of such a putty may also be advantageous when used with autologous materials used in surgical procedures, such as autograft bone used in spinal fusion procedures, because it may be beneficial to help keep the autograft in a cohesive mass and minimize migration.

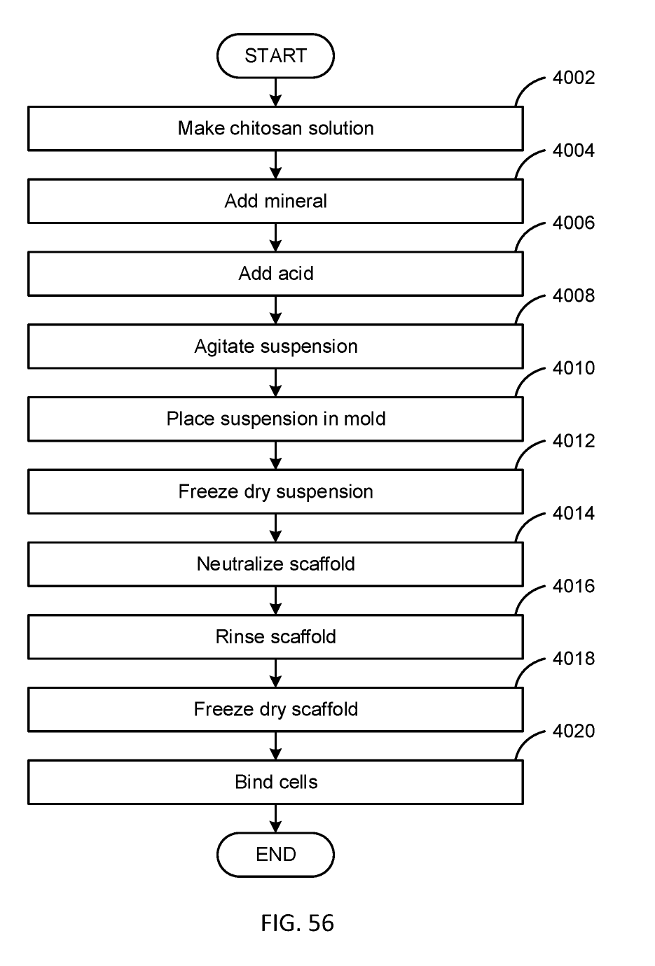

Referring now to FIG. 52, shown is a flow diagram illustrating a method to produce an embodiment of a low pH chitosan/mineral putty. In box 3702, a chitosan solution is made. The chitosan solution may be in the range of about 1% to about 25%. An acid (e.g., acetic acid) is then added in box 3704 to put the solution into a suspension. The acid may be in the range of about 0.1% to about 25%. A mineral in powder or granular form is then added in box 3706 and agitated to a homogenous mixture in box 3708. The putty is then packaged either wet or frozen in box 3710.

Referring next to FIG. 53, shown is a flow diagram illustrating a method to produce an embodiment of a neutral to partially neutral chitosan/mineral putty. In box 3802, a chitosan solution is made. The chitosan solution may be in the range of about 1% to about 25%. An acid (e.g., acetic acid) is then added in box 3804 to put the solution into a suspension. The acid may be in the range of about 0.1% to about 25%. The suspension is then neutralized or partially neutralized in box 3806 by adding base solution (e.g., sodium hydroxide or ammonium hydroxide) and agitating to homogenize the base solution. A mineral in powder or granular form is then added in box 3808 and agitated to a homogenous mixture in box 3810. The putty is then packaged either wet or frozen in box 3812.

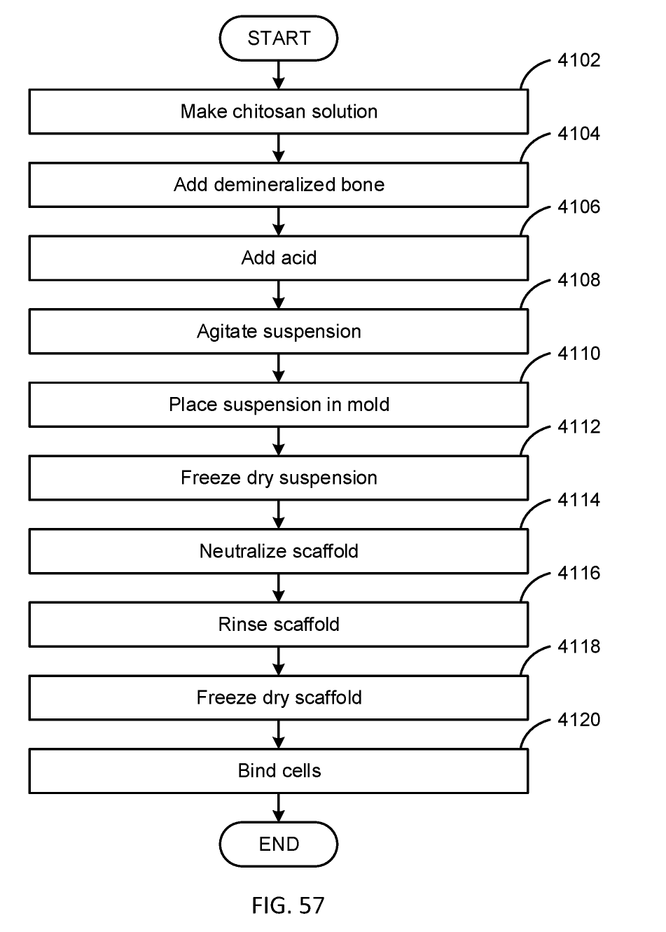

Referring now to FIG. 54, shown is a flow diagram illustrating a method to produce an embodiment of a neutral or partially neutral chitosan/mineral scaffold sponge. In box 3902, a chitosan solution is made. The chitosan solution may be in the range of about 1% to about 25%. A mineral in powder or granular form is then added in box 3904 and agitated to a homogenous mixture. An acid (e.g., acetic acid) is then added in box 3906 to put the solution into a suspension and agitated in box 3908. The acid may be in the range of about 0.1% to about 25%. The suspension is then placed into molds in box 3910 to conform to one or more desired shapes. The suspension is then freeze dried in box 3912. The molds are placed into a freezer and the suspensions are frozen to allow crystal formation. The frozen suspensions are lyophilized and the formed scaffolds are pulled out of molds. The scaffolds are then neutralized or partially neutralized in box 3914 by soaking in a base solution (e.g., sodium hydroxide or ammonium hydroxide). The scaffolds are then rinsed of any remaining base solution in sterile water or PBS in box 3916 and freeze dried in box 3918 where the scaffolds are frozen and lyophilized. The scaffolds are compressed into the desired shape in box 3920 and packaged and sterilized in box 3922.

Referring next to FIG. 55, shown is a flow diagram illustrating a method to produce another embodiment of a neutral or partially neutral chitosan/mineral scaffold sponge. In box 4002, a chitosan solution is made. The chitosan solution may be in the range of about 1% to about 25%. A mineral in powder or granular form is then added in box 4004 and agitated to a homogenous mixture. An acid (e.g., acetic acid) is then added in box 4006 to put the solution into a suspension and agitated in box 4008. The acid may be in the range of about 0.1% to about 25%. The suspension is then placed into molds in box 4010 to conform to one or more desired shapes. The suspension is then freeze dried in box 4012. The molds are placed into a freezer and the suspensions are frozen to allow crystal formation. The frozen suspensions are lyophilized and the formed scaffolds are pulled out of molds. The scaffolds are then neutralized or partially neutralized in box 4014 by soaking in a base solution (e.g., sodium hydroxide or ammonium hydroxide). The scaffolds are then rinsed of any remaining base solution in sterile water or PBS in box 4016 and freeze dried in box 4018 where the scaffolds are frozen and lyophilized. Proteins are then bound onto the scaffold by way of soaking or vacuum perfusion in box 4020.

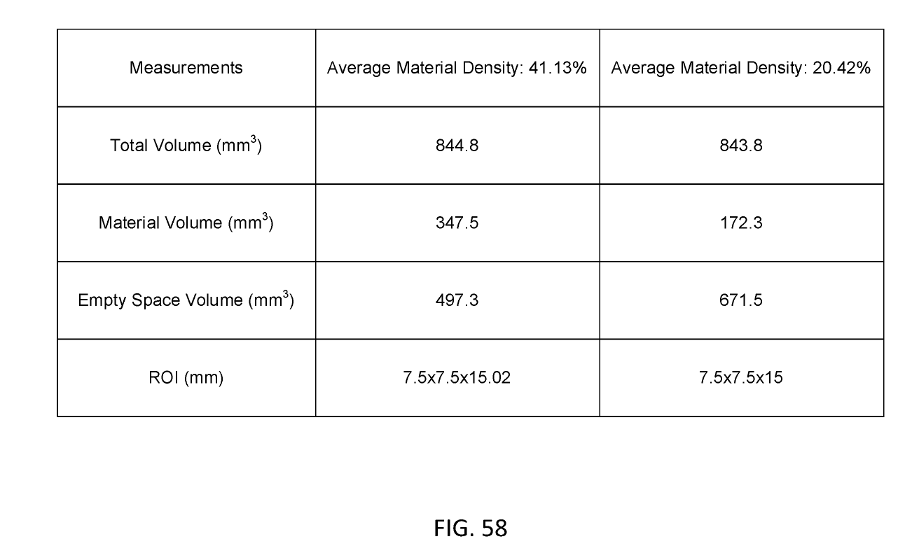

Reference is now made to FIG. 57, which depicts a flow diagram illustrating a method to produce an embodiment of a neutral or partially neutral chitosan/mineral scaffold sponge including seed cells. In box 4102, a chitosan solution is made. The chitosan solution may be in the range of about 1% to about 25%. A mineral in powder or granular form is then added in box 4104 and agitated to a homogenous mixture. An acid (e.g., acetic acid) is then added in box 4106 to put the solution into a suspension and agitated in box 4108. The acid may be in the range of about 0.1% to about 25%. The suspension is then placed into molds in box 4110 to conform to one or more desired shapes. The suspension is then freeze dried in box 4112. The molds are placed into a freezer and the suspensions are frozen to allow crystal formation. The frozen suspensions are lyophilized and the formed scaffolds are pulled out of molds. The scaffolds are then neutralized or partially neutralized in box 4114 by soaking in a base solution (e.g., sodium hydroxide or ammonium hydroxide). The scaffolds are then rinsed of any remaining base solution in sterile water or PBS in box 4116 and freeze dried in box 4118 where the scaffolds are frozen and lyophilized. Seed cells are then bound onto the scaffold by way of hydration, soaking or vacuum perfusion in box 4120.

Reference is now made to FIG. 57, which depicts a flow diagram illustrating a method to produce an embodiment of a neutral or partially neutral chitosan/demineralized bone scaffold sponge including seed cells. In box 4202, a chitosan solution is made. The chitosan solution may be in the range of about 1% to about 25%. Demineralized or partially demineralized bone in powder or granular form is then added in box 4204 and agitated to a homogenous mixture. An acid (e.g., acetic acid) is then added in box 4206 to put the solution into a suspension and agitated in box 4208. The acid may be in the range of about 0.1% to about 25%. The suspension is then placed into molds in box 4210 to conform to one or more desired shapes. The suspension is then freeze dried in box 4212. The molds are placed into a freezer and the suspensions are frozen to allow crystal formation. The frozen suspensions are lyophilized and the formed scaffolds are pulled out of molds. The scaffolds are then neutralized or partially neutralized in box 4214 by soaking in a base solution (e.g., sodium hydroxide or ammonium hydroxide). The scaffolds are then rinsed of any remaining base solution in sterile water or PBS in box 4216 and freeze dried in box 4218 where the scaffolds are frozen and lyophilized. Seed cells are then bound onto the scaffold by way of hydration, soaking or vacuum perfusion in box 4220. Once the cells are bound, the scaffolds may be packaged with a cryopreservative and frozen.

The following non-limiting embodiments are provided for further illustration.

### EXAMPLES

Now having described the embodiments of the present disclosure, in general, the following Examples describe some additional embodiments of the present disclosure. While embodiments of the present disclosure are described in connection with the following examples and the corresponding text and figures, there is no intent to limit embodiments of the present disclosure to this description. On the contrary, the intent is to cover all alternatives, modifications, and equivalents included within the spirit and scope of embodiments of the present disclosure.

### Example 1: Increased Growth Factors in Soft Tissue Implants Containing Adipose-Derived Intracellular Compounds

Introduction

Soft tissue implants made according to the methods described herein contain intracellular components, including growth factors such as vascular endothelial growth factor (VEGF), basic fibroblast growth factor (bFGF), and transforming growth factor beta 1 (TGFb1). In order to assess the growth factor content of the soft tissue implants described herein, adipose derived intracellular content was harvested and processed according to methods described herein and applied to an extracellular matrix. This composition is referred to as LipoAmp in this Example. The growth factor content of LipoAmp was compared to a control soft tissue implant as described in Brown, et al. 2011. Tissue Eng. Part C, 17:411-423.

Materials and Methods Briefly, subcutaneous fat was separated from the dermal layer of a subject. The harvested subcutaneous fat was ground via a blender to mechanically disrupt the cellular structure to form a mixture of hydrophilic and hydrophobic components. The hydrophilic and hydrophobic components were separated from one another based on their buoyancy. The hydrophobic portion, which contains inter alia the lipids, was discarded. Acetic acid (up to 50% v/v, e.g. about 25% v/v) was added to the hydrophilic fraction. The optional step of adding up to 1M HCl, was performed. Here, 0.6N HCl was added to the hydrophilic fraction. The resulting solution was then neutralized in phosphate buffered saline or NaOH as necessary. Excess liquids were removed via centrifugations.

Results

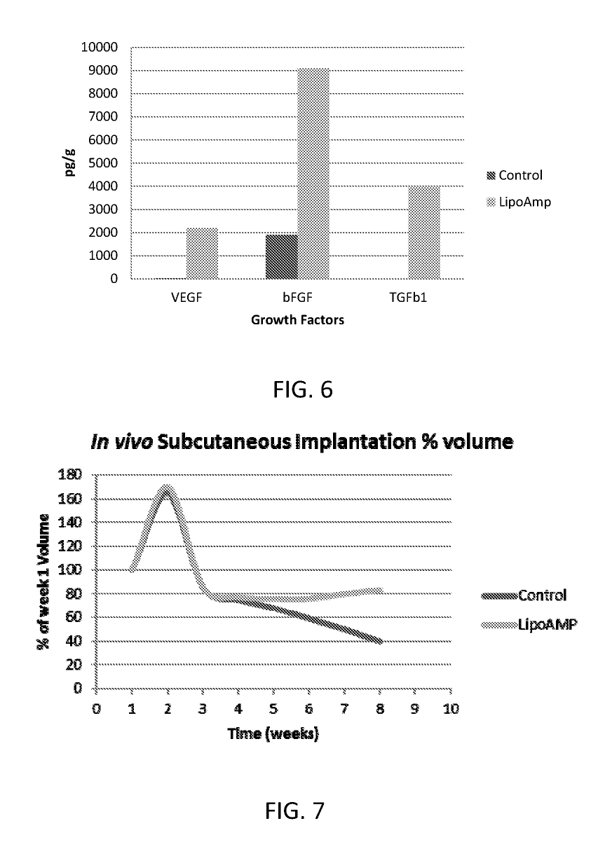

The results of this experiment are shown in FIG. 6, which demonstrates increased growth factor content in a carrier substrate combined with adipose-derived intracellular compounds (“LipoAmp”) as compared to control. Concentration (pg/g of implant) of the growth factors is shown on the y axis. The growth factors are shown on the x-axis. The soft tissue implant composition as described herein had a greater amount of VEGF, bFGF, and TGFb1.

### Example 2: Increased Adipose-Derived Soft Tissue Implantation Volume Compared to Native Tissue In Vivo

Introduction

The effect of a soft tissue implant made and administered according to the methods described herein (“LipoAmp”) on implant volume post implantation was examined in vivo.

Materials and Methods

LipoAmp was prepared as previously described in Example 1.

Results

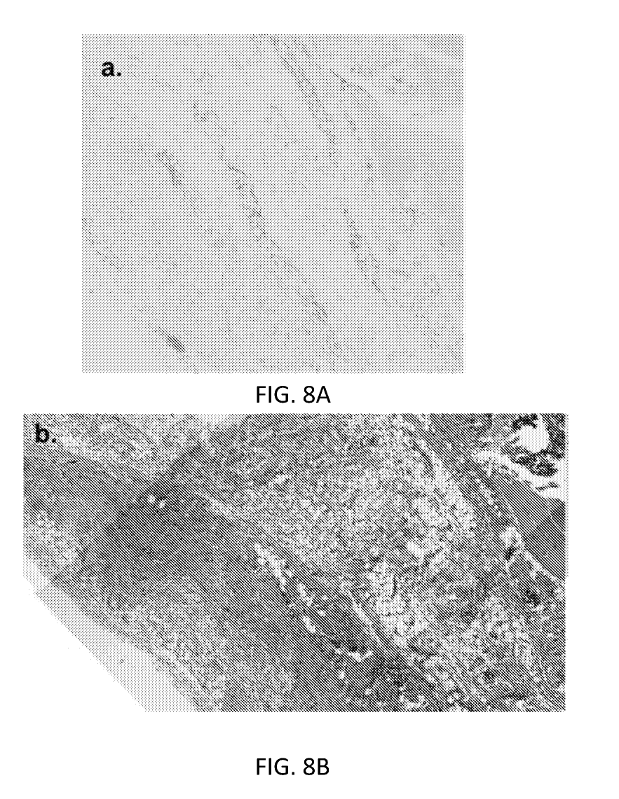

The results of this experiment are demonstrated in FIG. 7. As demonstrated by FIG. 7, while the Lipoamp implant and control maintained about the same volume, at about week 4, the performance of the two implants diverged. Over weeks 5 to 8, the Lipoamp implant maintained the volume at approximately 8 percent of the volume present at the start of the experiment. In contrast, the control implant decreased steadily in volume over weeks 5 to 8.

### Example 3: Soft Tissue Implant Containing Adipose-Derived Intracellular Compounds Induces Ectopic Adipogenesis In Vivo

Introduction

The effect of a soft tissue implant made and administered according to methods described herein (“LipoAmp”) on adipogenesis was examined in vivo.

Materials and Methods

To generate the LipoAmp, subcutaneous fat was separated from the dermal layer of a subject. The harvested subcutaneous fat was ground via a blender to mechanically disrupt the cellular structure to form a mixture of hydrophilic and hydrophobic components. The hydrophilic and hydrophobic components were separated from one another based on their buoyancy. The hydrophobic portion, which contains inter alia the lipids, was discarded. Acetic acid (up to 50% v/v, e.g. about 25% v/v) was added to the hydrophilic fraction. The optional step of adding up to 1M HCl, was performed. Here, 0.6N HCl was added to the hydrophilic fraction. The resulting solution was then neutralized in phosphate buffered saline or NaOH as necessary. Excess liquids were removed via centrifugations. The LipoAmp was then administered to a subject.

Results

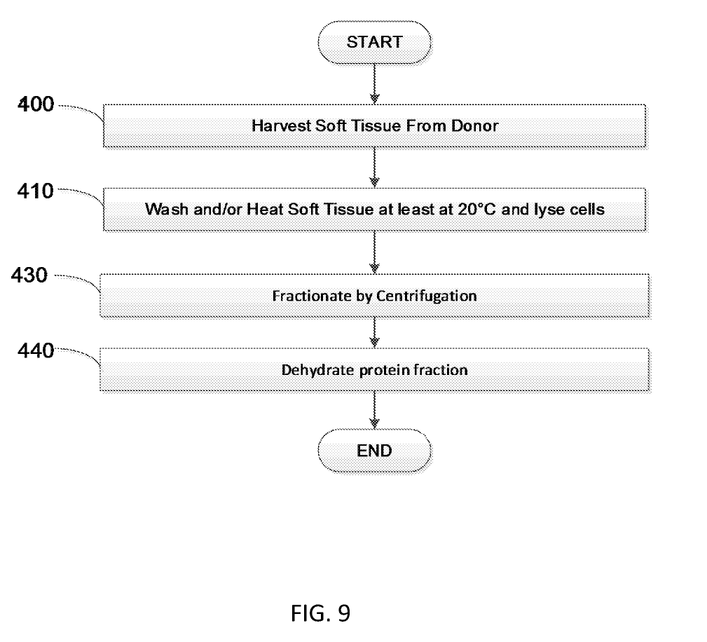

The results of this experiment are shown in FIGS. 8A and 8B. As demonstrated in FIG. 8B, adipogenesis is induced from the implant.

### Example

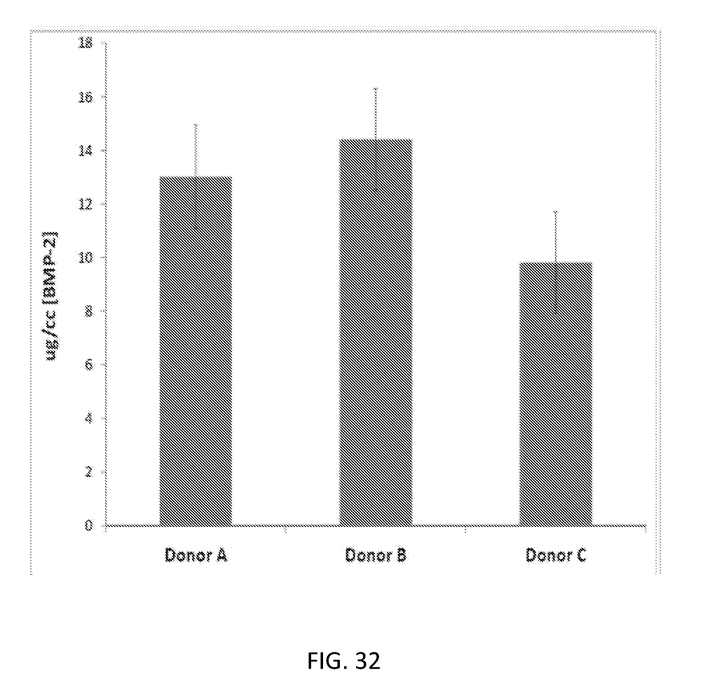

FIG. 31 demonstrates total protein concentration obtained by a method described herein. Total protein content was measured using bicinchoninic acid assay (BCA assay). The sample preparation involved reconstituting the dehydrated bone marrow protein composition with either water or saline. FIG. 31 therefore demonstrates the total protein in mg per cc of reconstituted sample soluble bone marrow protein compositions generated from 3 donors (A, B, and C). The testing was conducted according to the manufacturers' instructions (Pierce™ BCA Protein Assay Kit). The total protein concentration is exhibited by a color change of the sample solution from green to purple in proportion to protein concentration, which can then be measured using colorimetric techniques.

### Example 5

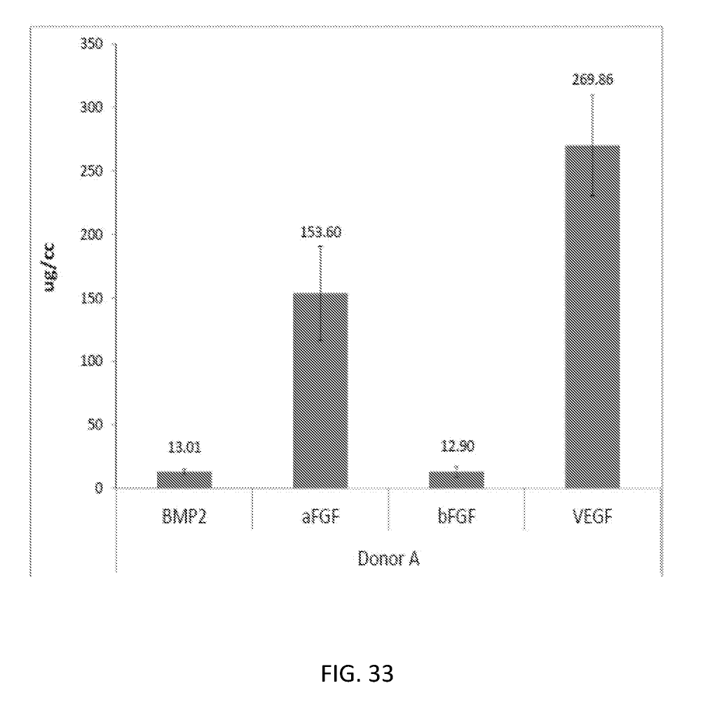

FIG. 32 demonstrates the concentration of BMP-2 protein as measured by an enzyme-linked immunosorbent assay (ELISA) in a soluble bone marrow compositions described herein derived from various bone marrow donors. Here BMP-2 protein was measured in reconstituted or extracted samples from 3 donors (A, B, C) Reconstitution is performed with either water or saline. Extractions are performed in different buffers (Guanidine-HCl or Urea-based buffers) in different concentrations for different incubation times. BMP-2 concentration is expressed as pg BMP-2 per cc of reconstituted or extracted samples.

### Example 6

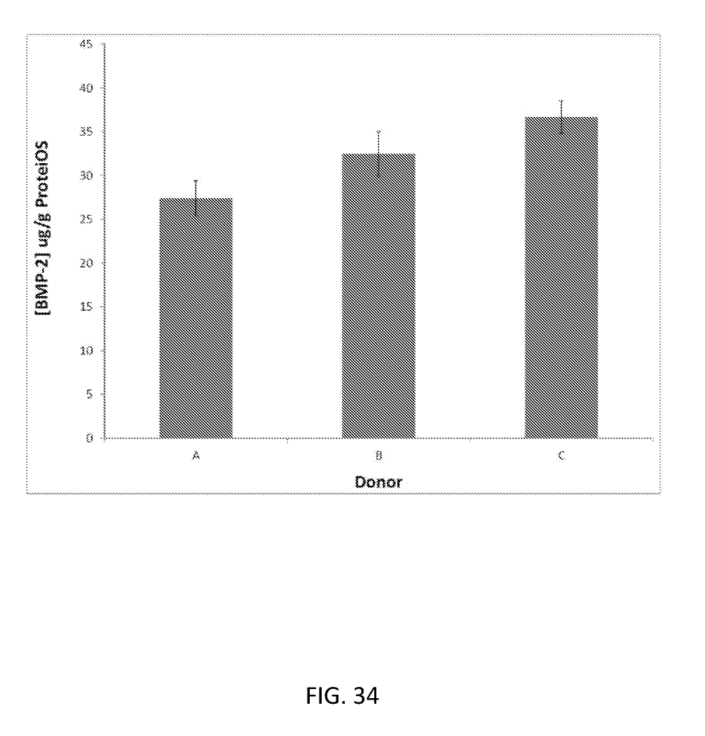

FIG. 33 demonstrates the concentration of various proteins present in a soluble bone marrow composition from various donors. The growth factors were quantified using ELISA. Test samples were either reconstituted or extracted from various donors (A, B, C). Reconstitution was performed with either water or saline. Extractions are performed in different buffers (Guanidine-HCl or Urea-based buffers) in different concentrations for different incubation times. Bioactive factor concentration is expressed as pg BMP-2 per cc of reconstituted or extracted samples.

### Example 7

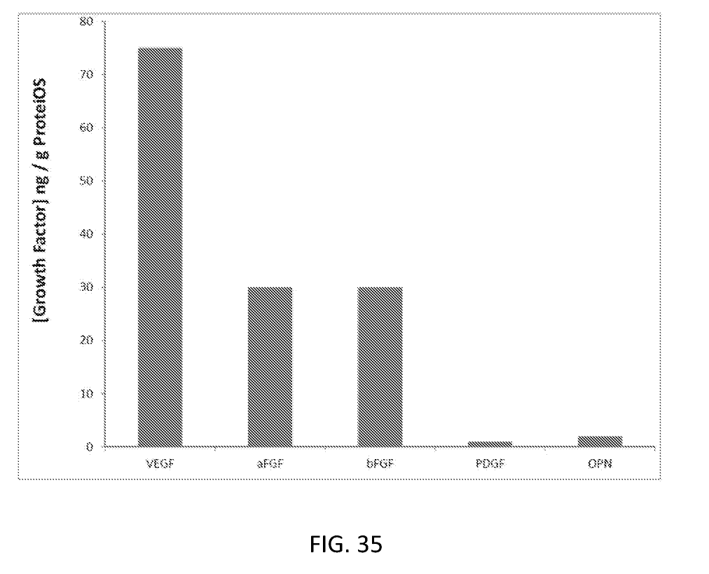

FIG. 34 demonstrates the concentration of BMP-2 ug/g of a soluble bone marrow protein composition (ProteiOS) from various donors.

### Example 8

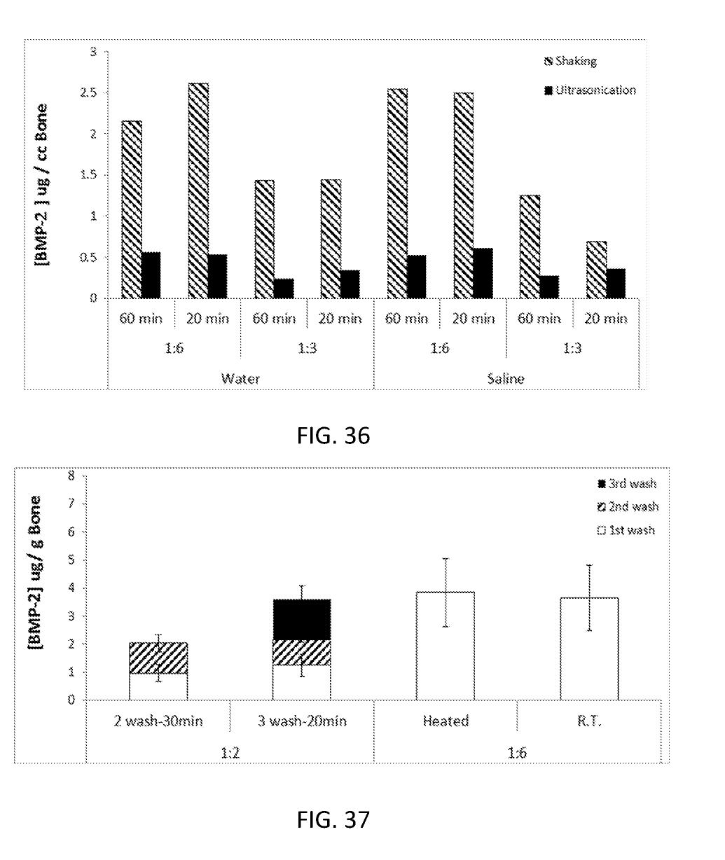

FIG. 35 demonstrates the concentrations of various bioactive factors (ng/g) of a soluble bone marrow protein composition (ProteiOS).

### Example 9

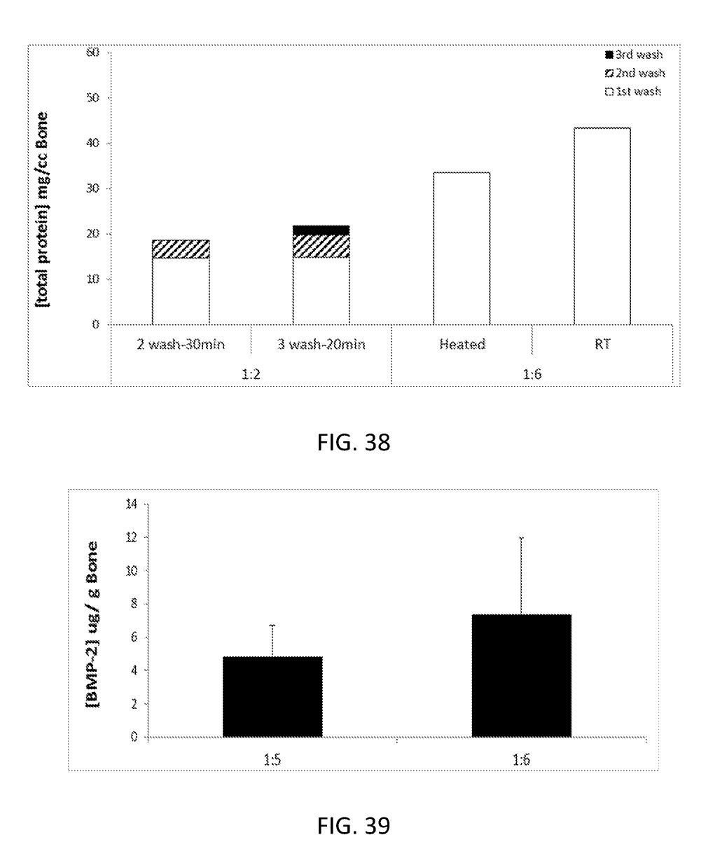

This Example examines the effect of processing time, bioactive factor processing methods (shaking or ultrasonication), processing time (about 20, 40, or 60 minutes) processing solution composition (water or a saline solution), processing temperature (37° C. or 25° C.), and ratio of starting bone material to processing solution (w/v) (1:3 or 1:6) on bioactive factor content in the final soluble bone marrow protein composition. About 3 grams of bone marrow containing material were processed according to the experimental design shown in Table 1. Briefly the starting material was washed in the processing solution at a particular ratio and incubated at a processing temperature and exposed to a processing method for an amount of time. BMP-2 content in the solution obtained was measured using an Enzyme-linked immunosorbent assays (ELISA). The results are demonstrated in FIG. 36.

TABLE 1

| Sample | Sample | Processing | | Processing | Processing | |
|---|---|---|---|---|---|---|
| number | ID | Solution | Ratio | Temp | Method | Time |
| 1 | 6W60S | Water | 1:6 | 37 | Shaking | 60 |
| 2 | 6W40S | | | | | 40 |
| 3 | 6W20S | | | | | 20 |
| 4 | 3W60S | | 1:3 | | | 60 |
| 5 | 3W40S | | | | | 40 |
| 6 | 3W20S | | | | | 20 |
| 7 | 6S60S | Saline | 1:6 | | | 60 |
| 8 | 6S40S | | | | | 40 |
| 9 | 6S20S | | | | | 20 |
| 10 | 3S60S | | 1:3 | | | 60 |
| 11 | 3S40S | | | | | 40 |
| 12 | 3S20S | | | | | 20 |
| 13 | 6W60S25 | Water | 1:6 | 25 | | 60 |
| 14 | 3W60S25 | | 1:3 | | | |
| 15 | 6S60S25 | Saline | 1:6 | | | |
| 16 | 3S60S25 | | 1:3 | | | |
| 17 | 6W60U | Water | 1:6 | 25 | Ultrasonicate | 60 |
| 18 | 6W40U | | | | | 40 |
| 19 | 6W20U | | | | | 20 |
| 20 | 3W60U | | 1:3 | | | 60 |
| 21 | 3W40U | | | | | 40 |
| 22 | 3W20U | | | | | 20 |
| 23 | 6S60U | Saline | 1:6 | | | 60 |
| 24 | 6S40U | | | | | 40 |
| 25 | 6S20U | | | | | 20 |
| 26 | 3S60U | | 1:3 | | | 60 |
| 27 | 3S40U | | | | | 40 |
| 28 | 3S20U | | | | | 20 |

### Example 10

In this Example, the effect of adding a rinsing step to the processing step was examined. The initial processing conditions were as follows: the ratio of the bone marrow containing starting material to processing solution was 1:2, the processing solution was water, and the processing conditions were a total of 60 minutes at 37° C. with shaking (See Example 9). Then one or two additional rinse steps were performed. The additional rinse steps can also be thought of as repeating the processing step. The experimental design is set forth in Table 2 and described below.

TABLE 2

| | Starting Material | Starting | |
|---|---|---|---|
| | (Marrow-rich | material:H₂O | |
| Sample | Bone) (g) | (preheated) | Rinse |
| A | 10 | 1:2 | Twice for 30 minutes |
| | | | each @ 37° C. |
| B | 10 | 1:2 | Thrice for 20 minutes |
| | | | each @ 37° C. |
| C | 10 | 1:6 | Once for 60 minutes |
| | | | @ 37° C. |

For the processing where one additional rinse (or processing) step was added (for a total of 2 washes or processing steps), the total incubation time was split into two 30 minute incubations, in which one incubation time corresponds to the initial processing step and the second incubation corresponds to the one additional rinse/processing step. For the processing where two additional rinses (or processing) steps were added (for a total of 3 washes or processing steps), the total incubation time was split into three 20 minute incubations, in which one incubation time corresponds to the initial processing step, one incubation time corresponds to the first additional rinse/processing step, and the third incubation time corresponds to the second additional rinse/processing step.

For each additional rinse, the resulting solution was collected from the processing or rinse step that preceded it. Then the same volume of fresh processing solution as the amount of resulting solution collected from the step that preceded it was added to the remaining material. The remaining material was incubated in the fresh processing solution for an additional 30 or 20 minutes (for the one additional or two additional rinses, respectively) at 37° C. with shaking, such that the total incubation time was about 60 minutes. The resulting solution after the final rinse/processing step was collected and maintained in a separate container.

For additional comparison, starting material containing bone marrow was processed using a single processing step using water as the processing solution at a ratio of 1:6. The processing method used was either shaking for 60 minutes at 37° C. or shaking at room temperature (about 25° C.) in deionized water that had been pre-warmed to 37° C.

The total protein, as measured using a BCA assay, and BMP-2 amount, as measured by ELISA, was measured in each of the collected solutions. The results are demonstrated in FIGS. 18-19.

### Example 11

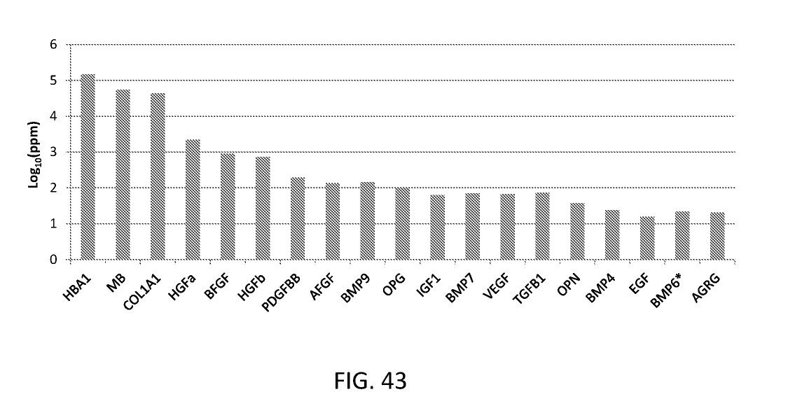

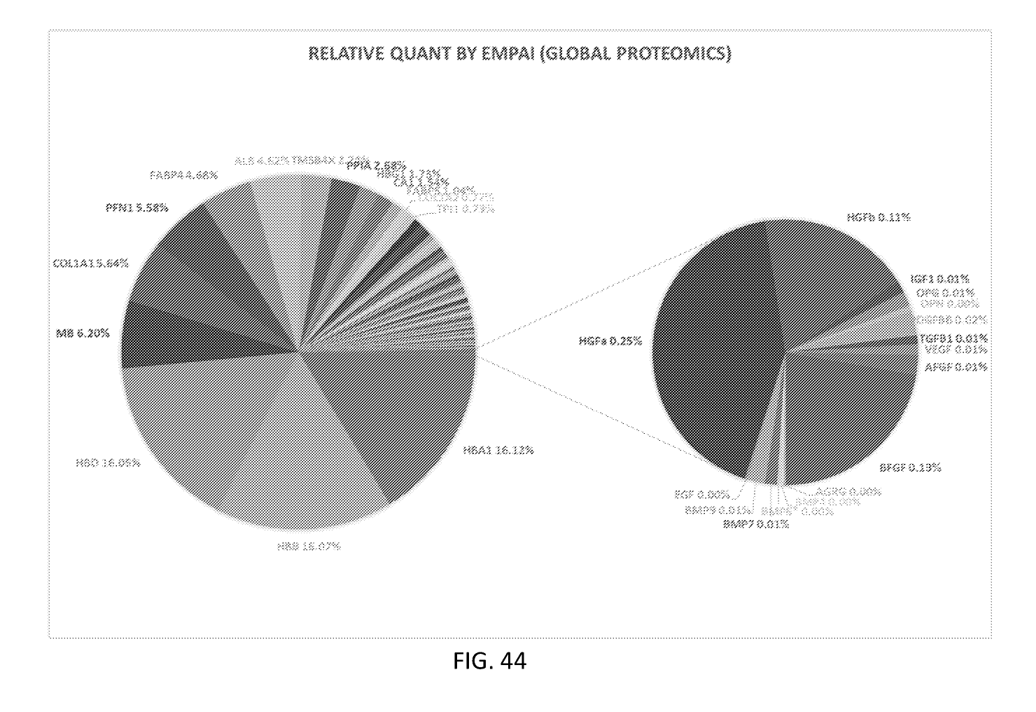

This Example evaluates the effect the ratio of starting material to processing solution on bioactive factor content in the soluble bone marrow protein composition. The processing of the bone marrow containing starting material was generally as described in Example 10 for the processing method that included one additional rinse/processing step except that the ratio of bone marrow containing starting material to water (w/v) was varied from 1:5 and 1:6, the starting material amount was about 12 g, the processing was conducted at 25° C. using pre-warmed (37° C.) water, and the solutions collected at each step were combined. The study design is presented in Table 3. The total protein, as measured using a BCA assay, and BMP-2 amount, as measured by ELISA, was measured in the final combined collected solution. The results are demonstrated in FIGS. 39-40.

TABLE 3

| | Starting | Starting Material | Rinse/Processing | Total |
|---|---|---|---|---|
| | Material | (Marrow-rich Bone)/ | step number and | Volume |
| Sample | (g) | Water (pre-warmed) | incubation time | (cc) |
| A | 12 | 1:5 incubated at 25° C. | 2x, 30 minutes | 60 |
| B | | 1:6 incubated at 25° C. | each | 72 |

### Example 12

This Example evaluates an optional filtering step using different combinations of filters. Several combinations were attempted including stacking different sized filters, using wet or dry filters. Observations and time for filtering (or clogging) were obtained. Briefly, the soluble mem Tables 4-5 show the study design and observational results. The filtration solution starting volume ranged from about 96 to 176 cc. All solutions were prepared from one lot of marrow-rich bone. When BMP-2 was evaluated by ELISA in the resulting solutions, it was observed that BMP-2 was present at a higher concentration. The BMP-2 concentration was measured to be about 33.68 pg/cc starting marrow-rich bone.

TABLE 4

| At- | | Filter | Dry | |
|---|---|---|---|---|
| tempt | Filter | Size | or | |
| # | Type | (μm) | Wet | Observations |
| 1 | Cellulose | 8 + 5 | Wet | fast easy, 5-10 seconds |
| | Acetate | stacked | | |
| | | 3 + 1.2 | | filtered in 1 min. 45 seconds, |
| | | stacked | | slowly |
| | | 0.8 | | fast, 20 seconds |
| | | 0.02 | Dry | fast, 20 seconds |
| 2 | Cellulose | 8, 5, 3 | Dry | total volume in 2.5 min. |
| | Acetate | stacked | | |
| | | 0.8 + 1.2 | | filtered in 1 min. 45 seconds, |
| | | stacked | | slowly |
| | | 1.2 | | filtered 110 mLs well in 30 |
| | | | | seconds |
| | | 0.8 | | slower than day before, 6 min. |
| | | 0.2 (twice) | | Tried 2 of them, both clogged at |
| | | | | 10 mLs |
| | PES | 0.45 | | meant to use 0.2, clogged at 20 |
| | | | | mLs |
| | Cellulose | 0.8 | Wet | filtered in 20 seconds |
| | Acetate | 0.2 | Dry | clogged at 10 mLs |

TABLE 5

| At- | | Filter | Dry | |
|---|---|---|---|---|
| tempt | Filter | Size | or | |
| # | Type | (μm) | Wet | Observations |
| 3 | Cellulose | 8 + 5 | Wet | total volume in 3 min. 25 seconds |
| | Acetate | stacked | | *heard air leak in unit |
| | | 3 | | 20 seconds |
| | | 1.2 | | 55 seconds |
| | | 0.8 (twice) | | 120 mL in 7 min. and clogged, rest |
| | | | | immediately (10-15 mL) |
| | | 0.2 | Dry | did not filter |
| | | 0.8 | Wet | 2 minutes |
| | | 0.8 | Wet | 35 seconds |
| | | 0.2 | Dry | ⅓ volume in 2 min, clogged |
| | | 0.2 | | little more than ⅓ in 3 minutes, |
| | | | | clogged |
| | | 0.2 | | 45 sec to filter remaining |
| 4 | Cellulose | 8 + 5 + 3 | Wet | didn't filter |
| | Acetate | stacked | | |
| | | 8 + 5 + 3 | Dry | slow, 75-100 mLs in 2-3 min. |
| | | stacked | | |
| | | 8 + 5 | | filtered remainig volume easily |
| | | 3 | | 55 seconds |
| | | 1.2 | Wet | 85 seconds |
| | | 0.8 | | 30 seconds |
| | | 0.2 | Dry | well for 45 seconds, then last 5-10 |
| | | | | mLs in 30 seconds |

### Example 13

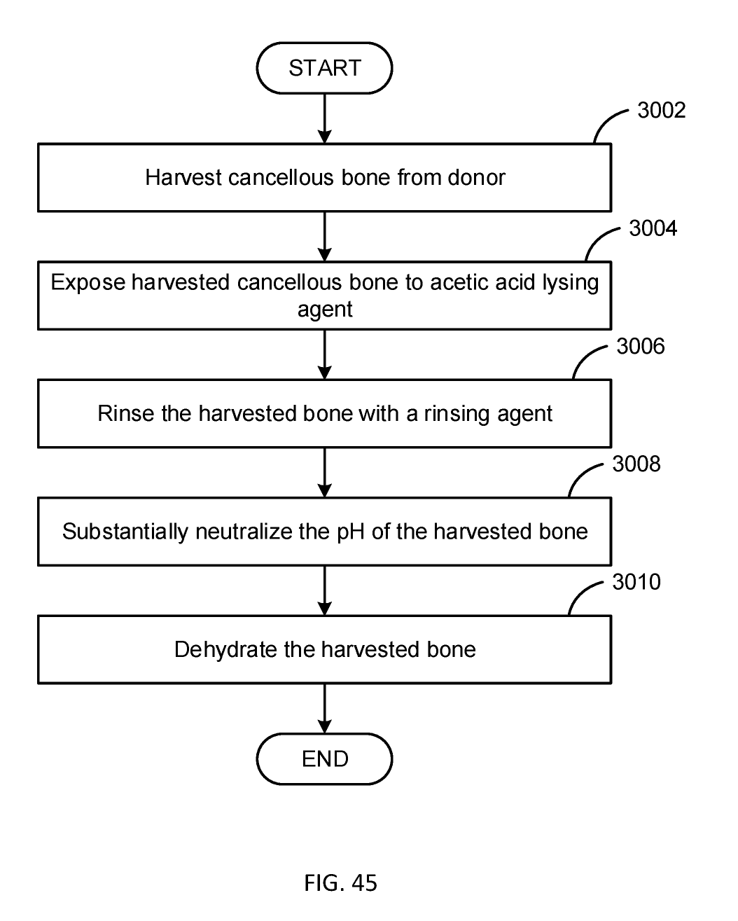

This Example evaluated the effect of including an optional filtering step performed on a large volume (about 1250 cc) of starting volume of processing solution. About 225 g of bone marrow containing granules were processed in 1250 mL of water. The processing observations are shown in Table 6. As shown in FIG. 41, a considerable about of BMP-2 was present in the final soluble bone marrow protein composition and averaged about 5 pg/cc of starting bone marrow containing material.

TABLE 6

| Filter Size (μm) | Observations |
|---|---|
| 8 + 5 (Stacked) | Clogged @ 45 sec |
| | Clogged @ 45 sec |
| 8 | Slowed at 1 min, 180 mL in 2 min |
| | Slowed at 1 min, 120 mL in 2 min |
| | Sowed at 1 min, 140 mL in 2 min |
| | Slowed @ 1 min, 160 mL in 2 min |
| | Slowed @ 1 min, 150 mL in 1 min |
| | Filtered remaining in 40 sec. |
| 5 | Fast, easy, total volume in 40 sec. |
| 3 | Slowed at 5.5 min, filtered total in 7.5 min |
| 1.2 | Immediately slow, 80 mL in 1 min. |
| | Slowly, about 700 mL in 14 min. |
| 0.8 | 8 filters, each clogged around 2 min, each |
| | filtered around 130 mL |
| 0.2 | 6 filters total |

### Example 14

This Example evaluates the effect of different stabilizer components and their effect on the binding of components of the soluble membrane protein composition to different graft scaffolds (e.g. VITOSS material, demineralized cortical bone, and mineralized cortical bone. The 1× stabilizer formulation contained (per 100 mL) 100 mg sucrose, 500 mg glycine, 370 mg glutamic acid, 2 mg NaCl, and 2 mg Polysorbate-80. Glutamic acid was varied in the stabilizer solution and was substituted in some instances with other mild acids such as acetic acid. The stabilizer component variations were as follows: (1) with glutamic acid 1×; (2) with glutamic acid 2.5×; (3) with glutamic acid 5×; (4) without glutamic acid but with 160 μL of 10% acetic acid; (5) without glutamic acid but with 320 μL of 10% acetic acid; (6) with glutamic acid 2.5×+160 μL of 10% acetic acid; (7) without glutamic acid but with 40 μL of 0.6N HCl; (8) without glutamic acid but with 80 μL of 0.6N HCl; and (9) with glutamic acid 2.5× and 40 μL of 0.6N HCl. Bound bioactive factors were indirectly determined by determining the amount of unbound bioactive factors remaining.

General processing parameters are shone in Table 7. Briefly, bone marrow containing starting material was weighed and processed in about 98 mLs of pre-warmed (37° C.) water for about 30 minutes. The solution was collected and fresh pre-warmed water was added to the bone marrow containing starting material and processed as before. The solution was collected and combined with the solution collected from the first step. The combined solution was stored at about 4° C. for about 2 hours. The chilled solution was cleaned by filtering and centrifugation as set forth in Table 7. 140 mLs was recovered after the cleaning filtration. The 140 mLs were divided into 9 aliquots and each aliquot was mixed with a different stabilizer from the stabilizer variations 1-9 previously described. Each of the 9 samples were then divided into 5 mL aliquots and frozen overnight at −80° C. Then, the samples were lyophilized.

Lyophilized samples from stabilizer variations 1, 3, 5, 6, 8, and 9 were reconstituted in 1 mL deionized water and duplicates were combined. 500 μL of the reconstituted sample was added to VITOSS material and incubated for about 15 minutes with no agitation at about 25° C. The liquid was collected and passed through a 100 μM nylon filter. This process was repeated using demineralized cortical bone or mineralized cortical bone instead of VITOSS material.

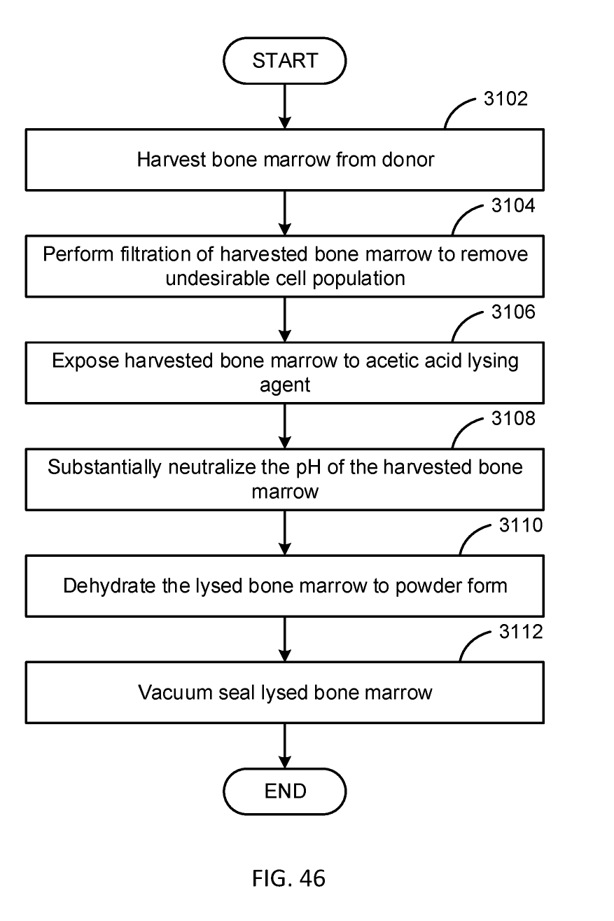

The reconstituted samples, the filtrate liquid, from all materials processed were lyophilized again and were incubated in 4M guanidine-HCl (Gu-HCl) pH 5.8 with shaking at 37° C. The amount of 4M Gu-HCl is based on the pre-lyophilized volume. The pH of the stabilizer solutions before and after reconstitution are shown in Table 8. For every 1 mL of sample volume, about 500 mL is used. Here the reconstituted samples prior to re-lyophilizing them ranged from about 140 μL to about 300 μL and the amount of 4M Gu-HCl was scaled to these amounts using the 1 mL:500 mL sample volume ratio. After incubating, samples were diluted 6× in 4M Gu-HCl pH 5.8 in duplicate and shaken at 25° C. for 1 hour. Samples were diluted 5×, 10×, and 25× in a calibrator diluent and tested for bioactive factors on Antigenix plates for evaluation of BMP-2 using ELISA. The % unbound BMP-2 is shown in FIG. 42.

Samples 1, 3, 5, 6, 8, and 9 were diluted in water at 1×, 10×, 25×, 50×, 100×, and 200× and total protein was evaluated using a BCA assay. The results of the total protein is not shown.

TABLE 7

| | | Granules | Ratio w/pre- | | Extraction | | Cleaning | | |
|---|---|---|---|---|---|---|---|---|---|
| Donor | Sample | (g) | warmed H2O | Rinse | Filtration | Ctfg. | Filtration | Stabilizer | Extraction |
| Lifelink - | A | 39.26 | 1:5 shaken at | 2x, 30 | 106, 75, | 1000 g, | 8, 5, 3, | Sample mixed | 500 mL 4M |
| TNS- | | | 25 C. (196.30 mLs | minutes | 53 uM | 2 min. | 1.2, 0.2 uM | with stabilizer | Gu-HCl pH |
| 0202110001- | | | total) | each | seives | | cellulose | variations as | 5.8, 37 C., |
| 15 | | | | | | | acetate | described then | shaking, 24 |
| | | | | | | | | lyophilized | hours |

TABLE 8

| | Stabilizer Formulation | | | | | | | | | |
|---|---|---|---|---|---|---|---|---|---|---|
| | 0 (stabilizer without glutamic acid) | 1 | 2 | 3 | 4 | 5 | 6 | 7 | 8 | 9 |
| pH after | 5.75 | 4.05 | 3.81 | 3.66 | 3.66 | 3.81 | 3.66 | 3.95 | 3.68 | 3.39 |
| preparing | | | | | | | | | | |
| solution | | | | | | | | | | |
| pH after | N/A | 4.5 | N/A | 4 | N/A | 4.5-5.0 | 4 | N/A | 7 | 4 |
| lyo'd and | | | | | | | | | | |
| reconstituted | | | | | | | | | | |
| In 1 mL | | | | | | | | | | |

### Example 15

Adipose tissue was exposed to lysing agent (saline or water), frozen, cut to shape/ground, and the water soluble fraction was isolated. Proteins were purified using centrifugation and filtration, and then a stabilizer/storage agent was added prior to lyophilization. The implant can be injected or otherwise administered to a subject in this form or combined with a delivery enhancer or carrier to ease delivery via implantation, injection, or transdermally.

TABLE 9
Select Growth Factor Concentrations Quantified
Using ELISA (per 500 g processing run):

| | aFGF | bFGF | VEGF |
|---|---|---|---|
| | (ug) | (ug) | (ug) |
| | w13-199 | | |
| Cellular Adipose | 3.38 | 5.53 | 1.89 |
| Lysate Solution | 1.01 | 0.25 | 0.06 |
| Accelular Adipose | 0.16 | 0.04 | 0.01 |
| Free Unbound | 1.96 | 0.07 | 1.19 |
| Protein Solution | | | |
| | w13-362 | | |
| Cellular Adipose | 11.39 | 1.41 | 0.48 |
| Lysate Solution | 1.02 | 0.28 | 0.03 |
| Accelular Adipose | 0.12 | 0.03 | 0 |
| Free Unbound | 2.16 | 0.08 | 1.5 |
| Protein Solution | | | |
| | w13-328 | | |
| Cellular Adipose | 4.17 | 3.36 | 1.27 |
| Lysate Solution | 0.94 | 0.25 | 0.04 |
| Accelular Adipose | 0.08 | 0.03 | 0 |
| Free Unbound | 2.88 | 0.05 | 1.35 |
| Protein Solution | | | |

Additional growth factors tested and present in the implant were Angiogenin, ANG-2, EGF, bFGF, HB-EGF, HGF, Leptin, PDGF-BB, PIGF, VEGF, IGF-I, IL-1b, IL-6, IL-8, Insulin, Leptin, MCP-1, PAI-1, Resistin, and TNFa.

### Example 16

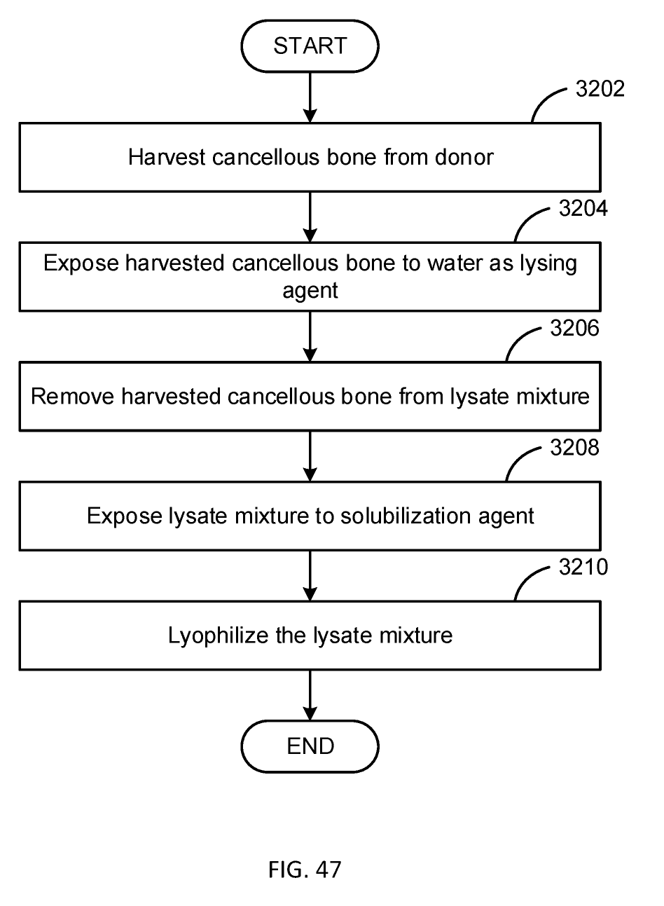

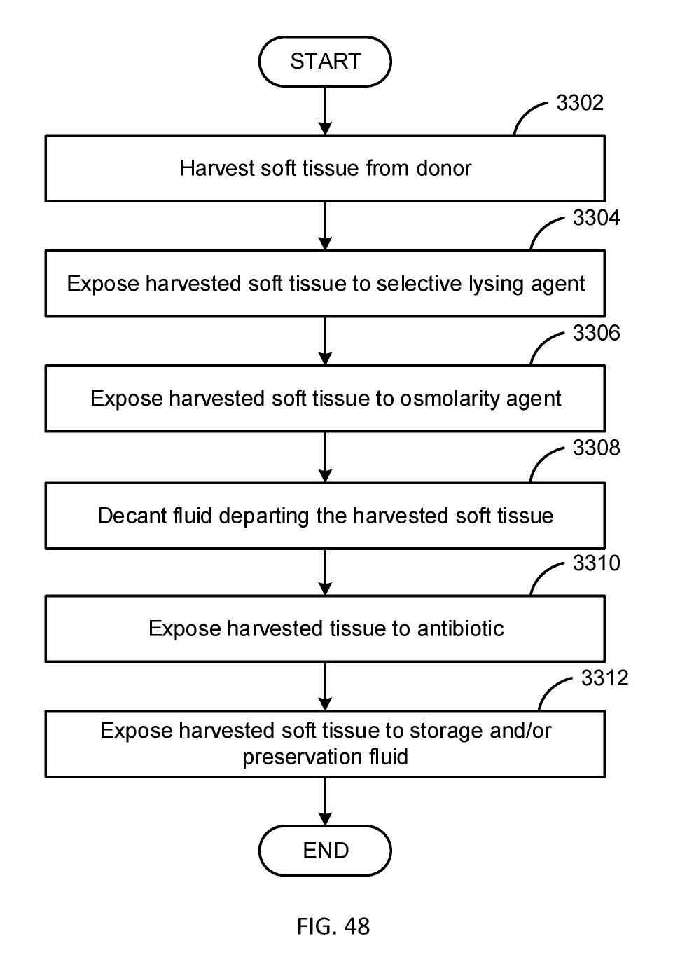

Bone marrow was obtained and cells were lysed and proteins solubilized in water. Protein solution was centrifuged, filtered, and a stabilizer was added prior to lyophilization. This soluble power may be reconstituted with water/saline and injected or added to a delivery enhancer such that proteins could be delivered transdermally. Microneedling, microrollering, or other perforation/abrasion techniques may also aid in delivery. FIG. 43 shows a sample of proteins identified with mass spectrometry. Other proteins are listed below (the relative quantification of some are shown in FIG. 44):

### Example 17

Patients can beassessed for hair loss or poor hair quality/health. Initial follicle density, shaft diameter, and overall hair quality will be measured. Patients can then receive implants that are injected/microneedled into their scalp. The physician may also include other treatments post injection (such as light or supplement therapies). After 3-6 months, the patients can be assessed again for follicle density, shaft diameter, and overall hair quality. Patients can also rate their own satisfaction with the results of the treatment.

### Example 18

Scaffold Sponge Formulation—Percent by Mass (Parts/100 Parts)

In a non-limiting example, a solution of 6% of greater than 300 kDa molecular weight chitosan solution (>75% deacetylation) mixed in with 6% of tri-calcium phosphate (TCP) in 83.6% water was initially created. The solution was then mixed in with 4.4% of acetic acid to put the solution into suspension. The suspension was then placed into molds and frozen at a controlled rate by a ramp of 5° C. every 15 minutes to a temperature of −80° C. Once the suspension turned to a solid, the molds were lyophilized until drying was completed. The scaffolds were then hydrated with a 2 molar NaOH solution. Scaffolds were then rinsed with sterile water until reaching a neutral pH. Scaffolds were then frozen at a controlled rate and freeze dried to until dry.

### Example 19

In a non-limiting example, a solution of 4% of greater than 300 kDa molecular weight chitosan solution (>75% deacetylation) mixed in with 6% of TCP in 85.6% water was initially created. The solution was then mixed in with 4.5% of acetic acid to put the solution into suspension. The suspension was then placed into molds and frozen at a controlled rate by a ramp of 5° C. every 15 minutes to a temperature of −80° C. Once the suspension turned to a solid, the molds were lyophilizeduntil drying was completed. The scaffolds were then hydrated with a 2 molar NaOH solution. Scaffolds were then rinsed with sterile water until reaching a neutral pH. Scaffolds were then frozen at a controlled rate and freeze dried to till dry.

### Example 20

In a non-limiting example, a solution of 3% of greater than 300 kDal molecular weight chitosan solution (>75% deacetylation) mixed in with 6% parts of TCP in 86.45% water was initially created. The solution was then mixed in with 4.55% of acetic acid to put the solution into suspension. The suspension was then placed into molds and frozen at a controlled rate by a ramp of 5° C. every 15 minutes to a temperature of −80° C. Once the suspension turned to a solid, the molds were lyophilized until drying was completed. The scaffolds were then hydrated with a 2 molar NaOH solution. Scaffolds were then rinsed with sterile water until reaching a neutral pH. Scaffolds were then frozen at a controlled rate and freeze dried to until dry.

### Example 21

In a non-limiting example, a solution of 2% of greater than 300 kDa molecular weight chitosan solution (>75% deacetylation) mixed in with 6% of TCP in 87.4% water was initially created. The solution was then mixed in with 4.6% of acetic acid to put the solution into suspension. The suspension was then placed into molds and frozen at a controlled rate by a ramp of 5° C. every 15 minutes to a temperature of −80° C. Once the suspension turned to a solid, the molds were lyophilized unit drying was completed. The scaffolds are then hydrated with a 2 molar NaOH solution. Scaffolds were then rinsed with sterile water until reaching a neutral pH. Scaffolds are then frozen at a controlled rate and freeze dried to until dry.

### Example 22

Sponge Formulation with Protein—Percent by Mass (Parts/100 Parts)

In a non-limiting example, a solution of 3% of greater than 300 kDa molecular weight chitosan solution (>75% deacetylation) mixed in with 6% parts of TCP in 86.45% water was initially created. The solution was then mixed in with 4.55% of acetic acid to put the solution into suspension. The suspension was then placed into molds and frozen at a controlled rate by a ramp of 5° C. every 15 minutes to a temperature of −80° C. Once the suspension turned to a solid, the molds were lyophilized until drying was completed. The scaffolds were then hydrated with a 2 molar NaOH solution. Scaffolds were then rinsed with sterile water until reaching a neutral pH. Scaffolds were then frozen at a controlled rate and freeze dried to until dry. The scaffolds were then fully saturated with protein solution.

### Example 23

Sponge Formulation with Cells—Percent by Mass (Parts/100 Parts)

In a non-limiting example, a solution of 3% of greater than 300 kDa molecular weight chitosan solution (>75% deacetylation) mixed in with 6% parts of TCP in 86.45% water was initially created. The solution was then mixed in with 4.55% of acetic acid to put the solution into suspension. The suspension was then placed into molds and frozen at a controlled rate by a ramp of 5° C. every 15 minutes to a temperature of −80° C. Once the suspension turned to a solid, the molds were lyophilized until drying was completed. The scaffolds were then hydrated with a 2 molar NaOH solution. Scaffolds were then rinsed with sterile water until reaching a neutral pH. Scaffolds were then frozen at a controlled rate and freeze dried to until dry. The scaffolds were then fully saturated with a physiological fluid containing viable cells.

### Example 24

Acidic Putty Formulation—Percent by Mass (Parts/100 Parts)

In a non-limiting example, a solution of 1% of greater than 300 kDa molecular weight chitosan solution (>75% deacetylation) mixed in 45% water was initially created. The solution was then mixed in with 1% of acetic acid to put the solution into suspension. 53% of TCP was then added into the suspension and agitated until a homogeneous mixture was reached.

In a non-limiting example, a solution of 1% of greater than 300 kDa molecular weight chitosan solution (>75% deacetylation) mixed in 44% water was initially created. The solution was then mixed in with 2% of acetic acid to put the solution into suspension. 53% of TCP was then added into the suspension and agitated until a homogeneous mixture was reached.

In a non-limiting example, a solution of 1% of greater than 300 kDa molecular weight chitosan solution (>75% deacetylation) mixed in 43% water was initially created. The solution was then mixed in with 3% of acetic acid to put the solution into suspension. 53% of TCP was then added into the suspension and agitated until a homogeneous mixture was reached.

### Example 25

Neutral Putty Formulation—Percent by Mass (Parts/100 Parts)

In a non-limiting example, a solution of 1% of greater than 300 kDa molecular weight chitosan solution (>75% deacetylation) mixed in 45% water was initially created. The solution was then mixed in with 1% of acetic acid to put the solution into suspension. The suspension was then neutralized with 3% 2 molar NaOH solution and agitated. 53% of TCP was then added into the suspension and agitated until a putty-like consistency was reached.

### Example 26

Putty Formulation with Protein—Percent by Mass (Parts/100 Parts)

In a non-limiting example, a solution of 1% of greater than 300 kDa molecular weight chitosan solution (>75% deacetylation) mixed in 45% water was initially created. The solution was then mixed in with 1% of acetic acid to put the solution into suspension. The suspension was then neutralized with 3% 2 molar NaOH solution and agitated. 53% of TCP was then added into the suspension and agitated until a putty-like consistency was reached. The putty was then fully saturated with a protein solution.

### Example 27

Putty Formulation with Cells—Percent by Mass (Parts/100 Parts)

In a non-limiting example, a solution of 1% of greater than 300 kDa molecular weight chitosan solution (>75% deacetylation) mixed in 45% water was initially created. The solution was then mixed in with 1% of acetic acid to put the solution into suspension. The suspension was then neutralized with 3% 2 molar NaOH solution and agitated. 53% of TCP was then added into the suspension and agitated until a putty-like consistency was reached. The putty was then fully saturated with a physiological fluid containing viable cells.

### Example 28

Granular Powder Formulation—Percent by Mass (Parts/100 Parts)

In a non-limiting example, a solution of 2% of greater than 300 kDa molecular weight chitosan solution (>75% deacetylation) mixed in 45% water was initially created. The solution was then mixed in with 2% of acetic acid to put the solution into suspension. 51% of TCP was then added into the suspension and agitated until a putty-like consistency was reached. The putty was lyophilized and ground into a powder. The powder was mixed with autograft bone or a physiological fluid intraoperatively to create a gel or putty. The granular powder may be maintained as a powder for later reconstitution.

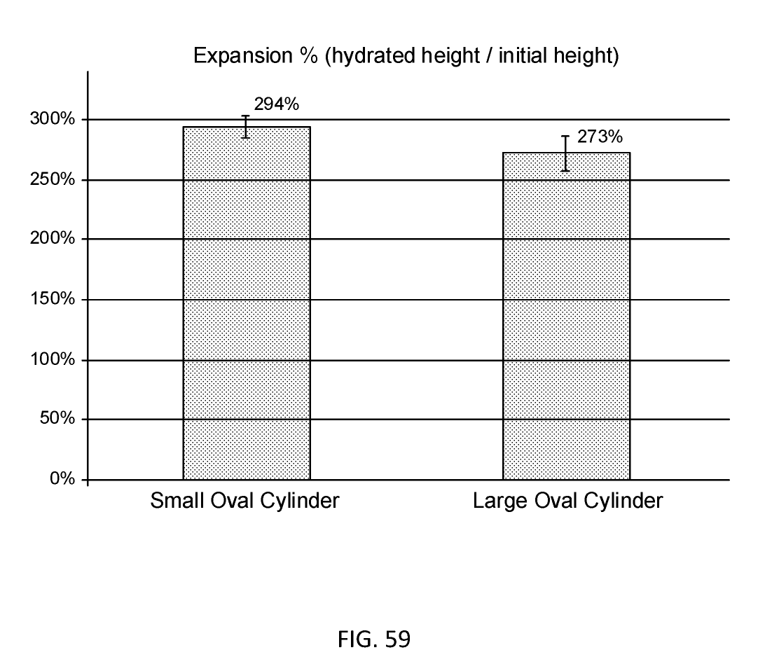

The chitosan/TCP scaffolds exhibited a porosity ranging from about 20 to about 80 μm. FIG. 58 provides examples of material properties of 41.13% and 20.42% material density scaffolds including volume of, material volume, empty space volume, and ROI.

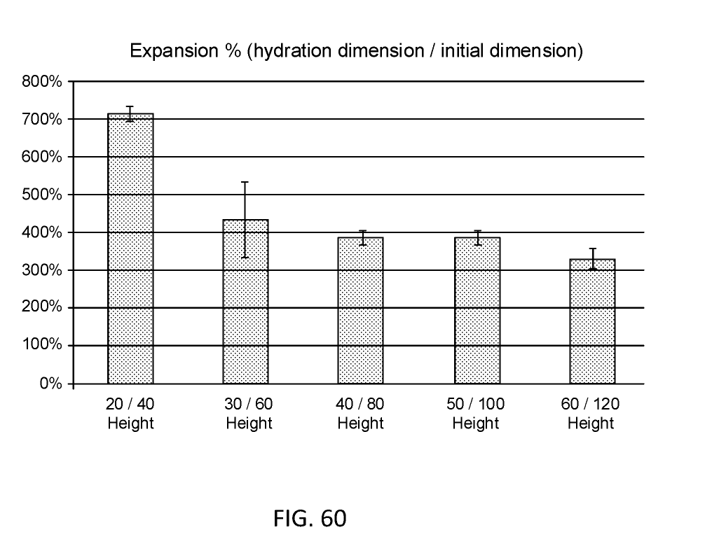

Referring next to FIG. 59, shown is a graph for circumferential expansion in accordance with an exemplary embodiment of a scaffold. In this embodiment, the hydrated dimension was compared to the compressed dimension of the scaffold and the total expansion percentage was calculated based on a 30 mg/mL chitosan with 60 mg/mL TCP formulation.

Referring next to FIG. 60, shown is a graph for uniaxial expansion in accordance with an exemplary embodiment of a scaffold. In this embodiment, the hydrated dimension was compared to the compressed dimension of the scaffold. Total expansion percentages were calculated for different formulations including chitosan concentrations of 20, 30, 40, 50, and 60 mg/mL corresponding to tri-calcium phosphate concentrations of 40, 60, 80, 100, and 120 mg/mL, respectively.

### Example 29

Table 10 below demonstrates concentrations of bioactive intracellular components of an embodiment of a tissue implant according to the present disclosure as described herein (AMP) prepared by methods as described herein (far right column) compared to traditional tissue implants prepared by traditional kits. The values listed for AMP demonstrate an embodiment of an effective amount to deliver to subjects in need thereof according to embodiments of the present disclosure.

| | | | | | | | | [symbol] | | | Discontinous | |
|---|---|---|---|---|---|---|---|---|---|---|---|---|
| | | | | | | | | [symbol] | [symbol] | | Cell | |
| | | [symbol] | [symbol] | [symbol] | | | | [symbol] | [symbol] | [symbol] | Separation | |
| Growth Factor | Full Name | Baseline | [symbol] | [symbol] | [symbol] | [symbol] | [symbol] | [symbol] | [symbol] | [symbol] | Method | AMP |
| aFGF | acidic fibroblast growth factor | | | | | | | | | | | 135,488 |
| bFGF | basic fibroblast growth factor | | | | | | | [symbol] | | | | 897,259 |
| EGF | apidermal growth factor | | | | | | | | | | | 15,439 |
| HGFa | hepatocyte growth factor activator | | | | | | | | | | | 2,178,020 |
| HGFb | hepatocyte growth factor b | | | | | | | | | | | 721,321 |
| IGF-1 | insulin-like growth factor 1 | | | | | | | [symbol] | | | 84,200 | 83,100 |
| PDGF-AA | | | | | | | | [symbol] | | | | |
| PDGF-AB | platelet derived growth factor AB | 15,416 | 68,217 | 74,280 | 80,180 | [symbol] | [symbol] | [symbol] | | | 117,500 | |
| PDGF-BB | platelet derived growth factor BB | | | | | | | [symbol] | | | 9,900 | 192,215 |
| TGF-β1 | transforming growth factor β1 | 14,000 | 47,302 | 44,222 | [symbol] | 7,754 | 6,472 | [symbol] | | | 108,400 | 74,058 |
| VEGF | vascular [symbol] growth factor | [symbol] | [symbol] | [symbol] | [symbol] | [symbol] | [symbol] | [symbol] | [symbol] | [symbol] | | 58,571 |
| SDF1α | stromal cell derived factor 1 | | | | | | | | [symbol] | [symbol] | | |
| PDGF (subunits | platelet derived growth factor | | | | | | | | [symbol] | [symbol] | | |
| undefined) | | | | | | | | | | | | |
| TGF-β2 | transforming growth factor β2 | | | | | | | | | | 400 | |

all values are in pg/ml [symbol]
indicates data missing or illegible when filed

### Example 30

Table 11 below demonstrates concentrations of bioactive intracellular components of embodiments of tissue implants according to the present disclosure as described herein (proteiOS and AMP) prepared by methods as described herein. The values listed demonstrate embodiments of effective amounts to deliver to subjects in need thereof according to embodiments of the present disclosure.

| | | | | [target] (pg/ml) |
|---|---|---|---|---|
| | | | | intended in AMP |
| | | [target] (ng/ml) | [target] (pg/ml) | (10x dilution of |
| Target | Full Name | in ProteiOS | in ProteiOS | ProteiOS) |
| aFGF | acidic fibroblast growth factor | 1,354.86 | 1,354,683.10 | 135,488.31 |
| bFGF | basic fibroblast growth factor | 8,972.50 | 8,972,504.04 | 697,250.40 |
| BMP-4 | bone morphogenetic protein 4 | 237.47 | 237,474.53 | 23,747.45 |
| BMP-6 | bone morphogenetic protein 6 | 219.89 | 219,893.58 | 21,989.36 |
| BMP-7 | bone morphogenetic protein 7 | 699.37 | 699,368.14 | 69,936.81 |
| BMP-9 | bone morphogenetic protein 9 | 1,438.81 | 1,438,612.89 | 143,881.29 |
| EGF | epidermal growth factor | 154.39 | 154,391.34 | 15,439.13 |
| HGFa | hepatocyte growth factor activator | 21,760.20 | 21,760,195.74 | 2,176,019.57 |
| HGFb | hepatocyte growth factor b | 7,213.21 | 7,213,206.22 | 721,320.62 |
| IGF-1 | insulin-like growth factor 1 | 631.00 | 631,000.32 | 63,100.03 |
| OPG | osteoprotegerin | 1,023.56 | 1,023,560.07 | 102,356.01 |
| OPN | osteopontin | 373.12 | 373,115.05 | 37,311.50 |
| PDGF-BB | platelet derived growth facter BB | 1,922.15 | 1,922,150.82 | 192,215.08 |
| TGF-β1 | transforming growth factor β1 | 740.56 | 740,558.10 | 74,055.81 |
| VEGF | vascular endothelial growth factor | 665.71 | 665,705.40 | 66,570.54 |

1. A method of improving hair growth or hair quality comprising:
delivering a tissue implant to a subject in need thereof by a delivery method,
wherein the tissue implant comprises cell lysate comprising a bioactive intracellular component.

2. The method of claim 1, wherein the tissue implant is derived from an autologous donor, an allogeneic donor, a xenogeneic donor, a syngeneic donor, and combinations thereof.

3. The method of claim 1, wherein the tissue implant is derived from a physiological solution comprising blood cells, bone marrow, bone marrow cells, amniotic fluid, amniotic fluid cells, amnion, amnion ECM, placenta, placental ECM, muscle, muscle ECM, interstitial fluid, stromal vascular fraction, or synovial fluid, individually or in combination.

4. The method of claim 1, wherein the cell lysate is derived from tissue containing one or more adipose cells, tissue containing one or more bone marrow cells, tissue containing one or more amnion cells, tissue containing one or more blood cells, tissue containing one or more dermal cells, or combinations thereof.

5. The method of 1, wherein the cell lysate is derived from mesenchymal stem cells.

6. The method of claim 1, wherein the cell lysate is derived from adipose derived stem cells.

7. The method of claim 1, wherein the tissue implant further comprises one or more of: a delivery enhancer, amino acid, peptide, flow enhancer, preservative, storage agent, protease inhibitor, or a stabilizer.

8. The method of claim 1, wherein the delivery method is surgical implantation, subdermal injection, topical application, microneedling, transdermal application, or combinations thereof.

9. The method of claim 1, wherein the tissue implant is terminally sterilized, cross-linked, or both using irradiation or chemical means.

10. The method of claim 1, wherein the irradiation is gamma irradiation, x-ray irradiation, uv irradiation, or ebeam irradiation.

11. The method of claim 1, wherein the tissue implant further comprises a carrier substrate.

12. The method of claim 11, wherein the carrier substrate is selected from the group consisting of: a complete extracellular matrix, a decellularized extracellular matrix, extracellular matrix components, a hydrogel, an amino acid, a polymer solid, a polymer semi-solid, a carbohydrate, self-assembling peptides, carbon nanotubes, chitosan, alginate, bone powder, cartilage powder, a protein, a sugars, a plastic, a metal, a collagen, and combinations thereof.

13. The method of claim 1, wherein the wherein the bioactive intracellular component is contained in a slurry, and wherein the slurry ratio of slurry to carrier substrate is about 100:1 (v/v) to about 1:100 (v/v).

14. The method of claim 1, wherein the bioactive intracellular component is present in the tissue implant at a concentration of at least at least 1 pg/g.

15. The method of claim 1, wherein the bioactive intracellular component is present in the tissue implant at a concentration of about 0 pg/g to about 100 mg/g.

16. The method of claim 1, wherein the bioactive intracellular component is selected from the following group consisting of: a platelet-derived growth factor, a hepatocyte growth factor, an insulin growth factor, an angiopoietin, a fibronectin, a transforming growth factor, a nerve growth factor, a fibronectin, an integrin, a bone morphogenetic protein, an epidermal growth factor, an insulin-like growth factor, a fibroblast growth factor, vascular endothelial growth factor, osteoprotegerin, and osteopontin, and combinations thereof.

17-26. (canceled)

27. The method of claim 1, further comprising adding a compound from the group consisting of: preservatives, antibiotics, antivirals, antifungals, pH stabilizers, osmostablizers, anti-inflammants, anti-neoplastics, growth factors, angiogenic compounds, vasculogenic compounds, chemotherapeutics, immunomodulators, chemoattractants, and combinations thereof to the intracellular component, the carrier substrate or the combined bioactive intracellular component-carrier substrate.

28-37. (canceled)

38. A kit, comprising a tissue implant in an amount effective to stimulate hair growth or hair repair in a subject in need thereof.

39. (canceled)

40. A method of improving hair growth or hair quality in a subject in need thereof comprising:
delivering a tissue implant to a subject in need thereof by a delivery method,
wherein the tissue implant comprises cell lysate comprising a bioactive intracellular component; and
wherein the tissue implant is delivered in an amount effective to improve hair growth or hair quality.

41-80. (canceled)
